(19) United States
(12) Patent Application Publication
(10) Pub. No.: US 2017/0239289 A1
(43) Pub. Date: Aug. 24, 2017

(54) TREATMENT OF INFLAMMATION, RESPIRATORY TRACT INFECTIONS AND CYSTIC FIBROSIS

(71) Applicant: Advanced Inhalation Therapies (AIT) Ltd., Rehovot (IL)

(72) Inventors: Yossef Av-Gay, Vancouver (CA); David Greenberg, Omer (IL); Racheli Vizman, Beer-Yaacov (IL)

(73) Assignee: Advanced Inhalation Therapies (AIT) Ltd., Rehovot (IL)

(21) Appl. No.: 15/504,566

(22) PCT Filed: Aug. 24, 2015
(86) PCT No.: PCT/IB15/01920

§ 371 Date: Feb. 16, 2017

Related U.S. Application Data
(60) Provisional application No. 62/041,272, filed on Aug. 25, 2014.
(60) Provisional application No. 62/041,258, filed on Aug. 25, 2014.

(51) Int. Cl. A61K 33/00; G01N 33/84

(52) U.S. Cl. CPC A61K 33/00; G01N 33/84; G01N 2800/52

(57) ABSTRACT

The present invention provides a method for treating a disease in a human subject in need thereof, wherein the disease is selected from the group consisting of inflammation, bronchiolitis and cystic fibrosis, and wherein the method comprises repeatedly administering to the human subject a gas mixture comprising nitric oxide at a concentration from about 144 to about 176 ppm for a first period of time, followed by a gas mixture containing no nitric oxide for a second period of time, wherein the administration is repeated for a time sufficient to: a) reduce the level of at least one inflammatory biomarker in the human subject when compared to the level of the inflammatory biomarker prior to the administration; b) reduce the microbial density by 1 to 2 log units as measured by colony forming units in the human subject when compared to the microbial density prior to the administration; or c) a combination thereof.

### CROSS REFERENCE TO RELATED APPLICATIONS

This application claims priority to U.S. Provisional Patent Application No. 62/041,258, filed Aug. 25, 2014, and U.S. Provisional Patent Application No. 62/041,272, filed Aug. 25, 2014, the contents of which are hereby incorporated by reference in their entirety.

### FIELD OF THE INVENTION

The present invention, in some embodiments thereof, relates to therapy, and more particularly, but not exclusively, to methods and devices for treating inflammation, respiratory tract infections or cystic fibrosis in human subjects.

### BACKGROUND OF THE INVENTION

Inflammation is part of the complex biological response of vascular tissues to harmful stimuli, such as pathogens, damaged cells, or irritants. The classical signs of acute inflammation are pain, heat, fever, redness, swelling, and loss of function.

Respiratory tract infections (RTIs) are a major cause of hospitalization, economic burden, mortality, and morbidity worldwide. In the United States (US) alone, RTIs result in over 1.5 million hospitalizations annually. Bronchiolitis is defined as an infection of the small airways. It is also the most common manifestation of acute lower respiratory infection (ALRI) in early infancy, and is the leading cause of global child mortality. Viral bronchiolitis is currently the most common reason for pediatric hospital admission in the US, accounting for almost 20% of all-cause infant hospitalizations.

Cystic fibrosis (CF) is an inherited monogenic disorder that presents as a multisystem disease that causes severe lung damage and nutritional deficiencies. CF affects cells that produce mucus, sweat, and digestive juices. The defective gene causes these secretions to become thick and sticky and affect the ability of organs such as the lungs and pancreas to function efficiently. Human subjects diagnosed with, or suffering from CF are highly prone to environmental opportunistic bacterial infections leading to prolonged and chronic lung infections. This results in reduction in the life expectancy of human subjects diagnosed with or suffering from CF due to excessive lung tissue destruction.

### SUMMARY OF THE INVENTION

According to some embodiments of the present invention, there is provided a method of treating an inflammatory disease, a respiratory tract infection, or cystic fibrosis or disorder in a human subject, which comprises subjecting the human subject to intermittent inhalation of a gas mixture comprising nitric oxide at a concentration of at least 160 ppm, thereby treating the inflammatory disease, respiratory tract infection, or cystic fibrosis.

According to some of the embodiments presented herein, a level of at least one of inflammatory biomarker in the human subject is reduced.

According to some embodiments of the present invention, there is provided a method of reducing a level of an inflammatory biomarker in a human subject, which comprises subjecting the human subject to intermittent inhalation of a gas mixture comprising nitric oxide at a concentration of at least 160 ppm, thereby reducing the level of the inflammatory biomarker.

According to some of the embodiments presented herein, the inflammatory biomarker is selected from the group consisting of C-reactive protein (CRP), TNFα, TNF RII, IL-1β, IL-1ra/IL-1F3, IL-2, IL-4, IL-5, IL-6, IL-8, CXCL8/IL-8, IL-10, IL-12 p70, IL-17A, GM-CSF, ICAM-1, IFN-gamma, MMP-8, MMP-9, VEGF and IL-12p70, neutrophils, lymphocytes and eosinophils count, neutrophil elastase activity, alpha-1-antitrypsin (AAT), haptoglobin, transferrin, an immunoglobulin, granzyme B (GzmB), eosinophil cationic protein (ECP), eotaxin, tryptase, chemokine C-C motif ligand 18 (CCL18/PARC), RANTES (CCL5), surfactant protein D (SP-D), lipopolysaccharide (LPS)-binding protein and soluble cluster of differentiation 14 (sCD14).

According to some of the embodiments presented herein, the inflammatory biomarker is C-reactive protein (CRP).

According to some of the embodiments presented herein, the inflammatory biomarker is selected from the group consisting of neutrophils count, IL-8 and neutrophil elastase activity.

According to some embodiments of the present invention, there is provided a method of reducing a level of C-reactive protein (CRP) in a human subject in need thereof, which comprises subjecting the human subject to intermittent inhalation of a gas mixture comprising nitric oxide at a concentration of at least 160 ppm, thereby reducing the level of CRP.

According to some of the embodiments presented herein, the level of any of the biomarkers described herein is reduced by at least 5 percent.

According to some embodiments, the human subject suffers from a microbial infection associated with cystic fibrosis.

According to some embodiments, the microbial infection is caused by a pathogenic microorganism.

According to some embodiments, the pathogenic microorganism is selected from the group consisting of P. alcaligenes, non-mucoid and mucoid Pseudomonas aeruginosa, A. fumigates, Staphylococcus aureus, Haemophilus influenza, Burkholderia cepacia complex, Klebsiella pneumonia, Escherichia coli, methicillin-resistant Staphylococcus aureus (MRSA), methicillin-sensitive Staphylococcus aureus (MSSA), Stenotrophomonas maltophilia, Achromobacter spp., Achromobacter xylosoxidans and non-tuberculous mycobacteria (NTM) species.

According to some embodiments, the pathogenic microorganism is selected from the group consisting of P. alcaligenes, methicillin-sensitive Staphylococcus aureus (MSSA), Achromobacter spp., A. fumigates, non-mucoid P. aeruginosa and mucoid P. aeruginosa.

According to some embodiments, the load of the pathogenic microorganism is reduced by at least 1 log units during the intermittent inhalation.

According to some of the embodiments presented herein, the level of at least one inflammatory biomarker associated with cystic fibrosis in the subject is reduced during the intermittent inhalation.

According to some embodiments, the inflammatory biomarker associated with cystic fibrosis is selected from the group consisting of C-reactive protein (CRP), a cytokine, alpha-1-antitrypsin (AAT), haptoglobin, transferrin, an immunoglobulin, granzyme B (GzmB), chemokine C-C motif ligand 18 (CCL18/PARC), surfactant protein D (SP-D), lipopolysaccharide (LPS)-binding protein and soluble cluster of differentiation 14 (sCD14).

According to some embodiments, the inflammatory biomarker associated with cystic fibrosis is C-reactive protein (CRP).

According to some embodiments, the level of the CRP is reduced by at least 10 percent during the intermittent inhalation.

According to some embodiments, the cytokine is selected from the group consisting of TNFα, IL-1β, IL-6, IL-8, IL-10 and IL-12p70.

According to some embodiments, the cytokine is selected from the group consisting of IL-6 and IL-1.

According to some embodiments, the level of the cytokine is reduced by at least 5 percent during the intermittent inhalation.

According to some embodiments of the present invention there is provided a method of reducing a load of a pathogenic microorganism in a human subject in need thereof, the method comprising subjecting the human subject to intermittent inhalation of a gas mixture comprising nitric oxide at a concentration of at least 160 ppm, thereby reducing the load of the pathogenic microorganism in the human subject, wherein the pathogenic microorganism is selected from the group consisting of P. alcaligenes, non-mucoid and mucoid Pseudomonas aeruginosa, A. fumigates, Staphylococcus aureus, Haemophilus influenza, Burkholderia cepacia complex, Klebsiella pneumonia, Escherichia coli, methicillin-resistant Staphylococcus aureus (MRSA), methicillin-sensitive Staphylococcus aureus (MSSA), Stenotrophomonas maltophilia, Achromobacter spp., Achromobacter xylosoxidans and non-tuberculous mycobacteria (NTM) species.

According to some embodiments, the pathogenic microorganism is selected from the group consisting of P. alcaligenes, methicillin-sensitive Staphylococcus aureus (MSSA), Achromobacter spp., A. fumigates, non-mucoid P. aeruginosa and mucoid P. aeruginosa.

According to any one of the embodiments of the present invention, the load of the pathogenic microorganism is reduced by at least 1 log units during the intermittent inhalation.

According to any one of the embodiments of the present invention, the human subject is afflicted by cystic fibrosis.

According to some embodiments of the present invention there is provided a method of reducing a level of an inflammatory biomarker associated with cystic fibrosis in a human subject having or afflicted by cystic fibrosis, the method comprising subjecting the human subject to intermittent inhalation of a gas mixture comprising nitric oxide at a concentration of at least 160 ppm, thereby reducing the level of an inflammatory biomarker.

According to some embodiments, the inflammatory biomarker associated with cystic fibrosis is C-reactive protein (CRP).

According to some embodiments, the level of the CRP is reduced by at least 10 percent during the treatment.

According to some embodiments, the inflammatory biomarker associated with cystic fibrosis is selected from the group consisting of IL-6 and IL-1β.

According to some embodiments, the level of the inflammatory biomarker associated with cystic fibrosis is reduced by at least 5 percent.

According to some of the embodiments presented herein, the method further comprises monitoring at least one on-site oximetric parameter in the subject selected from the group consisting of:

perfusion index (PI);

respiration rate (RRa);

oxyhemoglobin saturation (SpO₂);

total hemoglobin (SpHb);

carboxyhemoglobin (SpCO);

methemoglobin (SpMet);

oxygen content (SpOC); and

pleth variability index (PVI).

According to some of the embodiments presented herein, the method further comprises monitoring at least one on-site spirometric parameter in the subject selected from the group consisting of:

forced expiratory volume (FEV₁);

maximum mid-expiratory flow (MMEF)

diffusing capacity of the lung for carbon monoxide (D_{L}CO);

forced vital capacity (FVC);

total lung capacity (TLC); and

residual volume (RV).

According to some of the embodiments presented herein, the method further comprises monitoring at least one on-site parameter in the gas mixture inhaled by the subject, selected from the group consisting of:

end tidal CO₂ (ETCO₂);

nitrogen dioxide (NO₂),

nitric oxide (NO); and

fraction of inspired oxygen (FiO₂).

According to any one of the embodiments of the present invention, the method further comprising monitoring at least one on-site parameter in the subject, the at least one on-site parameter being selected from the group consisting of:

Oxyhemoglobin Saturation (SpO₂); and

Methemoglobin (SpMet).

According to some embodiments, the at least one parameter comprises SpMet and during and following the intermittent inhalation, the SpMet is increased by less than 5%.

According to some embodiments, the at least one parameter comprises SpO₂ and during the intermittent inhalation, a level of the SpO₂ is higher than 89%.

According to some of the embodiments presented herein, the method further comprises monitoring at least one off-site bodily fluid parameter in the subject selected from the group consisting of serum nitrite/nitrate (NO₂⁻/NO₃⁻) and urine nitrite/nitrate.

According to some embodiments, the at least one parameter comprises serum nitrite/nitrate level and during and following the intermittent inhalation, a level of the serum nitrite is less than 2.5/25 micromole per liter respectively.

According to some of the embodiments presented herein, the method further comprises monitoring at least one off-site bodily fluid parameter in the subject selected from the group consisting of:

a bacterial and/or fungal load;

blood methemoglobin;

blood pH;

a coagulation factor;

blood hemoglobin;

hematocrit ratio;

red blood cell count;

white blood cell count;

platelet count;

vascular endothelial activation factor;

renal function;

an electrolyte;

a complete blood count;

a pregnancy hormone;

serum creatinine; and liver function.

According to some of the embodiments presented herein, the intermittent inhalation comprises at least one cycle of continuous inhalation of the mixture for a first time period, followed by inhalation of no nitric oxide for a second time period.

According to some embodiments, the first time period is about 30 minutes.

According to some embodiments, the second time period ranges from 3 to 5 hours.

According to some embodiments, the intermittent inhalation comprises from 1 to 6 of the cycles per day.

According to some embodiments, the intermittent inhalation comprises 5 of the cycles per day.

According to some embodiments, the intermittent inhalation is effected over a time period that ranges from 1 day to 3 weeks.

According to some embodiments, during the first time period, the concentration of nitric oxide in the mixture deviates from the concentration of at least 160 ppm by less than 10%.

According to some embodiments, during the first time period, a concentration of NO₂ in the mixture is less than 5 ppm.

According to some embodiments, during the first time period, a concentration of O₂ in the mixture ranges from 20% to 25%.

According to some embodiments, during the first time period, a fraction of inspired oxygen level (FiO₂) in the mixture ranges from 21% to 100%.

According to some embodiments, at least one of the monitored parameters is SpMet and during and following the subjecting, the SpMet is increased by less than 5%.

According to some embodiments, at least one of the monitored parameters is SpO₂ and during the subjecting, a level of the SpO₂ is higher than 89%.

According to some embodiments, at least one of the monitored parameters is serum nitrite/nitrate level and during and following the subjecting, a level of the serum nitrite is less than 2.5/25 micromole per liter respectively.

According to some of the embodiments presented herein, the disease or disorder is selected from the group consisting of an idiopathic inflammatory disease or disorder, a chronic inflammatory disease or disorder, an acute inflammatory disease or disorder, an autoimmune disease or disorder, an infectious disease or disorder, an inflammatory malignant disease or disorder, an inflammatory transplantation-related disease or disorder, an inflammatory degenerative disease or disorder, a disease or disorder associated with a hypersensitivity, an inflammatory cardiovascular disease or disorder, an inflammatory cerebrovascular disease or disorder, a peripheral vascular disease or disorder, an inflammatory glandular disease or disorder, an inflammatory gastrointestinal disease or disorder, an inflammatory cutaneous disease or disorder, an inflammatory hepatic disease or disorder, an inflammatory neurological disease or disorder, an inflammatory musculo-skeletal disease or disorder, an inflammatory renal disease or disorder, an inflammatory reproductive disease or disorder, an inflammatory systemic disease or disorder, an inflammatory connective tissue disease or disorder, an inflammatory tumor, necrosis, an inflammatory implant-related disease or disorder, an inflammatory aging process, an immunodeficiency disease or disorder, a proliferative disease or disorder and an inflammatory pulmonary disease or disorder.

According to further aspects of the present invention, there are provided devices and systems for effecting the intermittent inhalation described herein in any of the methods described herein, as is detailed hereinafter.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

FIGS. 1A-B present comparative bar plots, showing average change in MetHb percent levels (FIG. 1A) and NO₂ levels in ppm (FIG. 1B) prior to first treatment (blue) and after last treatment (red) (threshold value of 5% is shown as a dotted red line), as measured in 9 human subjects during 10 days of treatment, according to some embodiments of the present invention.

FIG. 2 A-F present results of CFU determination of P. alcaligenes in “Patient 1” (CFSCH01) (FIG. 2A), MSSA in “Patient 3” (CFSCH03) (FIG. 2B), Achromobacter spp. in “Patient 3” (FIG. 2C), A. fumigatus in “Patient 3” (FIG. 2D), non-mucoid P. aeruginosa in “Patient 4” (CFSCH04) (FIG. 2E), and mucoid P. aeruginosa in “Patient 4” (FIG. 2F), throughout the treatment, whereas “nd” stands for non-detected levels.

FIG. 3 presents a comparative plot showing the linear trend of FEV₁ measurements as taken from 9 human subjects diagnosed with CF treated with 160 ppm nitric oxide three times/day with at least 3.5 hours between treatments for 10 days from screening to end of treatment, according to some embodiments of the present invention.

FIG. 4 presents a comparative plot showing the linear trend of CRP levels in mg/L as measured in 9 human subjects diagnosed with CF treated with 160 ppm nitric oxide three times/day with at least 3.5 hours between treatments for 10 days from screening to end of treatment, according to some embodiments of the present invention.

FIG. 5 presents an outline of a method for treating bronchiolitis according to an embodiment of the present invention.

FIG. 6 presents the percentage of subjects with a MetHb greater than or less than 5% during treatment with nitric oxide according to one embodiment of the present invention.

FIG. 7 presents the mean MetHb levels over time for treatment 1 in subjects treated with nitric oxide according to one embodiment of the present invention.

FIG. 8 presents the mean MetHb levels over time according to treatment number in subjects treated with nitric oxide according to one embodiment of the present invention.

FIG. 9 presents median length of stay (LOS) in human subjects diagnosed with acute bronchiolitis treated according to the methods of one embodiment of the present invention. Panel A shows ITT. Panel B shows the subgroup of subjects with a LOS less than or equal to 24 hours. Panel C shows the subgroup of subjects with a LOS greater than 24 hours.

FIG. 10 presents the Kaplan-Meier Analysis of data from human subjects diagnosed with acute bronchiolitis treated according to the methods of one embodiment of the present invention.

FIG. 11 presents median length of stay (LOS) in human subjects diagnosed with acute bronchiolitis treated according to the methods of one embodiment of the present invention. Panel A shows PP. Panel B shows the subgroup of subjects with a LOS less than or equal to 24 hours. Panel C shows the subgroup of subjects with a LOS greater than 24 hours.

FIG. 12 presents median LOS according to treatment group for subjects with LOS greater than 24 Hours, LOS greater than 36 Hours, and 10 most severe (mITT).

FIG. 13 presents median time to first O₂ saturation sustained to discharge, according to treatment subgroup (ITT) in human subjects diagnosed with acute bronchiolitis treated according to the methods of one embodiment of the present invention. Panel A shows ITT. Panel B shows the subgroup of subjects with a LOS less than or equal to 24 hours. Panel C shows the subgroup of subjects with a LOS greater than 24 hours.

FIG. 14 presents the Kaplan-Meier Analysis of data from human subjects diagnosed with acute bronchiolitis treated according to the methods of one embodiment of the present invention.

FIG. 15 presents median time to first O₂ saturation sustained to discharge, according to treatment subgroup (PP) in human subjects diagnosed with acute bronchiolitis treated according to the methods of one embodiment of the present invention. Panel A shows PP. Panel B shows the subgroup of subjects with a LOS less than or equal to 24 hours. Panel C shows the subgroup of subjects with a LOS greater than 24 hours.

FIG. 16 presents median time to first O₂ saturation sustained to discharge according to treatment group for subjects with LOS greater than 24 Hours, LOS greater than 36 Hours, and 10 most severe (mITT).

FIG. 17 presents median time to clinical score less than or equal to 5, according to treatment subgroup (ITT) in human subjects diagnosed with acute bronchiolitis treated according to the methods of one embodiment of the present invention. Panel A shows ITT. Panel B shows the subgroup of subjects with a LOS less than or equal to 24 hours. Panel C shows the subgroup of subjects with a LOS greater than 24 hours.

FIG. 18 presents the Kaplan-Meier Analysis of data from human subjects diagnosed with acute bronchiolitis treated according to the methods of one embodiment of the present invention.

FIG. 19 presents median time to clinical score less than or equal to 5, according to treatment subgroup (PP) in human subjects diagnosed with acute bronchiolitis treated according to the methods of one embodiment of the present invention. Panel A shows PP. Panel B shows the subgroup of subjects with a LOS less than or equal to 24 hours. Panel C shows the subgroup of subjects with a LOS greater than 24 hours.

FIG. 20 presents median time to clinical score less than or equal to 5 according to treatment group for subjects with LOS greater than 24 Hours, LOS greater than 36 Hours, and 10 most severe (mITT).

FIG. 21 presents an outline of a method for treating cystic fibrosis according to an embodiment of the present invention.

FIG. 22 presents mean pre and post-treatment MetHb levels by treatment number for subjects suffering from cystic fibrosis treated according to an embodiment of the present invention.

FIG. 23 presents mean pre and post-treatment FEV₁ for subjects suffering from cystic fibrosis treated according to an embodiment of the present invention.

FIG. 24 presents bacterial and fungal load in subjects suffering from cystic fibrosis treated according to an embodiment of the present invention.

FIG. 25 presents CRP levels in subjects suffering from cystic fibrosis treated according to an embodiment of the present invention.

### DESCRIPTION OF SPECIFIC EMBODIMENTS OF THE INVENTION

The present invention, in some embodiments thereof, relates to therapy, and more particularly, but not exclusively, to methods and devices for treating inflammation, respiratory tract infections or cystic fibrosis in human subjects.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Inflammation is a primary or secondary response of the body to cell damage, infection or the presence of foreign matter. As a primary factor, inflammation is associated with a large number of diseases and disorders that may also cause system deterioration and failure and be the cause of secondary conditions, if goes untreated. Apart of the typical symptoms of inflammation, such as fever, swelling, pain and the likes, inflammation is also diagnosed by monitoring certain endogenous factors or inflammatory biomarkers, the level of which in the body is indicative of the severity and the stage of the inflammation.

Cystic fibrosis (CF) is a genetic disorder in which mutations in the epithelial chloride channel, CF transmembrane conductance regulator (CFTR), impairs various mechanism of innate immunity. Chronic lung infections caused by pathogenic microorganisms are the leading cause of morbidity and mortality in human subjects diagnosed with, or suffering from CF. Early antibiotic eradication treatment of human subjects diagnosed with, or suffering from CF for the most prevalent bacterial pathogen, Pseudomonas aeruginosa, has considerably increased the life expectancy in CF, however still the vast majority of adult human subjects diagnosed with, or suffering from CF suffer from chronic lung infections which are difficult to treat due to biofilm formation and the development of antibiotic resistant strains of the virulent. Other species found in CF airways include antibiotic resistant strains such as methicillin-resistant S. aureus (MRSA), members of the Burkholderia cepacia complex, Haemophilus influenzae, Stenotrophomonas maltophilia, Achromobacter xylosoxidans, non-tuberculous mycobacteria (NTM) species and various strict anaerobic bacteria. In addition, poor clearance of mucus from the bronchi causes general breathing difficulties in human subjects diagnosed with, or suffering from CF.

Bronchiolitis is defined as an infection of the small airways. It is also the most common manifestation of acute lower respiratory infection (ALRI) in early infancy, and is the leading cause of global child mortality. Viral bronchiolitis is currently the most common reason for pediatric hospital admission in the US, accounting for almost 20% of all-cause infant hospitalizations. Viral etiology is the main cause, and among the respiratory viruses, respiratory syncytial virus (RSV) is believed to be the most important viral pathogen causing ALRI in young children. The disease is common mainly in the first year of life. The clinical signs and symptoms are consistent with hypoxia, difficulty breathing, coryza, poor feeding, cough, wheeze and crepitations on auscultation, and in some cases respiratory failure.

In some aspects of the present invention, intermittent dosing and delivery by inhalation of nitric oxide, cycling between high concentrations of nitric oxide for a relatively short period of time and longer periods of no or low concentration of nitric oxide has been shown to overcome the problems of nitric oxide toxicity in humans of all ages. In some embodiments, it has been shown that the high concentration of nitric oxide, delivered according to an intermittent regimen, is effective in overwhelming the nitric oxide defense mechanisms of pathogens, and hence that at such a high concentration, nitric oxide exhibits a pronounced anti-microbial effect.

In one embodiment, the present invention provides a method that administers nitric oxide to a human subject, wherein the administration is short durations of high concentrations of nitric oxide, that reduces the level of exemplary inflammatory biomarkers, while not causing lung injury or other signs of adverse effects. For example, the present inventors have surprisingly uncovered that C-reactive protein (CRP) levels were improved (reduced) as a result of subjecting a human patient to a treatment of intermittent inhalation of nitric oxide at a concentration of at least 160 ppm. Levels of at least some inflammatory cytokines are also reduced as a result of subjecting a human patient to a treatment of intermittent inhalation of nitric oxide at a concentration of at least 160 ppm. These data indicates that intermittent inhalation of nitric oxide as described herein can be beneficially used in treating inflammation and disease and disorders associated with inflammation (inflammatory diseases and disorders).

In one embodiment, it has also been found that nitric oxide plays a part in the movement of cilia in the lungs, the inflammatory pathway, and the immune system—all processes that, if augmented, aid in the treatment of CF.

In one embodiment, the present invention provides a method that administers nitric oxide to a human subject, wherein the administration is short durations of high concentrations of nitric oxide, that improves lung function and reduce microbial infections and inflammatory symptoms in human subjects diagnosed with, or suffering from CF, while not causing lung injury or other signs of adverse effects. In one embodiment, forced expiratory volume in 1 sec (FEV) and C-reactive protein (CRP) levels were improved and serum nitrites/nitrates did not differ between baseline and the study period, while methemoglobin levels increased period up to a tolerated and accepted levels. It was thus demonstrated that intermittent inhalation of 160 ppm nitric oxide or more is safe and well tolerated in human subjects diagnosed with, or suffering from CF and is beneficial in terms of alleviation of CF symptoms.

In one embodiment, the present invention administers nitric oxide to a human subject, wherein the administration is short durations of high concentrations of nitric oxide, that improves lung function in human subjects suffering from bronchiolitis, while not causing lung injury or other signs of adverse effects.

Herein-throughout, whenever the term “nitric oxide” is used in the context of inhalation, it is to be understood that nitric oxide is inhaled in the gaseous state.

Treatment of Inflammation and of Diseases and Disorders Associated Therewith:

According to some embodiments of the present invention, there is provided a method of treating inflammation and/or an inflammatory disease or disorder in a human subject, which is effected by subjecting the human subject to a treatment of intermittent inhalation of a gas mixture comprising nitric oxide at a concentration of at least 160 ppm.

According to embodiments of the present invention, the method of treating inflammatory disease or disorder encompasses any beneficial effect which is exhibited in a patient in need thereof, including amelioration of a symptom of inflammation, amelioration of an adverse effect caused by a medical condition associated with inflammation, amelioration of an adverse effect caused by another treatment of inflammation and of its symptoms, reduction of mortality in inflammation human subjects and general improvement of the medical and mental condition of a human subject.

As discussed hereinabove, some of the signs of an inflammatory condition include a change, typically an increase, in the detected levels of some proteins, referred to herein as inflammatory biomarkers, which play key roles in human immune response. Thus, reduction in inflammatory biomarkers is typically regarded as a beneficial effect of a treatment; and consequently, a reduction in a level of an inflammatory biomarker can be used as an indication of treatment of inflammation as described herein.

It should be noted that according to some embodiments of the present invention, the term “inflammatory biomarker” is used in the context of an indication of inflammation and a mean to monitor the progress of a treatment. In some embodiments, any of the methods described herein is effected while monitoring various physiological parameters and various biomarkers, such as inflammatory biomarkers, in the subject, in order to follow the progression of the disease and/or the progression of the treatment.

It should also be noted that according to some embodiments of the present invention, the term “inflammatory biomarker” is used in the context of the treatment by itself, namely the reduction of a level of an inflammatory biomarker, which is involved in inflammatory processes, results in inhibiting, and therefore treating a disease or disorder associated with inflammation.

According to some embodiments of the present invention, there is provided a method of reducing a level of an inflammatory biomarker in a human subject in need thereof, which is effected by subjecting the human subject to a treatment of intermittent inhalation of a gas mixture comprising nitric oxide at a concentration of at least 160 ppm.

Inflammatory biomarkers, which may be targeted for reduction by the presently claimed method according to some embodiments thereof, include without limitation, C-reactive protein (CRP), TNFα, TNF RII, IL-1β, IL-1ra/IL-1F3, IL-2, IL-4, IL-5, IL-6, IL-8, CXCL8/IL-8, IL-10, IL-12 p70, IL-17A, GM-CSF, ICAM-1, IFN-gamma, MMP-8, MMP-9, VEGF and IL-12p70, neutrophils, lymphocytes and eosinophils count, neutrophil elastase activity, alpha-1-antitrypsin (AAT), haptoglobin, transferrin, an immunoglobulin, granzyme B (GzmB), eosinophil cationic protein (ECP), eotaxin, tryptase, chemokine C-C motif ligand 18 (CCL18/PARC), RANTES (CCL5), surfactant protein D (SP-D), lipopolysaccharide (LPS)-binding protein and soluble cluster of differentiation 14 (sCD14).

The term “cytokine”, as used in the context of embodiments of the present invention, encompasses chemokines, interferons, interleukins, lymphokines and tumor necrosis factor.

Following is a brief description of non-limiting exemplary inflammatory biomarkers.

Tumor necrosis factors, or the TNF family, is a group of cytokines that can cause cell death (apoptosis). The most common TNF include, without limitation, tumor necrosis factor (TNF), formerly known as TNFα or TNF alpha, and lymphotoxin-alpha, formerly known as tumor necrosis factor-beta (TNF-β). For example, TNFα is known as a cytokine, or a cell-signaling protein that signals to the body to bring the neutrophil white blood cells to the site of infection or injury. TNFα acts like a “first responder” at an accident by signaling to the body where the most damage is so that the immune system can respond effectively, which is to send neutrophils.

Nuclear factors (NF) constitute a family of closely related transcription factors which constitutively bind as dimers to specific sequences of DNA with high affinity. Family members contain an unusual DNA binding domain that binds to the recognition sequence. For example, Nuclear Factor kappa B (NFkB) is a transcription factor protein complex that acts as a switch for certain genes. When NFkB is allowed to enter the nucleus, which it does through the aid of TNFα, it turns on the genes which allow cells to proliferate, mature, and avoid destruction through apoptosis (programmed cell death). This allows white blood cells to replicate and effect their activity in cleaning up the infected or injured area. NFkB is similar to the priority setting on a communications line by opening all channels available for the quickest response.

Interleukins constitute a group of secreted proteins and signaling molecules (cytokines) that are expressed by white blood cells (leukocytes). The function of the immune system depends in a large part on interleukins, and rare deficiencies of a number of them have been described, all featuring inflammatory conditions, autoimmune diseases or immune deficiencies. The majority of interleukins are synthesized by helper CD4 T lymphocytes, as well as through monocytes, macrophages, and endothelial cells. Interleukins promote the development and differentiation of T and B lymphocytes, and hematopoietic cells.

Interleukins are typically considered in families denoted by a number, namely IL-1, IL2, IL-3, IL-4, IL-5, IL-6, IL-7, IL-8, IL-9, IL-10, IL-11, IL-12, IL-13, IL-14, IL-15, IL-16, IL-17 and so on, going from 1 to 17. An exemplary interleukin is Interleukin-6. Interleukin-6 (IL-6) is a cytokine that dictates the neutrophils to destroy themselves and draws monocytes, another type of white blood cell, to the infected or injured area instead. The monocytes create macrophages which clean up the debris and pathogens through phagocytosis, the process by which macrophages degrade dead cells and other particles whole. Another exemplary interleukin is Interleukin 8 (IL-8) or CXCL8, which is a chemokine, produced by macrophages and other cell types such as epithelial cells, airway smooth muscle cells and endothelial cells. Endothelial cells store IL-8 in their storage vesicles, the Weibel-Palade bodies. In humans, the interleukin-8 protein is encoded by the IL8 gene.

The CC chemokine (or 3-chemokine) protein family has at least 27 distinct members of this subgroup reported for mammals, called CC chemokine ligands (CCL)-1 to -28; CCL10 is the same as CCL9. Chemokines of this subfamily usually contain four cysteines (C4-CC chemokines), but a small number of CC chemokines possess six cysteines (C6-CC chemokines). C6-CC chemokines include eotaxin, CCL1, CCL15, CCL21, CCL23 and CCL28. CC chemokines induce the migration of monocytes and other cell types such as NK cells and dendritic cells. Examples of CC chemokine include monocyte chemoattractant protein-1 (MCP-1 or CCL2) which induces monocytes to leave the bloodstream and enter the surrounding tissue to become tissue macrophages. CCL5 (or RANTES) attracts cells such as T cells, eosinophils and basophils that express the receptor CCR5. Increased CCL11 levels in blood plasma are associated with aging (and reduced neurogenesis) in mice and humans.

Vascular endothelial growth factors (VEGF) is a family of signal proteins produced by cells that stimulates vasculogenesis and angiogenesis. VEGF are part of the system that restores the oxygen supply to tissues when blood circulation is inadequate. VEGF's normal function is to create new blood vessels during embryonic development, new blood vessels after injury, muscle following exercise, and new vessels (collateral circulation) to bypass blocked vessels. Serum levels of VEGF are indicative of a number of medical conditions, including inflammatory diseases and disorders. High plasma levels of VEGF are also known in bronchial asthma and diabetes mellitus.

C-Reactive Protein (CRP) is a “pattern recognition receptor” protein that is produced by the liver in response to IL-6 levels and binds to the surface of dead and dying cells, and also to certain forms of bacteria. CRP acts as a form of signal for the macrophages to ingest something through phagocytosis, and thus helps in the ultimate clearing of debris during inflammation.

According to some embodiments, monitoring the level of an inflammatory biomarker is useful in determining the course of the treatment, and therefore is a part of the method presented herein. An indication based on the monitoring of an inflammatory biomarker may cause the practitioner to change the regimen of the treatment (increase or decrease exposure of the subject to nitric oxide). In some embodiments, the level of a biomarker in, for example, a blood, serum, sputum, mucus, urine or feces extracted from the subject, based on a baseline of the serum level in the subject before commencement of the treatment, is reduced by at least 10, 15, 20, 30, 35, 40, 50 or at least 60 percent as a result of the treatment.

In some embodiments, the reduction in the level of a biomarker is not only a mean to follow the progress of the treatment, but also a goal of the treatment per-se. For example, the reduction of CRP levels is a treatment goal by itself in some general medical conditions and in inflammatory diseases.

In some embodiments, the plasma level of CRP is indicative of the progression of the inflammatory disease or disorder. In some embodiments, lowering plasma level of CRP constitutes a part of the treatment per-se.

In some embodiments of this aspect of the present invention, the inflammatory biomarker is C-reactive protein (CRP). A rate of reduction as a result of the treatment is at least 10, 15, 20, 30, 35, 40, 50 or at least 60 percent, compared to a baseline level of CRP in the patient.

Neutrophils are a type of phagocyte and are normally found in the bloodstream. During the acute phase (beginning) of inflammation, particularly as a result of bacterial infection, environmental exposure and some types of cancer, neutrophils are one of the first-responders of inflammatory cells to migrate towards the site of inflammation. Neutrophils migrate through the blood vessels, then through interstitial tissue, following chemical signals such as Interleukin-8 (IL-8), C5a, fMLP and Leukotriene B4 in a process called chemotaxis.

In some embodiments of this aspect of the present invention, the inflammatory biomarker is a level of induced sputum of neutrophils lymphocytes and eosinophils count, IL-8, eosinophil cationic protein (ECP), eotaxin, tryptase, RANTES (a C-C motif chemokine) and neutrophil elastase activity. A rate of reduction in the level of a cytokine as a result of the treatment is at least 3, 5, 10, 15, 20, 30, 35, 40, 50 or at least 60 percent, compared to a baseline level of the biomarker in the patient.

In some embodiments of this aspect of the present invention, the inflammatory biomarker is a level of blood plasma cytokine, selected from the group consisting of TNFα, TNF RII, IL-1β, IL-1ra/IL-1F3, IL-2, IL-4, IL-5, IL-6, IL-8, CXCL8/IL-8, IL-10, IL-12 p70, IL-17A, GM-CSF, ICAM-1, IFN-gamma, MMP-8, MMP-9, VEGF and IL-12p70. In some embodiments, the inflammatory biomarkers are IL-8, IL-6 and IL-10. A rate of reduction in the level of a cytokine as a result of the treatment is at least 3, 5, 10, 15, 20, 30, 35, 40, 50 or at least 60 percent, compared to a baseline level of the biomarker in the patient.

Inflammatory Diseases and Disorders:

According to some embodiments of the present invention, any of the methods based on intermittent inhalation of nitric oxide provided herein, are effective in treating, as defined herein, an inflammatory disease or disorder in a human subject.

In the context of any of the embodiments of the present invention, the inflammatory disease or disorder, or diseases or disorders associated with inflammation, include, for example and without limitation, idiopathic inflammatory diseases or disorders, chronic inflammatory diseases or disorders, acute inflammatory diseases or disorders, autoimmune diseases or disorders, infectious diseases or disorders, inflammatory malignant diseases or disorders, inflammatory transplantation-related diseases or disorders, inflammatory degenerative diseases or disorders, diseases or disorders associated with a hypersensitivity, inflammatory cardiovascular diseases or disorders, inflammatory cerebrovascular diseases or disorders, peripheral vascular diseases or disorders, inflammatory glandular diseases or disorders, inflammatory gastrointestinal diseases or disorders, inflammatory cutaneous diseases or disorders, inflammatory hepatic diseases or disorders, inflammatory neurological diseases or disorders, inflammatory musculo-skeletal diseases or disorders, inflammatory renal diseases or disorders, inflammatory reproductive diseases or disorders, inflammatory systemic diseases or disorders, inflammatory connective tissue diseases or disorders, inflammatory tumors, necrosis, inflammatory implant-related diseases or disorders, inflammatory aging processes, immunodeficiency diseases or disorders, proliferative diseases and disorders and inflammatory pulmonary diseases or disorders, as is detailed herein below.

Non-limiting examples of hypersensitivities include Type I hypersensitivity, Type II hypersensitivity, Type III hypersensitivity, Type IV hypersensitivity, immediate hypersensitivity, antibody mediated hypersensitivity, immune complex mediated hypersensitivity, T lymphocyte mediated hypersensitivity, delayed type hypersensitivity, helper T lymphocyte mediated hypersensitivity, cytotoxic T lymphocyte mediated hypersensitivity, TH1 lymphocyte mediated hypersensitivity, and TH2 lymphocyte mediated hypersensitivity.

Non-limiting examples of inflammatory cardiovascular disease or disorder include occlusive diseases or disorders, atherosclerosis, a cardiac valvular disease, stenosis, restenosis, in-stent-stenosis, myocardial infarction, coronary arterial disease, acute coronary syndromes, congestive heart failure, angina pectoris, myocardial ischemia, thrombosis, Wegener's granulomatosis, Takayasu's arteritis, Kawasaki syndrome, anti-factor VIII autoimmune disease or disorder, necrotizing small vessel vasculitis, microscopic polyangiitis, Churg and Strauss syndrome, pauci-immune focal necrotizing glomerulonephritis, crescentic glomerulonephritis, antiphospholipid syndrome, antibody induced heart failure, thrombocytopenic purpura, autoimmune hemolytic anemia, cardiac autoimmunity, Chagas' disease or disorder, and anti-helper T lymphocyte autoimmunity.

Stenosis is an occlusive disease of the vasculature, commonly caused by atheromatous plaque and enhanced platelet activity, most critically affecting the coronary vasculature.

Restenosis is the progressive re-occlusion often following reduction of occlusions in stenotic vasculature. In cases where patency of the vasculature requires the mechanical support of a stent, in-stent-stenosis may occur, re-occluding the treated vessel.

Non-limiting examples of cerebrovascular diseases or disorders include stroke, cerebrovascular inflammation, cerebral hemorrhage and vertebral arterial insufficiency.

Non-limiting examples of peripheral vascular diseases or disorders include gangrene, diabetic vasculopathy, ischemic bowel disease, thrombosis, diabetic retinopathy and diabetic nephropathy.

Non-limiting examples of autoimmune diseases or disorders include all of the diseases caused by an immune response such as an autoantibody or cell-mediated immunity to an autoantigen and the like. Representative examples are chronic rheumatoid arthritis, juvenile rheumatoid arthritis, systemic lupus erythematosus, scleroderma, mixed connective tissue disease, polyarteritis nodosa, polymyositis/dermatomyositis, Sjogren's syndrome, Bechet's disease, multiple sclerosis, autoimmune diabetes, Hashimoto's disease, psoriasis, primary myxedema, pernicious anemia, myasthenia gravis, chronic active hepatitis, autoimmune hemolytic anemia, idiopathic thrombocytopenic purpura, uveitis, vasculitides and heparin induced thrombocytopenia.

Non-limiting examples of inflammatory glandular diseases or disorders include pancreatic diseases or disorders, Type I diabetes, thyroid diseases or disorders, Graves' disease, thyroiditis, spontaneous autoimmune thyroiditis, Hashimoto's thyroiditis, idiopathic myxedema, ovarian autoimmunity, autoimmune anti-sperm infertility, autoimmune prostatitis and Type I autoimmune polyglandular syndrome.

Non-limiting examples of inflammatory gastrointestinal diseases or disorders include colitis, ileitis, Crohn's disease, chronic inflammatory intestinal disease, inflammatory bowel syndrome, chronic inflammatory bowel disease, celiac disease, ulcerative colitis, an ulcer, a skin ulcer, a bed sore, a gastric ulcer, a peptic ulcer, a buccal ulcer, a nasopharyngeal ulcer, an esophageal ulcer, a duodenal ulcer and a gastrointestinal ulcer.

Non-limiting examples of inflammatory cutaneous diseases or disorders include acne, and an autoimmune bullous skin disease.

Non-limiting examples of inflammatory hepatic diseases or disorders include autoimmune hepatitis, hepatic cirrhosis, and biliary cirrhosis.

Non-limiting examples of inflammatory neurological diseases or disorders include multiple sclerosis, Alzheimer's disease, Parkinson's disease, myasthenia gravis, motor neuropathy, Guillain-Barre syndrome, autoimmune neuropathy, Lambert-Eaton myasthenic syndrome, paraneoplastic neurological disease or disorder, paraneoplastic cerebellar atrophy, non-paraneoplastic stiff man syndrome, progressive cerebellar atrophy, Rasmussen's encephalitis, amyotrophic lateral sclerosis, Sydeham chorea, Gilles de la Tourette syndrome, autoimmune polyendocrinopathy, dysimmune neuropathy, acquired neuromyotonia, arthrogryposis multiplex, Huntington's disease, AIDS associated dementia, amyotrophic lateral sclerosis (AML), multiple sclerosis, stroke, an inflammatory retinal disease or disorder, an inflammatory ocular disease or disorder, optic neuritis, spongiform encephalopathy, migraine, headache, cluster headache, and stiff-man syndrome.

Non-limiting examples of inflammatory connective tissue diseases or disorders include autoimmune myositis, primary Sjogren's syndrome, smooth muscle autoimmune disease or disorder, myositis, tendinitis, a ligament inflammation, chondritis, a joint inflammation, a synovial inflammation, carpal tunnel syndrome, arthritis, rheumatoid arthritis, osteoarthritis, ankylosing spondylitis, a skeletal inflammation, an autoimmune ear disease or disorder, and an autoimmune disease or disorder of the inner ear.

Non-limiting examples of inflammatory renal diseases or disorders include autoimmune interstitial nephritis and/or renal cancer.

Non-limiting examples of inflammatory reproductive diseases or disorders include repeated fetal loss, ovarian cyst, or a menstruation associated disease or disorder.

Non-limiting examples of inflammatory systemic diseases or disorders include systemic lupus erythematosus, systemic sclerosis, septic shock, toxic shock syndrome, and cachexia.

Non-limiting examples of infectious disease or disorder include chronic infectious diseases or disorders, a subacute infectious disease or disorder, an acute infectious disease or disorder, a viral disease or disorder, a bacterial disease or disorder, a protozoan disease or disorder, a parasitic disease or disorder, a fungal disease or disorder, a mycoplasma disease or disorder, gangrene, sepsis, a prion disease or disorder, influenza, tuberculosis, malaria, acquired immunodeficiency syndrome, and severe acute respiratory syndrome.

Non-limiting examples of inflammatory transplantation-related diseases or disorders include graft rejection, chronic graft rejection, subacute graft rejection, acute graft rejection hyperacute graft rejection, and graft versus host disease or disorder. Exemplary implants include a prosthetic implant, a breast implant, a silicone implant, a dental implant, a penile implant, a cardiac implant, an artificial joint, a bone fracture repair device, a bone replacement implant, a drug delivery implant, a catheter, a pacemaker, an artificial heart, an artificial heart valve, a drug release implant, an electrode, and a respirator tube.

Non-limiting examples of inflammatory tumors include a malignant tumor, a benign tumor, a solid tumor, a metastatic tumor and a non-solid tumor.

Non-limiting examples of inflammatory pulmonary diseases or disorders include asthma, allergic asthma, emphysema, chronic obstructive pulmonary disease or disorder, sarcoidosis and bronchitis.

Examples of a proliferative diseases or disorders include cancer, lymphoproliferative disorders, immunoproliferative disorders, myeloproliferative neoplasm and plasma cell proliferative disorder.

Cystic Fibrosis:

According to some embodiments of the present invention, there is provided a method of treating cystic fibrosis in a human subject (e.g., a human subject afflicted with cystic fibrosis, a human subject diagnosed with cystic fibrosis, or a human subject suffering form cystic fibrosis). Diagnosis of cystic fibrosis can be effected by methods known in the art, including the methods described in the Examples section that follows.

The method according to some embodiments comprises subjecting the human subject to intermittent inhalation of a gaseous mixture that comprises nitric oxide, as described in any one of the embodiments pertaining to intermittent inhalation, and any combination thereof.

According to embodiments of the present invention, the method of treating a human subject suffering from CF encompasses any beneficial therapeutic effect exhibited in a human subject diagnosed with, or suffering from CF, including, for example, amelioration of a symptom of CF (e.g., improvement of a pulmonary function), amelioration of a medical condition associated with CF (e.g., reduction of a microbial infection associated with CF, reduction of the load of a pathogenic microorganism which is associated with CF, reduction of inflammation), amelioration of an adverse effect caused by another treatment of CF, reduction of mortality in human subjects diagnosed with, or suffering from CF and general improvement of the medical and mental condition of a human subject diagnosed with, or suffering from CF.

In some embodiments, a method of treating CF as described herein is regarded as a method of treating a CF patient (e.g., a subject afflicted by cystic fibrosis, a subject diagnosed by cystic fibrosis), and encompasses a method of ameliorating a symptom of CF (e.g., improvement of a pulmonary function), ameliorating a medical condition associated with CF (e.g., treatment of a microbial infection associated with CF, reduction of the load of a pathogenic microorganism which is associated with CF, reduction of inflammation), ameliorating an adverse effect caused by another treatment of CF, prolonging the life time of a human subject diagnosed with, or suffering from CF, and/or generally improving a medical and/or mental condition of a human subject diagnosed with, or suffering from CF.

In terms of following the efficacy of the treatment of CF in a human subject diagnosed with, or suffering from CF, it is generally accepted that pulmonary function is one of the most simple and direct marker for alleviating the symptoms of CF, and hence that improvement of a pulmonary function is a human subject represents a beneficial treatment of a human subject diagnosed with, or suffering from CF.

One of the primary complications of CF is the accumulation of airway phlegm, which contains predominantly bacteria, inflammatory cells, polymeric DNA, and F-actin. The bacterial colonizations and infections are most often caused by Staphylococcus aureus, Pseudomonas aeruginosa, and Haemophilus influenzae. Escherichia coli and Klebsiella pneumoniae present as chronic colonization develop in the airways. Burkholderia cepacia has been isolated in older human subjects and is associated with a rapid decline in pulmonary function progressing to death.

Without being bound by any particular theory, it is assumed that nitric oxide, delivered in an exogenous gaseous form, easily enters the pulmonary system and acts by pulmonary vasodilatation, reducing bacterial load, reducing inflammation, and alleviating other clinical symptoms.

Nasal nitric oxide concentration has been found to be significantly lower in human subjects diagnosed with, or suffering from CF than in controls, and this reduced nitric oxide may play a role in bronchial obstruction and reduced defense to bacterial infections observed in human subjects diagnosed with, or suffering from CF.

In some embodiments, the method as described herein, in any one of the embodiments thereof, and in any combination thereof, is effected by improving one or more physiological parameters in a human subject diagnosed with, or suffering from CF which worsen by a medical condition associated with CF. An improvement of any of these parameters is indicative of the beneficial effect of the treatment by intermittent inhalation of nitric oxide, according to any one of the embodiments described herein.

According to some embodiments of the present invention, the method is effected by improving at least one pulmonary function (spirometric parameter), such as, but not limited to, Forced Expiratory Volume in 1 second (FEV), Forced Vital Capacity (FVC), FEV₁/FVC ratio or FEV₁% and Forced Expiratory Flow (FEF).

The spirometric parameter Forced Vital Capacity (FVC) is the volume of air measured in liters, which can forcibly be blown out after full inspiration, and constitutes the most basic maneuver in spirometry tests.

The spirometric parameter Forced Expiratory Volume in the 1st second (FEV1) is the volume of air that can forcibly be blown out in one second, after full inspiration. Average values for FEV₁ depend mainly on sex and age, whereas values falling between 80% and 120% of the average value are considered normal. Predicted normal values for FEV1 can be calculated on-site and depend on age, sex, height, weight and ethnicity as well as the research study that they are based on.

The spirometric parameter FEV₁/FVC ratio (FEV₁%) is the ratio of FEV₁ to FVC, which should be approximately 75-80%. The predicted FEV₁% is defined as FEV₁% of the patient divided by the average FEV₁% in the population appropriate for that patient.

The spirometric parameter Forced Expiratory Flow (FEF) is the flow (or speed) of air coming out of the lung during the middle portion of a forced expiration. It can be given at discrete times, generally defined by what fraction remains of the forced vital capacity (FVC), namely 25% of FVC (FEF₂₅), 50% of FVC (FEF₅₀) or 75% of FVC (FEF₇₅). It can also be given as a mean of the flow during an interval, also generally delimited by when specific fractions remain of FVC, usually 25-75% (FEF₂₅₋₇₅). Measured values ranging from 50-60% up to 130% of the average are considered normal, while predicted normal values for FEF can be calculated on-site and depend on age, sex, height, weight and ethnicity as well as the research study that they are based on. Recent research suggests that FEF₂₅₋₇₅% or FEF₂₅₋₅₀% may be a more sensitive parameter than FEV₁ in the detection of obstructive small airway disease. However, in the absence of concomitant changes in the standard markers, discrepancies in mid-range expiratory flow may not be specific enough to be useful, and current practice guidelines recommend continuing to use FEV₁, VC, and FEV₁/VC as indicators of obstructive disease.

It is noted that in some embodiments, other spirometric parameters, as these are defined and described herein below, may be used to follow the progression and efficacy of CF treatment by intermittent inhalation of 160 ppm nitric oxide, and/or to follow safety parameters of the treatment.

According to some embodiments, FEV₁ is monitored as an on-site parameter, as defined hereinafter, which is indicative of the beneficial effect of the intermittent inhalation of nitric oxide, as provided herewith. In general, an increase in the FEV₁ level is regarded as a desired effect in human subjects diagnosed with, or suffering from CF, wherein an increase of at least 3 percent in the FEV₁ baseline level of the patient (before commencing the treatment) is regarded as a notable improvement. In some embodiments, the method is effected such that FEV₁ level is increased by at least 3, 5, 10, 15 or 20 percent during and/or after the intermittent inhalation (e.g., during and/or after the entire time period intermittent inhalation of nitric oxide is effected) of nitric oxide, as described herein.

According to some embodiments of the present invention, the CF is associated with a microbial infection, that is, the human subject diagnosed with, or suffering from CF treated by a method as described herein suffers from a microbial infection. According to some embodiments of the present invention, the microbial infection is caused by one or more pathogenic microorganisms which can be for example, a Gram-negative bacterium, a Gram-positive bacterium, a virus and a viable virion, fungi and parasites.

According to some embodiments, the method of treating CF comprises treating a microbial infection associated with CF (a microbial infection that typically develops in a human subject diagnosed with, or suffering from CF), and/or reducing a load of a pathogenic microorganism that causes a microbial infection associated with CF (also referred to as a pathogenic microorganism associated with CF).

CF is typically associated with respiratory microbial infections caused by certain pathogens (pathogenic microorganisms associated with CF). These include, for example, P. alcaligenes, non-mucoid and mucoid Pseudomonas aeruginosa, A. fumigates, Staphylococcus aureus, Haemophilus influenza, Burkholderia cepacia complex, Klebsiella pneumonia, Escherichia coli, methicillin-resistant Staphylococcus aureus (MRSA), methicillin-sensitive Staphylococcus aureus (MSSA), Stenotrophomonas maltophilia, Achromobacter spp., Achromobacter xylosoxidans and non-tuberculous mycobacteria (NTM) species.

Such microbial infections can be regarded as a secondary condition to CF, or as an opportunistic infection in human subjects diagnosed with, or suffering from CF.

According to some embodiments, the pathogenic microorganism which is associated with CF is selected from the group consisting of P. alcaligenes, methicillin-sensitive Staphylococcus aureus (MSSA), Achromobacter spp., A. fumigates, non-mucoid P. aeruginosa and mucoid P. aeruginosa. According to some of these embodiments, the method as described herein comprises treating a microbial infection associated with CF and/or reducing the load of the pathogenic microorganism that causes the microbial infection (pathogenic microorganism associated with CF).

As demonstrated in the Examples section that follows below, the method presented herein has been demonstrated to reduce the load of several strains of pathogens known to cause debilitating and even fatal infections in human subjects diagnosed with, or suffering from CF.

According to embodiments of the present invention, the method is effected so as to reduce the load of the pathogenic microorganism in the subject by at least one log unit during the intermittent inhalation treatment.

The term “log unit” as used herein to describe a change in the load of a pathogenic microorganism, also known as “log reduction” or “log increase”, is a mathematical term used to show the relative number of live microbes eliminated from a system by carrying out the method of intermittent inhalation of nitric oxide, as presented herein. For example, a 5 log units reduction means lowering the number of microorganisms by 100,000-fold, that is, if a sample has 100,000 pathogenic microbes on it, a 5-log reduction would reduce the number of microorganisms to one. Hence, a 1 log unit reduction means the number of pathogenic microbes is 10 times smaller, a 2 log reduction means the number of pathogens is 100 times smaller, a 3 log reduction means the number of pathogens is 1000 times smaller, a 4 log reduction means the number of pathogens is 10,000 times smaller and so forth.

As known in the art, CF is typically associated with a state of inflammation in at least one bodily site, e.g. the lungs, or an acute, chronic, local or systemic inflammation, cause by one or more medical conditions, including but not limited to pathogenic infections. Inflammation in human subjects diagnosed with, or suffering from CF can also be regarded as a secondary condition to CF (a medical condition associated with CF). According to some embodiments of the present invention, the method is effected by reducing the level of inflammation associated with CF.

Reduction in inflammation associated with CF is typically regarded as a beneficial effect of the treatment of CF. Similarly, a reduction of a level of an inflammatory biomarker associated with CF can be regarded as an indication of efficacy of the method of treating a human subject diagnosed with, or suffering from CF as presented herein. For an exemplary provision of methods and discussion regarding the relationship of systemic inflammation to prior hospitalization in adult human subjects diagnosed with cystic fibrosis, see Ngan, D. A. et al., BMC Pulmonary Medicine, 2012, 12(3).

In the context of some embodiments of the present invention, inflammatory or inflammation biomarkers associated with CF include, without limitation, serum/blood levels of C-reactive protein (CRP), cytokines such as interleukins IL-6 and IL-1β, alpha-1-antitrypsin (AAT), haptoglobin, transferrin, various immunoglobulins, granzyme B (GzmB), chemokine C-C motif ligand 18 (CCL18/PARC), surfactant protein D (SP-D), lipopolysaccharide (LPS)-binding protein, and soluble cluster of differentiation 14 (sCD14).

The term “cytokine”, as used in the context of embodiments of the present invention, include chemokines, interferons, interleukins, lymphokines and tumor necrosis factor.

Following is a brief description of four non-limiting exemplary inflammatory biomarkers associated with CF.

Tumor Necrosis Factor alpha (TNFα) signals to the body to bring the neutrophil white blood cells to the site of infection or injury. TNFα is known as a cytokine, or a cell-signaling protein. TNFα acts like a “first responder” at an accident by signaling to the body where the most damage is so that the immune system can respond effectively, which is to send neutrophils.

Nuclear Factor kappa B (NFkB) is a transcription factor protein complex that acts as a switch for certain genes. When NFkB is allowed to enter the nucleus, which it does through the aid of TNFα, it turns on the genes which allow cells to proliferate, mature, and avoid destruction through apoptosis (programmed cell death). This allows white blood cells to replicate and effect their activity in cleaning up the infected or injured area. NFkB is similar to the priority setting on a communications line by opening all channels available for the quickest response.

Interleukin-6 (IL-6) is a cytokine that dictates the neutrophils to destroy themselves and draws monocytes, another type of white blood cell, to the infected or injured area instead. The monocytes create macrophages which clean up the debris and pathogens through phagocytosis, the process by which macrophages degrade dead cells and other particles whole.

C-Reactive Protein (CRP) is a “pattern recognition receptor” protein, which means it marks recognized debris for removal, that is produced by the liver in response to IL-6 levels and binds to the surface of dead and dying cells, and also to certain forms of bacteria. CRP acts as a form of signal for the macrophages to ingest something through phagocytosis, and thus helps in the ultimate clearing of debris during inflammation.

According to some embodiments, monitoring the level of an inflammatory biomarker associated with CF is useful in determining the course and effect of the treatment of inflammation associated with CF. In some embodiments, the level of a biomarker associated with CF in the serum extracted from the subject, based on a baseline of the serum level in the subject before commencement of the treatment, is reduced by at least 3, 5, 10, 15, 20, 30, 35, 40, 50 or at least 60 percent during the treatment.

In some embodiment, the biomarker associated with CF is CRP, and the serum level of CRP is reduced during the intermittent inhalation treatment by at least 3, 5, 10, 15, 20, 30, 35, 40, 50 or at least 60 percent, compared to the baseline level in the subject before commencement of the treatment.

In some embodiment, the biomarker associated with CF is a cytokine, such as, but not limited to, TNFα, IL-1β, IL-6, IL-8, IL-10 and/or IL-12p70, and the serum level of the cytokine(s) is reduced by at least 3, 5, 10, 15, 20, 30, 35, 40, 50 or at least 60 percent, compared to the baseline level in the subject before commencement of the treatment. In some embodiments, the cytokines used as inflammatory biomarkers in the method presented herein are IL-6 and IL-1β.

According to some embodiments of the present invention, there is provided a method of reducing a load of a pathogenic microorganism in a human subject by subjecting the human subject to intermittent inhalation of a gas mixture comprising nitric oxide at a concentration of at least 160 ppm.

According to some embodiments of this aspect, the human subject is a human subject diagnosed with, or suffering from CF, as described herein.

In some embodiments, the pathogenic microorganism causes a microbial infection associated with CF, as described herein. According to some embodiments, the pathogenic microorganism is selected from the group consisting of P. alcaligenes, non-mucoid and mucoid Pseudomonas aeruginosa, A. fumigates, Staphylococcus aureus, Haemophilus influenza, Burkholderia cepacia complex, Klebsiella pneumonia, Escherichia coli, methicillin-resistant Staphylococcus aureus (MRSA), methicillin-sensitive Staphylococcus aureus (MSSA), Stenotrophomonas maltophilia, Achromobacter spp., Achromobacter xylosoxidans and non-tuberculous mycobacteria (NTM) species.

According to some embodiments, the pathogenic microorganism is selected from the group consisting of P. alcaligenes, methicillin-sensitive Staphylococcus aureus (MSSA), Achromobacter spp., A. fumigates, non-mucoid P. aeruginosa and mucoid P. aeruginosa.

As discussed hereinabove, in some embodiments, the load of the pathogenic microorganism is reduced by the presently claimed method by at least 1 log units during the intermittent inhalation.

According to some embodiments of the present invention, there is provided a method of reducing a level of an inflammatory biomarker associated with CF in a human subject by subjecting the human subject to a treatment by intermittent inhalation of a gas mixture comprising nitric oxide at a concentration of at least 160 ppm.

According to some embodiments, the inflammatory biomarker associated with CF, and/or a change in its normal physiological level, is associated with cystic fibrosis and/or with complications and other medical conditions associated with CF. Reducing a level of an inflammatory biomarker associated with CF in a human subject diagnosed with, or suffering from CF is indicative of treating inflammation (as a secondary medical condition) in a human subject diagnosed with, or suffering from CF.

According to some embodiments, the inflammatory biomarker, which is targeted for reduction by the presently claimed method is selected from the group consisting of C-reactive protein (CRP), a cytokine, alpha-1-antitrypsin (AAT), haptoglobin, transferrin, an immunoglobulin, granzyme B (GzmB), chemokine C-C motif ligand 18 (CCL18/PARC), surfactant protein D (SP-D), lipopolysaccharide (LPS)-binding protein and soluble cluster of differentiation 14 (sCD14).

In some embodiments of this aspect of the present invention, the inflammatory biomarker associated with CF is C-reactive protein (CRP). A rate of reduction as a result of the intermittent inhalation is at least 3, 5, 10, 15, 20, 30, 35, 40, 50 or at least 60 percent, compared to a baseline level of the biomarker in the patient.

In some embodiments of this aspect of the present invention, the inflammatory biomarker associated with CF is a cytokine is selected from the group consisting of TNFα, IL-1β, IL-6, IL-8, IL-10 and IL-12p70. In some embodiments, the inflammatory biomarkers are IL-6 and IL-10. A rate of reduction in the level of a cytokine as a result of the treatment is at least 3, 5, 10, 15, 20, 30, 35, 40, 50 or at least 60 percent, compared to a baseline level of the biomarker in the patient.

According to some embodiments of this aspect, the human subject is a cystic fibrosis patient, as described herein.

Bronchiolitis:

According to some embodiments of the present invention, there is provided a method of treating bronchiolitis in a subject in need thereof (e.g., a subject afflicted with bronchiolitis, a subject diagnosed with bronchiolitis). Diagnosis of Cystic fibrosis can be effected by methods known in the art, including the methods described in the Examples section that follows.

The method as described herein comprises subjecting the human subject to intermittent inhalation of a gaseous mixture that comprises nitric oxide, as described in any one of the embodiments pertaining to intermittent inhalation, and any combination thereof.

According to embodiments of the present invention, the method of treating bronchiolitis encompasses any beneficial therapeutic effect exhibited in a bronchiolitis patient, including, for example, amelioration of a symptom of bronchiolitis (e.g., improvement of a pulmonary function), amelioration of a medical condition associated with bronchiolitis (e.g., reduction of a microbial infection associated with bronchiolitis, reduction of the load of a pathogenic microorganism which is associated with bronchiolitis, reduction of inflammation), and reduction in the length of hospitalization of a patient.

In some embodiments, a method of treating bronchiolitis as described herein is regarded as a method of treating a subject suffering from bronchiolitis, and encompasses a method of ameliorating a symptom of bronchiolitis (e.g., improvement of a pulmonary function), amelioration of a medical condition associated with bronchiolitis (e.g., reduction of a microbial infection associated with bronchiolitis, reduction of the load of a pathogenic microorganism which is associated with bronchiolitis, reduction of inflammation), and reduction in the length of hospitalization of a patient.

In terms of following the efficacy of the treatment of bronchiolitis in a human, it is generally accepted that pulmonary function is one of the most simple and direct marker for alleviating the symptoms of bronchiolitis, and hence that improvement of a pulmonary function is a human subject represents a beneficial treatment of a human subject suffering from bronchiolitis.

Without being bound by any particular theory, it is assumed that nitric oxide, delivered in an exogenous gaseous form, easily enters the pulmonary system and acts by pulmonary vasodilatation, reducing pathogenic microbial load, reducing inflammation, and alleviating other clinical symptoms.

In some embodiments, the method as described herein, in any one of the embodiments thereof, and in any combination thereof, is effected by improving one or more physiological parameters in a subject suffering from bronchiolitis which worsen by a medical condition associated with bronchiolitis. An improvement of any of these parameters is indicative of the beneficial effect of the treatment by intermittent inhalation of nitric oxide, according to any one of the embodiments described herein.

According to some embodiments of the present invention, the method is effected by improving at least one pulmonary function (spirometric parameter), such as, but not limited to, Forced Expiratory Volume in 1 second (FEV), Forced Vital Capacity (FVC), FEV₁/FVC ratio or FEV₁% and Forced Expiratory Flow (FEF).

The spirometric parameter Forced Vital Capacity (FVC) is the volume of air measured in liters, which can forcibly be blown out after full inspiration, and constitutes the most basic maneuver in spirometry tests.

The spirometric parameter Forced Expiratory Volume in the 1st second (FEV₁) is the volume of air that can forcibly be blown out in one second, after full inspiration. Average values for FEV₁ depend mainly on sex and age, whereas values falling between 80% and 120% of the average value are considered normal. Predicted normal values for FEV₁ can be calculated on-site and depend on age, sex, height, weight and ethnicity as well as the research study that they are based on.

The spirometric parameter FEV₁/FVC ratio (FEV₁%) is the ratio of FEV₁ to FVC, which should be approximately 75-80%. The predicted FEV₁% is defined as FEV₁% of the patient divided by the average FEV₁% in the population appropriate for that patient.

The spirometric parameter Forced Expiratory Flow (FEF) is the flow (or speed) of air coming out of the lung during the middle portion of a forced expiration. It can be given at discrete times, generally defined by what fraction remains of the forced vital capacity (FVC), namely 25% of FVC (FEF₂₅), 50% of FVC (FEF₅₀) or 75% of FVC (FEF₇₅). It can also be given as a mean of the flow during an interval, also generally delimited by when specific fractions remain of FVC, usually 25-75% (FEF₂₅₋₇₅). Measured values ranging from 50-60% up to 130% of the average are considered normal, while predicted normal values for FEF can be calculated on-site and depend on age, sex, height, weight and ethnicity as well as the research study that they are based on. Recent research suggests that FEF₂₅₋₇₅% or FEF₂₅₋₅₀% may be a more sensitive parameter than FEV₁ in the detection of obstructive small airway disease. However, in the absence of concomitant changes in the standard markers, discrepancies in mid-range expiratory flow may not be specific enough to be useful, and current practice guidelines recommend continuing to use FEV₁, VC, and FEV₁/VC as indicators of obstructive disease.

It is noted that in some embodiments, other spirometric parameters, as these are defined and described herein below, may be used to follow the progression and efficacy of bronchiolitis treatment by intermittent inhalation of 160 ppm nitric oxide, and/or to follow safety parameters of the treatment.

According to some embodiments, FEV₁ is monitored as an on-site parameter, as defined hereinafter, which is indicative of the beneficial effect of the intermittent inhalation of nitric oxide, as provided herewith. In general, an increase in the FEV₁ level is regarded as a desired effect in subjects suffering from bronchiolitis, wherein an increase of at least 3 percent in the FEV₁ baseline level of the patient (before commencing the treatment) is regarded as a notable improvement. In some embodiments, the method is effected such that FEV₁ level is increased by at least 3, 5, 10, 15 or 20 percent during and/or after the intermittent inhalation (e.g., during and/or after the entire time period intermittent inhalation of nitric oxide is effected) of nitric oxide, as described herein.

As demonstrated in the Examples section that follows below, the method presented herein has been demonstrated to reduce the load of several strains of pathogens known to cause debilitating and even fatal infections in subjects suffering from bronchiolitis.

According to embodiments of the present invention, the method is effected so as to reduce the load of the pathogenic microorganism in the subject by at least one log unit during the intermittent inhalation treatment.

The term “log unit” as used herein to describe a change in the load of a pathogenic microorganism, also known as “log reduction” or “log increase”, is a mathematical term used to show the relative number of live microbes eliminated from a system by carrying out the method of intermittent inhalation of nitric oxide, as presented herein. For example, a 5 log units reduction means lowering the number of microorganisms by 100,000-fold, that is, if a sample has 100,000 pathogenic microbes on it, a 5-log reduction would reduce the number of microorganisms to one. Hence, a 1 log unit reduction means the number of pathogenic microbes is 10 times smaller, a 2 log reduction means the number of pathogens is 100 times smaller, a 3 log reduction means the number of pathogens is 1000 times smaller, a 4 log reduction means the number of pathogens is 10,000 times smaller and so forth.

Bronchiolitisis typically associated with a state of inflammation in at least one bodily site, e.g. the lungs, or an acute, chronic, local or systemic inflammation, cause by one or more medical conditions, including but not limited to pathogenic infections. Inflammation in a subject suffering from bronchiolitis can also be regarded as a secondary condition to bronchiolitis (a medical condition associated with bronchiolitis). According to some embodiments of the present invention, the method is effected by reducing the level of inflammation associated with bronchiolitis.

Reduction in inflammation associated with bronchiolitis is typically regarded as a beneficial effect of the treatment of bronchiolitis. Similarly, a reduction of a level of an inflammatory biomarker associated with bronchiolitis can be regarded as an indication of efficacy of the method of treating a subject suffering from bronchiolitis as presented herein.

In the context of some embodiments of the present invention, inflammatory or inflammation biomarkers associated with bronchiolitis include, without limitation, serum/blood levels of C-reactive protein (CRP), cytokines such as interleukins IL-6 and IL-1β, alpha-1-antitrypsin (AAT), haptoglobin, transferrin, various immunoglobulins, granzyme B (GzmB), chemokine C-C motif ligand 18 (CCL18/PARC), surfactant protein D (SP-D), lipopolysaccharide (LPS)-binding protein, and soluble cluster of differentiation 14 (sCD14).

The term “cytokine”, as used in the context of embodiments of the present invention, include chemokines, interferons, interleukins, lymphokines and tumor necrosis factor.

Following is a brief description of four non-limiting exemplary inflammatory biomarkers associated with bronchiolitis.

Tumor Necrosis Factor alpha (TNFα) signals to the body to bring the neutrophil white blood cells to the site of infection or injury. TNFα is known as a cytokine, or a cell-signaling protein. TNFα acts like a “first responder” at an accident by signaling to the body where the most damage is so that the immune system can respond effectively, which is to send neutrophils.

Nuclear Factor kappa B (NFkB) is a transcription factor protein complex that acts as a switch for certain genes. When NFkB is allowed to enter the nucleus, which it does through the aid of TNFα, it turns on the genes which allow cells to proliferate, mature, and avoid destruction through apoptosis (programmed cell death). This allows white blood cells to replicate and effect their activity in cleaning up the infected or injured area. NFkB is similar to the priority setting on a communications line by opening all channels available for the quickest response.

Interleukin-6 (IL-6) is a cytokine that dictates the neutrophils to destroy themselves and draws monocytes, another type of white blood cell, to the infected or injured area instead. The monocytes create macrophages which clean up the debris and pathogens through phagocytosis, the process by which macrophages degrade dead cells and other particles whole.

C-Reactive Protein (CRP) is a “pattern recognition receptor” protein, which means it marks recognized debris for removal, that is produced by the liver in response to IL-6 levels and binds to the surface of dead and dying cells, and also to certain forms of bacteria. CRP acts as a form of signal for the macrophages to ingest something through phagocytosis, and thus helps in the ultimate clearing of debris during inflammation.

According to some embodiments, monitoring the level of an inflammatory biomarker associated with bronchiolitis is useful in determining the course and effect of the treatment of inflammation associated with bronchiolitis. In some embodiments, the level of a biomarker associated with bronchiolitis in the serum extracted from the subject, based on a baseline of the serum level in the subject before commencement of the treatment, is reduced by at least 3, 5, 10, 15, 20, 30, 35, 40, 50 or at least 60 percent during the treatment.

In some embodiment, the biomarker associated with bronchiolitis is CRP, and the serum level of CRP is reduced during the intermittent inhalation treatment by at least 3, 5, 10, 15, 20, 30, 35, 40, 50 or at least 60 percent, compared to the baseline level in the subject before commencement of the treatment.

In some embodiment, the biomarker associated with bronchiolitis is a cytokine, such as, but not limited to, TNFα, IL-10, IL-6, IL-8, IL-10 and/or IL-12p70, and the serum level of the cytokine(s) is reduced by at least 3, 5, 10, 15, 20, 30, 35, 40, 50 or at least 60 percent, compared to the baseline level in the subject before commencement of the treatment. In some embodiments, the cytokines used as inflammatory biomarkers in the method presented herein are IL-6 and IL-1β.

According to some embodiments of the present invention, there is provided a method of reducing a load of a pathogenic microorganism in a human subject by subjecting the human subject to intermittent inhalation of a gas mixture comprising nitric oxide at a concentration of at least 160 ppm.

According to some embodiments of this aspect, the human subject is a subject suffering from bronchiolitis, as described herein.

In some embodiments, the pathogenic microorganism causes a microbial infection associated with bronchiolitis, as described herein. According to some embodiments, the pathogenic microorganism is selected from the group consisting of P. alcaligenes, non-mucoid and mucoid Pseudomonas aeruginosa, A. fumigates, Staphylococcus aureus, Haemophilus influenza, Burkholderia cepacia complex, Klebsiella pneumonia, Escherichia coli, methicillin-resistant Staphylococcus aureus (MRSA), methicillin-sensitive Staphylococcus aureus (MSSA), Stenotrophomonas maltophilia, Achromobacter spp., Achromobacter xylosoxidans and non-tuberculous mycobacteria (NTM) species.

According to some embodiments, the pathogenic microorganism is selected from the group consisting of P. alcaligenes, methicillin-sensitive Staphylococcus aureus (MSSA), Achromobacter spp., A. fumigates, non-mucoid P. aeruginosa and mucoid P. aeruginosa.

As discussed hereinabove, in some embodiments, the load of the pathogenic microorganism is reduced by the presently claimed method by at least 1 log units during the intermittent inhalation.

According to some embodiments of the present invention, there is provided a method of reducing a level of an inflammatory biomarker associated with bronchiolitis in a human subject by subjecting the human subject to a treatment by intermittent inhalation of a gas mixture comprising nitric oxide at a concentration of at least 160 ppm.

According to some embodiments, the inflammatory biomarker associated with bronchiolitis, and/or a change in its normal physiological level, is associated with cystic fibrosis and/or with complications and other medical conditions associated with bronchiolitis. Reducing a level of an inflammatory biomarker associated with bronchiolitis in a subject suffering from bronchiolitis is indicative of treating inflammation (as a secondary medical condition).

According to some embodiments, the inflammatory biomarker associated with bronchiolitis, which is targeted for reduction by the presently claimed method is selected from the group consisting of C-reactive protein (CRP), a cytokine, alpha-1-antitrypsin (AAT), haptoglobin, transferrin, an immunoglobulin, granzyme B (GzmB), chemokine C-C motif ligand 18 (CCL18/PARC), surfactant protein D (SP-D), lipopolysaccharide (LPS)-binding protein and soluble cluster of differentiation 14 (sCD14).

In some embodiments of this aspect of the present invention, the inflammatory biomarker associated with bronchiolitis is C-reactive protein (CRP). A rate of reduction as a result of the intermittent inhalation is at least 3, 5, 10, 15, 20, 30, 35, 40, 50 or at least 60 percent, compared to a baseline level of the biomarker in the patient.

In some embodiments of this aspect of the present invention, the inflammatory biomarker associated with bronchiolitis is a cytokine is selected from the group consisting of TNFα, IL-1β, IL-6, IL-8, IL-10 and IL-12p70. In some embodiments, the inflammatory biomarkers are IL-6 and IL-1β. A rate of reduction in the level of a cytokine as a result of the treatment is at least 3, 5, 10, 15, 20, 30, 35, 40, 50 or at least 60 percent, compared to a baseline level of the biomarker in the patient.

According to some embodiments of this aspect, the human subject is a cystic fibrosis patient, as described herein.

Intermittent Inhalation:

As presented hereinabove, any of the methods provided herewith comprise subjecting the human subject to intermittent inhalation of a gas mixture comprising nitric oxide at a concentration of at least 160 ppm.

The term “intermittent” is used herein and in the art as an antonym of “continuous”, and means starting and ceasing an action and/or performing an action in intervals.

By “intermittent inhalation” it is meant that a human subject breathes a mixture of gases that contains an indicated concentration of nitric oxide intermittently; hence while the volume of the inhaled mixture of gases may not change significantly during the intermittent inhalation, the chemical composition of the mixture changes according to a predetermined regimen, as described herein below. The human subject therefore inhales a gas mixture comprising nitric oxide at a concentration of at least 160 ppm for predetermined periods of time, and between these periods of time the human subject inhales a gaseous mixture that is essentially devoid of nitric oxide (e.g., ambient air or another nitric oxide-free mixture).

Herein and throughout, “a nitric oxide-containing gaseous mixture” or “a gas mixture comprising nitric oxide” is used to describe a gaseous mixture that contains at least 160 ppm nitric oxide. The nitric oxide-containing mixture can comprise 160 ppm, 170 ppm, 180 ppm, 190 ppm, 200 ppm and even higher concentrations of nitric oxide. Other gaseous mixtures mentioned herein include less than 160 ppm nitric oxide or are being essentially devoid of nitric oxide, as defined herein.

By “essentially devoid of nitric oxide” it is meant no more than 50 ppm, no more than 40 ppm, no more than 30 ppm, no more than 20 ppm, no more than 10 ppm, no more than 5 ppm, no more than 1 ppm and no more than ppb, including absolutely no nitric oxide.

According to some embodiments of the present invention, the intermittent inhalation includes one or more cycles, each cycle comprising continuous inhalation of a gaseous mixture containing nitric oxide at the specified high concentration (e.g., at least 160 ppm) for a first time period, followed by inhalation of a gaseous mixture essentially devoid of nitric oxide for a second time period. According to some embodiments of the present invention, during the second period of time the subject may inhale ambient air or a controlled mixture of gases, which is essentially devoid of nitric oxide, as defined herein.

In some embodiments, the first time period spans from 10 minutes to 45 minutes, or from 20 to 45 minutes, or from 20 to 40 minutes, and according to some embodiments, spans about 30 minutes.

According to some embodiments of the present invention, the second time period ranges from 3 hours to 5 hours, or from 3 to 4 hours, and according to some embodiments the second time period spans about 3.5 hours.

According to some embodiments of the present invention, this inhalation regimen is repeated 1-6 times over 24 hours, depending on the duration of the first and second time periods.

In some embodiments, a cycle of intermittent delivery of nitric oxide, e.g., 160 ppm for 30 minutes followed by 3.5 hours of breathing no nitric oxide, is repeated from 1 to 6 times a day. According to some embodiments, the cycles are repeated 5 times a day. Alternatively the cycles are repeated 3 times a day.

According to some embodiments of the present invention, the regimen of 1-5 cycles per day is carried out for 1 to 21 days, or from 2 to 14 days, or from 3 to 10 days. According to some embodiments of the present invention, the intermittent inhalation is effected during a time period of 2 weeks. However, longer time periods of intermittent nitric oxide administration as described herein, are also contemplated.

Safety:

As discussed hereinabove, intermittent inhalation of 160 ppm of nitric oxide has been shown to be safe in human subjects of all ages. Safety has been demonstrated by monitoring one or more physiological parameters in the human and while minding that no substantial adverse change is effected in the monitored parameters, as a safety measure of the method presented herein. According to any one of the embodiments of the present invention, the intermittent inhalation is effected while monitoring one or more physiological parameters in the human subject.

In some embodiments, the methods disclosed herein are effected while monitoring various parameters relevant for maintaining the desired dosage and regimen, relevant to the safety of the procedure and relevant for efficacy of the treatment.

According to any one of the embodiments of the present invention, the method is effected while monitoring one or more physiological parameters in the human and while minding that no substantial adverse change is effected in the monitored safety parameters, as a safety measure of the method presented herein.

In some embodiments, the method is carried out while maintaining safety measured which include non-invasive monitoring of bodily fluid chemistry, such as perfusion index (PI), respiration rate (RRa), oxyhemoglobin saturation (SpO₂/SaO₂/DO), total hemoglobin (SpHb), carboxyhemoglobin (SpCO), methemoglobin (SpMet), oxygen content (SpOC), and pleth variability index (PVI), as these physiological parameters are known in the art. Typically, these on-site physiological parameters are monitored by pulse oximetry.

Other parameters, also monitored as a safety measure on the presently disclosed method, according to some embodiments thereof, are off-site physiological parameters which are typically determined by collecting bodily samples using non-invasive (e.g., urine, feces or sputum samples) and invasive (e.g., blood or biopsy) method.

For example, off-site physiological parameters which are typically measured by invasive methods may include serum nitrite/nitrate (NO₂⁻/NO₃⁻), blood methemoglobin, a complete blood cells count (CBC), blood chemistry/biochemistry (electrolytes, renal and liver function tests etc.) and coagulation tests.

Off-site physiological parameters which are typically measured by non-invasive methods may include urine nitrite/nitrate (NO₂⁻/NO₃⁻), pregnancy tests in urine, and bacterial and fungal load in sputum, urine or feces.

In some embodiments, the method is carried out while maintaining safety measures which include controlling the mixture of inhaled gases and monitoring the exhaled gases, which is effected by standard means for monitoring and controlling, on-site, the contents and/or flow of the mixture to which the subject is subjected to, or that which is delivered through a delivery interface, and/or while monitoring on-site exhaled gases and controlling the intake by feedback in real-time. In some embodiments, the method is effected while monitoring the concentration of nitric oxide, O₂, CO₂ and NO₂ in the gaseous mixture to which the human is exposed to or exhales

In some embodiments, the concentration of nitric oxide in the nitric oxide-containing gaseous mixture is controlled so as not to deviate from a predetermined concentration by more than 10%. For example, the method is carried out while the concentration of nitric oxide, set to 160 ppm, does not exceed substantially the margins of 144 ppm to 176 ppm.

Similarly, the NO₂ content in a nitric oxide-containing gaseous mixture is controlled such that the concentration of NO₂ is maintained lower than 5 ppm.

Further, oxygen level in the nitric oxide-containing gaseous mixture is controlled such that the concentration of O₂ in the mixture ranges from about 20% to about 25%.

Alternatively or in addition, the oxygen level in the nitric oxide-containing gaseous mixture is controlled such that the fraction of inspired oxygen (FiO₂) ranges from about 20% to about 100%.

The phrase “fraction of inspired oxygen” or “FiO₂”, as used herein, refers to the fraction or percentage of oxygen in a given gas sample. For example, ambient air at sea level includes 20.9% oxygen, which is equivalent to FiO₂ of 0.21. Oxygen-enriched air has a higher FiO₂ than 0.21, up to 1.00, which means 100% oxygen. In the context of embodiments of the present invention, FiO₂ is kept under 1 (less than 100% oxygen).

According to some embodiments, fraction of inspired oxygen (FiO₂) in the nitric oxide-containing gaseous mixture is 0.20. In an alternate embodiment, the FiO₂ in the nitric oxide-containing gaseous mixture is 0.25. In an alternate embodiment, the FiO₂ in the nitric oxide-containing gaseous mixture is 0.3. In an alternate embodiment, the FiO₂ in the nitric oxide-containing gaseous mixture is 0.35. In an alternate embodiment, the FiO₂ in the nitric oxide-containing gaseous mixture is 0.4. In an alternate embodiment, the FiO₂ in the nitric oxide-containing gaseous mixture is 0.45. In an alternate embodiment, the FiO₂ in the nitric oxide-containing gaseous mixture is 0.5. In an alternate embodiment, the FiO₂ in the nitric oxide-containing gaseous mixture is 0.55. In an alternate embodiment, the FiO₂ in the nitric oxide-containing gaseous mixture is 0.6. In an alternate embodiment, the FiO₂ in the nitric oxide-containing gaseous mixture is 0.65. In an alternate embodiment, the FiO₂ in the nitric oxide-containing gaseous mixture is 0.7. In an alternate embodiment, the FiO₂ in the nitric oxide-containing gaseous mixture is 0.75. In an alternate embodiment, the FiO₂ in the nitric oxide-containing gaseous mixture is 0.8. In an alternate embodiment, the FiO₂ in the nitric oxide-containing gaseous mixture is 0.85. In an alternate embodiment, the FiO₂ in the nitric oxide-containing gaseous mixture is 0.9. In an alternate embodiment, the FiO₂ in the nitric oxide-containing gaseous mixture is 0.95.

In some embodiments, the nitric oxide-containing gaseous mixture is formed by combining a stock supply of nitric oxide with air, which dilutes the stock supply of nitric oxide to the desired concentration. In some embodiments, the stock supply of nitric oxide is combined with air and oxygen to keep the FiO₂ above 0.20. The ratio of nitric oxide, air and/or oxygen can be varied to achieve the desired nitric oxide concentration and FiO₂.

The phrase “end tidal CO₂” or “ETCO₂”, as used herein, refers to the partial pressure or maximal concentration of carbon dioxide (CO₂) at the end of an exhaled breath, which is expressed as a percentage of CO₂ or the pressure unit mmHg. Normal values for humans range from 5% to 6% CO₂, which is equivalent to 35-45 mmHg. Since CO₂ diffuses out of the lungs into the exhaled air, ETCO₂ values reflect cardiac output (CO) and pulmonary blood flow as the gas is transported by the venous system to the right side of the heart and then pumped to the lungs by the right ventricles. A device called capnometer measures the partial pressure or maximal concentration of CO₂ at the end of exhalation. In the context of embodiments of the present invention, a capnometer is used and ETCO₂ levels are monitored so as to afford a warning feedback when ETCO₂ is more than 60 mmHg.

Levels of respiratory NO, NO₂ and O₂ concentration levels (both inhaled and exhaled; inspiratory and expiratory gases) are typically monitored continuously by sampling from a mouthpiece sample port located in an inhalation mask NO, NO₂ and O₂ equipped with an electrochemical analyzer. In the context of embodiments of the present invention, safety considerations requires the absolute minimization of the number of occasions in which NO₂ levels exceed 5 ppm, nitric oxide concentration variations exceeding 10%, and FiO₂/O₂ levels drop below 20% during nitric oxide administration.

It is noted that a sharp elevation of inflammatory biomarkers may be associated with a phenomenon called “cytokine storm”, which has been observed in subjects undergoing nitric oxide inhalation treatment. Hence, monitoring inflammatory biomarkers while performing the method as described herein has an additional role in safety considerations pertaining to the method, according to embodiments of the present invention, wherein no significant increase in inflammatory markers is an indication of safety.

In some embodiments, monitoring the one or more physiological parameters is effected by noninvasive measures and/or mild invasive measures.

In some embodiments, monitoring the physiological parameter(s) in the subject is effected by on-site measurement and analysis techniques based on samples collected sporadically, continuously or periodically from the subject on-site in real-time at the subject's bed-side, and/or off-site measurement and analysis techniques based on samples collected sporadically or periodically from the subject which are sent for processing in a off-site which provides the results and analysis at a later point in time.

In the context of some embodiments of the present invention, the phrase “on-site measurement and analysis techniques” or “on-site techniques”, refers to monitoring techniques that inform the practitioner of a given physiological parameter of the subject in real-time, without the need to send the sample or raw data to an off-site facility for analysis. On-site techniques are often noninvasive, however, some rely on sampling from an invasive medical device such as a respiratory tubus, a drainer tube, an intravenous catheter or a subcutaneous port or any other implantable probe. Thus, the phrase “on-site parameters”, as used herein, refers to physiological parameters which are obtainable by online techniques.

Other than the trivial advantage of real-time on-site determination of physiological parameters, expressed mostly in the ability of a practitioner to respond immediately and manually to any critical change thereof, the data resulting from real-time online determination of physiological parameters can be fed into the machinery and be used for real-time feedback controlling of the machinery. In the context of embodiments of the present invention, the term “real-time” also relates to systems that update information and respond thereto substantially at the same rate they receive the information. Such real-time feedback can be used to adhere to the treatment regimen and/or act immediately and automatically in response to any critical deviations from acceptable parameters as a safety measure.

Hence, according to embodiments of the present invention, the term “on-site parameter” refers to physiological and/or mechanical and/or chemical datum which is obtainable and can be put to use or consideration at or near the subject's site (e.g., bed-side) in a relatively short period of time, namely that the time period spanning the steps of sampling, testing, processing and displaying/using the datum is relatively short. An “on-site parameter” can be obtainable, for example, in less than 30 minutes, less than 10 minutes, less than 5 minutes, less than 1 minute, less than 0.5 minutes, less than 20 seconds, less than 10 seconds, less than 5 seconds, or less than 1 second from sampling to use. For example, the time period required to obtain on-site parameters by a technique known as pulse oximetry is almost instantaneous; once the device is in place and set up, data concerning, e.g., oxygen saturation in the periphery of a human subject, are available in less than 1 second from sampling to use.

In the context of some embodiments of the present invention, the phrase “off-site measurement and analysis techniques” or “off-site techniques”, refers to techniques that provide information regarding a given physiological parameter of the subject after sending a sample or raw data to an offline, and typically off-site facility, and receiving the analysis offline, sometimes hours or days after the sample had been obtained. Off-site techniques are oftentimes based on samples collected by mild invasive techniques, such as blood extraction for monitoring inflammatory cytokine plasma level, and invasive techniques, such as biopsy, catheters or drainer tubus, however, some off-site techniques rely on noninvasive sampling such as urine and stool chemistry offline and off-site analyses. The phrase “off-site parameters”, as used herein, refers to physiological parameters which are obtainable by off-site laboratory techniques.

Hence, according to embodiments of the present invention, the term “off-site parameter” refers to physiological and/or mechanical and/or chemical datum which is obtain and can be put to use or consideration in a relatively long period of time, namely that the time period spanning the steps of sampling, testing, processing and displaying/using the datum is long compared to on-site parameters. Thus, an “off-site parameter” is obtainable in more than 1 day, more than 12 hours, more than 1 hour, more than 30 minutes, more than 10 minutes, or more than 5 minutes from sampling to use.

An “off-site parameter” is typically obtainable upon subjecting a sample to chemical, biological, mechanical or other procedures, which are typically performed in a laboratory and hence are not performed “on-site”, namely by or near the subject's site.

Noninvasive measures for monitoring various physiological parameters include, without limitation, sputum, urine and feces sampling, pulse oximetry, nonintubated respiratory analysis and/or capnometry. Invasive measures for monitoring various physiological parameters include, without limitation, blood extraction, continuous blood gas and metabolite analysis, and in some embodiments intubated respiratory analysis and transcutaneous monitoring measures. Intense invasive measures include biopsy and other surgical procedures.

The term “pulse oximetry” refers to a noninvasive and on-site technology that measures respiration-related physiological parameters by following light absorption characteristics of hemoglobin through the skin (finger, ear lobe etc.), and on the spectroscopic differences observed in oxygenated and deoxygenated species of hemoglobin, as well as hemoglobin species bound to other molecules, such as carbon monoxide (CO), and methemoglobin wherein the iron in the heme group is in the Fe³⁺ (ferric) state. Physiological parameters that can be determined by pulse oximetry include, for example, SpO₂, SpMet and SpCO.

The phrase “nonintubated respiratory analysis”, as used herein, refers to a group of noninvasive and on-site technologies, such as spirometry and capnography, which provide measurements of the physiological pulmonary mechanics and respiratory gaseous chemistry by sampling the inhaled/exhaled airflow or by directing subject's breath to a detector, all without entering the subject's respiratory tract or other orifices nor penetrating the skin at any stage.

The term “spirometry” as used herein, refers to the battery of measurements of respiration-related parameters and pulmonary functions by means of a noninvasive and on-site spirometer. Following are exemplary spirometry parameters which may be used in the context of some embodiments of the present invention:

The spirometric parameter Tidal volume (TV) is the amount of air inhaled and exhaled normally at rest, wherein normal values are based on person's ideal body weight.

The spirometric parameter Total Lung Capacity (TLC) is the maximum volume of air present in the lungs.

The spirometric parameter Vital Capacity (VC) is the maximum amount of air that can expel from the lungs after maximal inhalation, and is equal to the sum of inspiratory reserve volume, tidal volume, and expiratory reserve volume.

The spirometric parameter Slow Vital Capacity (SVC) is the amount of air that is inhaled as deeply as possible and then exhaled completely, which measures how deeply a person can breathe.

The spirometric parameter Forced Vital Capacity (FVC) is the volume of air measured in liters, which can forcibly be blown out after full inspiration, and constitutes the most basic maneuver in spirometry tests.

The spirometric parameter Forced Expiratory Volume in the 1st second (FEV₁) is the volume of air that can forcibly be blown out in one second, after full inspiration. Average values for FEV₁ depend mainly on sex and age, whereas values falling between 80% and 120% of the average value are considered normal. Predicted normal values for FEV₁ can be calculated on-site and depend on age, sex, height, weight and ethnicity as well as the research study that they are based on.

The spirometric parameter FEV₁/FVC ratio (FEV₁%) is the ratio of FEV₁ to FVC, which in adults should be approximately 75-80%. The predicted FEV1% is defined as FEV₁% of the patient divided by the average FEV₁% in the appropriate population for that person.

The spirometric parameter Forced Expiratory Flow (FEF) is the flow (or speed) of air coming out of the lung during the middle portion of a forced expiration. It can be given at discrete times, generally defined by what fraction remains of the forced vital capacity (FVC), namely 25% of FVC (FEF₂₅), 50% of FVC (FEF₅₀) or 75% of FVC (FEF₇₅). It can also be given as a mean of the flow during an interval, also generally delimited by when specific fractions remain of FVC, usually 25-75% (FEF₂₅₋₇₅%). Measured values ranging from 50-60% up to 130% of the average are considered normal, while predicted normal values for FEF can be calculated on-site and depend on age, sex, height, weight and ethnicity as well as the research study that they are based on. Recent research suggests that FEF₂₅₋₇₅% or FEF₂₅₋₅₀% may be a more sensitive parameter than FEV₁ in the detection of obstructive small airway disease. However, in the absence of concomitant changes in the standard markers, discrepancies in mid-range expiratory flow may not be specific enough to be useful, and current practice guidelines recommend continuing to use FEV₁, VC, and FEV₁/VC as indicators of obstructive disease.

The spirometric parameter Negative Inspiratory Force (NIF) is the greatest force that the chest muscles can exert to take in a breath, wherein values indicate the state of the breathing muscles.

The spirometric parameter MMEF or MEF refers to maximal (mid-)expiratory flow and is the peak of expiratory flow as taken from the flow-volume curve and measured in liters per second. MMEF is related to peak expiratory flow (PEF), which is generally measured by a peak flow meter and given in liters per minute.

The spirometric parameter Peak Expiratory Flow (PEF) refers to the maximal flow (or speed) achieved during the maximally forced expiration initiated at full inspiration, measured in liters per minute.

The spirometric parameter diffusing capacity of carbon monoxide (D_{L}CO) refers to the carbon monoxide uptake from a single inspiration in a standard time (usually 10 sec). On-site calculators are available to correct D_{L}CO for hemoglobin levels, anemia, pulmonary hemorrhage and altitude and/or atmospheric pressure where the measurement was taken.

The spirometric parameter Maximum Voluntary Ventilation (MVV) is a measure of the maximum amount of air that can be inhaled and exhaled within one minute. Typically this parameter is determined over a 15 second time period before being extrapolated to a value for one minute expressed as liters/minute. Average values for males and females are 140-180 and 80-120 liters per minute respectively.

The spirometric parameter static lung compliance (Cst) refers to the change in lung volume for any given applied pressure. Static lung compliance is perhaps the most sensitive parameter for the detection of abnormal pulmonary mechanics. Cst is considered normal if it is 60% to 140% of the average value of a commensurable population.

The spirometric parameter Forced Expiratory Time (FET) measures the length of the expiration in seconds.

The spirometric parameter Slow Vital Capacity (SVC) is the maximum volume of air that can be exhaled slowly after slow maximum inhalation.

Static intrinsic positive end-expiratory pressure (static PEEPi) is measured as a plateau airway opening pressure during airway occlusion.

The spirometric parameter Maximum Inspiratory Pressure (MIP) is the value representing the highest level of negative pressure a person can generate on their own during an inhalation, which is expresented by centimeters of water pressure (cmH₂O) and measured with a manometer and serves as n indicator of diaphragm strength and an independent diagnostic parameter.

The term “capnography” refers to a technology for monitoring the concentration or partial pressure of carbon dioxide (CO₂) in the respiratory gases. End-tidal CO₂, or ETCO₂, is the parameter that can be determined by capnography.

Gas detection technology is integrated into many medical and other industrial devices and allows the quantitative determination of the chemical composition of a gaseous sample which flows or otherwise captured therein. In the context of embodiments of the present invention, such chemical determination of gases is part of the on-site, noninvasive battery of tests, controlled and monitored activity of the methods presented herein. Gas detectors, as well as gas mixers and regulators, are used to determine and control parameters such as fraction of inspired oxygen level (FiO₂) and the concentration of nitric oxide in the inhaled gas mixture.

According to some embodiments of the present invention, the measurement of vital signs, such as heart rate, blood pressure, respiratory rate and a body temperature, is regarded as part of a battery of on-site and noninvasive measurements.

The phrase “integrated pulmonary index”, or IPI, refers to a patient's pulmonary index which uses information on inhaled/exhaled gases from capnography and on gases dissolved in the blood from pulse oximetry to provide a single value that describes the patient's respiratory status. IPI, which is obtained by on-site and noninvasive techniques, integrates four major physiological parameters provided by a patient monitor (end-tidal CO₂ and respiratory rate as measured by capnography, and pulse rate and blood oxygenation SpO₂ as measured by pulse oximetry), using this information along with an algorithm to produce the IPI score. IPI provides a simple indication in real time (on-site) of the patient's overall ventilatory status as an integer (score) ranging from 1 to 10. IPI score does not replace current patient respiratory parameters, but used to assess the patient's respiratory status quickly so as to determine the need for additional clinical assessment or intervention.

According to some of the embodiments described herein, the monitored physiological or chemical parameters include one or more of the following parameters:

Perfusion Index (PI);

Respiration Rate (RRa);

Oxyhemoglobin Saturation (SpO₂);

Total Hemoglobin (SpHb);

Carboxyhemoglobin (SpCO);

Methemoglobin (SpMet);

Oxygen Content (SpOC); and

Pleth Variability Index (PVI),

and/or at least one off-site parameter selected from the group consisting of: serum nitrite/nitrate (NO₂⁻/NO₃⁻);

serum or urine nitrite/nitrate (NO₂⁻/NO₃⁻) and blood methemoglobin.

According to some of the embodiments described herein, the monitored physiological or chemical parameters include one or more of the following parameters:
Perfusion Index (PI);
Respiration Rate (RRa);
Oxyhemoglobin Saturation (SpO₂);
Total Hemoglobin (SpHb);
Carboxyhemoglobin (SpCO);
Methemoglobin (SpMet);
Oxygen Content (SpOC); and
Pleth Variability Index (PVI),
and/or at least one off-site parameter selected from the group consisting of: serum nitrite/nitrate (NO₂⁻/NO₃⁻); and
skin salinity.

According to some of the embodiments described herein, the method is conducted while monitoring at least one of the following on-site parameters in the gas mixture inhaled by the human subject:

End Tidal CO₂ (ETCO₂);

Nitrogen dioxide (NO₂),

Nitric oxide (NO); and

Fraction of inspired oxygen (FiO₂).

According to some of the embodiments described herein, the monitored physiological or chemical parameters further include one or more of the following parameters:

a urine level of nitrogen dioxide (urine nitrite level) (an off-line parameter);

a vital sign selected from the group consisting of a heart rate, a blood pressure, a respiratory rate and a body temperature (an on-line parameter);

a hematological marker (an off-line parameter), such as, but not limited to, a hemoglobin level, a hematocrit ratio, a red blood cell count, a white blood cell count, a white blood cell differential and a platelet count;

a coagulation parameter (an off-line parameter) such as, but not limited to, a prothrombin time (PT), a prothrombin ratio (PR) and an international normalized ratio (INR);

a serum creatinine level (an off-line parameter);

a liver function marker (an off-line parameter) selected from the group consisting of a aspartate aminotransferase (AST) level, a serum glutamic oxaloacetic transaminase (SGOT) level, an alkaline phosphatase level, and a gamma-glutamyl transferase (GGT) level; a vascular endothelial activation factor (an off-line parameter) selected from the group consisting of Ang-1, Ang-2 and Ang-2/Ang-1 ratio.

It is noted that a sharp elevation of inflammatory biomarkers may be associated with a phenomenon called “cytokine storm”, which has been observed in subjects undergoing nitric oxide inhalation treatment. Hence, monitoring inflammatory biomarkers while performing the method as described herein has an additional role in safety considerations pertaining to the method, according to embodiments of the present invention, wherein no significant increase in inflammatory markers is an indication of safety.

Selected Safety and Efficacy Parameters and Criteria for Monitoring Safety:

According to some embodiments of the present invention, the method as disclosed herein is such that no substantial change is observed in at least one of the monitored physiological parameters or a level of biomarkers pertaining to the safety and efficacy of the treatment presented hereinabove.

In the context of the present embodiments, a change in a parameter or a level of a biomarker is considered substantial when a value of an observation (measurement, test result, reading, calculated result and the likes) or a group of observations falls notably away from a normal level, for example falls about twice the upper limit of a normal level.

A “normal” level of a parameter or a level of a biomarker is referred to herein as baseline values or simply “baseline”. In the context of the present embodiments, the term “baseline” is defined as a range of values which have been determined statistically from a large number of observations and/or measurements which have been collected over years of medical practice with respect to the general human population, a specific sub-set thereof (cohort) or in some cases with respect to a specific person. A baseline is a parameter/biomarker-specific value which is generally and medically accepted in the art as normal for a subject under certain physical conditions. These baseline or “normal” values, and means of determining these normal values, are known in the art. Alternatively, a baseline value may be determined from or in a specific subject before effecting the method described herein using well known and accepted methods, procedures and technical means. A baseline is therefore associated with a range of tolerated values, or tolerance, which have been determined in conjunction with the measurement of a parameter/biomarker. In other words, a baseline is a range of acceptable values which limit the range of observations which are considered as “normal”. The width of the baseline, or the difference between the upper and lower limits thereof are referred to as the “baseline range”, the difference from the center of the range is referred to herein as the “acceptable deviation unit” or ADU. For example, a baseline of 4-to-8 has a baseline range of 4 and an acceptable deviation unit of 2.

In the context of the present embodiments, a significant change in an observation pertaining to a given parameter/biomarker is one that falls more than 2 acceptable deviation unit (2 ADU) from a predetermined acceptable baseline. For example, an observation of 10, pertaining to a baseline of 4-to-8 (characterized by a baseline range of 4, and an acceptable deviation unit of 2), falls one acceptable deviation unit, or 1 AUD from baseline. Alternatively, a change is regarded substantial when it is more than 1.5 ADU, more than 1 ADU or more than 0.5 ADU.

In the context of the present embodiments, a “statistically significant observation” or a “statistically significant deviation from a baseline” is such that it is unlikely to have occurred as a result of a random factor, error or chance.

It is noted that in some parameters/biomarkers or groups of parameters/biomarkers, the significance of a change thereof may be context-dependent, biological system-dependent, medical case-dependent, human subject-dependent, and even measuring machinery-dependent, namely a particular parameter/biomarker may require or dictate stricter or looser criteria to determine if a reading thereof should be regarded as significant. It is noted herein that in specific cases some parameters/biomarkers may not be measurable due to patient condition, age or other reasons. In such cases the method is effected while monitoring the other parameters/biomarkers.

A deviation from a baseline is therefore defined as a statistically significant change in the value of the parameter/biomarker as measured during and/or following a full term or a part term of administration the regimen described herein, compared to the corresponding baseline of the parameter/biomarker. It is noted herein that observations of some parameters/biomarkers may fluctuate for several reasons, and a determination of a significant change therein should take such events into consideration and correct the appropriate baseline accordingly.

Monitoring methemoglobin and serum nitrite levels has been accepted in the art as a required for monitoring the safety of nitric oxide inhalation in a subject. Yet, to date, no clear indication that methemoglobin and serum nitrite levels remain substantially unchanged upon nitric oxide inhalation by a human subject.

According to some embodiments of the present invention, the method comprises monitoring and/or improving at least one of the parameters/biomarkers described hereinabove.

According to some embodiments, the monitored parameter is methemoglobin level.

As methemoglobin levels can be measured using noninvasive measures, the parameter of percent saturation at the periphery of methemoglobin (SpMet) is used to monitor the stability, safety and effectiveness of the method presented herein. Hence, according to some embodiments of the present invention, the followed parameter is SpMet and during and following the administration, the SpMet level does not exceed 5%, and preferably does not exceed 1%. As demonstrated in the Examples section that follows, a SpMet level of subjects undergoing the method described herein does not exceed 1%.

According to some embodiments, the monitored parameter is serum nitrate/nitrite level.

High nitrite and nitrate levels in a subject's serum are associated with nitric oxide toxicity and therefore serum nitrite/nitrate levels are used to detect adverse effects of the method presented herein. According to some embodiments of the present invention, the tested parameter is serum nitrite/nitrate, which is monitored during and following the treatment and the acceptable level of serum nitrite is less than 2.5 micromole/liter and serum nitrate is less than 25 micromole/liter.

According to some of the embodiments described herein, the method is effected while monitoring at least one, at least two, or all on-site parameters which include perfusion index (PI), respiration rate (RRa), oxyhemoglobin saturation (SpO₂/SaO₂/DO), total hemoglobin (SpHb), carboxyhemoglobin (SpCO), methemoglobin (SpMet), oxygen content (SpOC), and pleth variability index (PVI), and/or monitoring at least one or all off-site parameters which include serum nitrite/nitrate level.

According to some of the embodiments described herein, the method is effected while monitoring at least one, at least two, or all on-site parameters in the gas mixture inhaled by the subject, which include end tidal CO₂ (ETCO₂), nitrogen dioxide (NO₂), nitric oxide (NO) and fraction of inspired oxygen (FiO₂).

According to some of the embodiments described herein, the method is effected while monitoring at least one, at least two, or all on-site and/or off-site safety parameters pertaining to nitric oxide inhalation, e.g., methemoglobin formation, and while monitoring at least one, at least two, or all on-site and/or off-site efficacy parameters.

According to some of the embodiments described herein, the method is effected while monitoring at least one, at least two, or all on-site and/or off-site safety parameters pertaining to nitric oxide inhalation, e.g., methemoglobin formation, and while monitoring at least one, at least two, or all on-site and/or off-site efficacy parameters pertaining to CF symptoms, which include, pulmonary functions and/or inflammatory biomarkers.

According to some of the embodiments described herein, the method is effected while monitoring at least one, at least two, or all on-site and/or off-site safety parameters pertaining to nitric oxide inhalation, e.g., methemoglobin formation, and while monitoring at least one, at least two, or all on-site and/or off-site efficacy parameters pertaining to bronchiolitis symptoms, which include, pulmonary functions and/or inflammatory biomarkers.

According to some of the embodiments described herein, the method is effected while monitoring at least one, at least two, or all on-site pulmonary function parameters (spirometric parameters), such as forced expiratory volume (FEV), maximum mid-expiratory flow (MMEF), diffusing capacity of the lung for carbon monoxide (D_{L}CO), forced vital capacity (FVC), total lung capacity (TLC) and residual volume (RV).

For example, the method according to some embodiments is effected while monitoring SpMet as an on-site parameter. Alternatively, the method is effected while monitoring SpMet and ETCO₂ as on-site parameters. Alternatively, the method is effected while monitoring SpMet, ETCO₂ and SpO₂ as on-site parameters.

Alternatively, the method according to some embodiments is effected while monitoring SpMet as one on-site parameter, and one off-site parameter, such as plasma or urine levels of NO₂⁻/NO₃⁻. Alternatively, the method is effected while monitoring SpMet and SpO₂ as on-site parameters, and serum nitrite/nitrate level as one off-site parameter. Alternatively, the method is effected while monitoring SpMet as one on-site parameter, and inflammatory biomarkers in the plasma (for efficacy) and serum nitrite/nitrate level as off-site parameters. Alternatively, the method is effected while monitoring SpO₂ as one on-site parameter, and bacterial load and serum nitrite/nitrate level as off-site parameters. Alternatively, the method is effected while monitoring SpO₂ as one on-site parameter, and inflammatory biomarkers in the plasma and pulmonary function parameters such as FEV₁.

Further alternatively, the method is effected while monitoring SpMet, FEV₁ and SpO₂ as on-site parameters, and inflammatory biomarkers in the plasma and serum nitrite/nitrate level as off-site parameters.

According to some of the embodiments described herein, the method is effected while monitoring at least one, at least two, or all on-site parameters which include SpMet, SpO₂ and FEV₁, and/or monitoring at least one or all off-site parameters which include serum nitrite/nitrate level and inflammatory biomarkers in the plasma, and further monitoring one or more and in any combination of:

a urine NO₂ level (an off-site parameter);

a vital sign (an on-site parameter);

a pulmonary function (an on-site parameter);

a hematological marker (an off-site parameter);

a coagulation parameter (an off-site parameter);

a serum creatinine level (an off-site parameter);

a renal function marker (an off-site parameter);

a liver function marker (an off-site parameter);

a vascular endothelial activation factor (an off-site parameter).

According to some of the embodiments described herein, the method is effected while monitoring at least one, at least two, or all on-site chemical parameters in the inhaled gas mixture, such as FiO₂ and NO₂.

It is noted herein that for any of the abovementioned embodiments, that the method is effected while no substantial change is observed in any one or more than one or all of the monitored parameters described herein.

According to some embodiments of the present invention, the method is effected while monitoring urine nitrite levels, such that the urine nitrite level is substantially unchanged during and subsequent to carrying out the method as presented herein. It is noted herein that urine nitrite levels may fluctuate for several known reasons, and a determination of a significant change therein should take such events into consideration and correct the appropriate baseline accordingly.

According to some embodiments of the present invention, hematological markers, such as the hemoglobin level, the hematocrit ratio, the red blood cell count, the white blood cell count, the white blood cell differential and the platelet count, are substantially unchanged during and subsequent to carrying out the method as presented herein.

According to some embodiments of the present invention, vascular endothelial activation factors, such as Ang-1, Ang-2 and Ang-2/Ang-1 ratio, as well as the serum creatinine level and various liver function markers, such as the aspartate aminotransferase (AST) level, the serum glutamic oxaloacetic transaminase (SGOT) level, the alkaline phosphatase level, and the gamma-glutamyl transferase (GGT) level, are substantially unchanged during and subsequent to carrying out the method as presented herein.

Oxygenation of the subject can be assessed by measuring the subject's saturation of peripheral oxygen (SpO₂). This parameter is an estimation of the oxygen saturation level, and it is typically measured using noninvasive measures, such as a pulse oximeter device. Hence, according to some embodiments of the present invention, the followed parameter during and following the administration is SpO₂, and the level of SpO₂ is higher than about 89%.

According to some embodiments of the present invention, various vital signs, such as the heart rate, the blood pressure, the respiratory rate and the body temperature; and various coagulation parameters, such as the prothrombin time (PT), the prothrombin ratio (PR) and the international normalized ratio (INR), are substantially unchanged during and subsequent to carrying out the method as presented herein. It is noted that these parameters are regarded as an indication that the general health of the subject is not deteriorating as a result of the medical condition and/or the treatment.

According to some embodiments, the aforementioned general health indicators show an improvement during and subsequent to carrying out the method as presented herein, indicating that the treatment is beneficial to the subject.

Thus, according to some embodiments of the present invention, the method as disclosed herein is effected such that general health indicators as described herein are at least remained unchanged or are improved.

Modes of Administration and Inhalation Devices:

The human subject can be subjected to the inhalation by active or passive means.

By “active means” it is meant that the gaseous mixture is administered or delivered to the respiratory tract of the human subject. This can effected, for example, by means of an inhalation device having a delivery interface adapted for human respiratory organs. For example, the delivery interface can be placed intermittently on the human subject's respiratory organs, whereby when it is removed, the subject breaths ambient air or any other gaseous mixture that is devoid of nitric oxide, as defined herein.

By “passive means” it is meant that the human subject inhales a gaseous mixture containing the indicated dose of nitric oxide without devices for delivering the gaseous mixture to the respiratory tract.

For example, the subject can be subjected to 160 ppm or more nitric oxide in an intermittent regimen by entering and exiting an atmospherically controlled enclosure filled with the nitric oxide-containing mixture of gases discussed herein, or by filling and evacuating an atmospherically controlled enclosure which is in contact with a subject's respiratory tract.

According to some embodiments of the present invention, in any of the methods of treatment presented herein, the nitric oxide administration can be effected by an inhalation device which includes, without limitation, a stationary inhalation device, a portable inhaler, a metered-dose inhaler and an intubated inhaler.

An inhaler, according to some embodiments of the present invention, can generate spirometry data and adjust the treatment accordingly over time as provided, for example, in U.S. Pat. No. 5,724,986 and WO 2005/046426. The inhaler can modulate the subject's inhalation waveform to target specific lung sites. According to some embodiments of the present invention, a portable inhaler can deliver both rescue and maintenance doses of nitric oxide at subject's selection or automatically according to a specified regimen.

According to some embodiments of the present invention, an exemplary inhalation device may include a delivery interface adaptable for inhalation by a human subject.

According to some embodiments of the present invention, the delivery interface includes a mask or a mouthpiece for delivery of the mixture of gases containing nitric oxide to a respiratory organ of the subject.

According to some embodiments of the present invention, the inhalation device further includes a nitric oxide analyzer positioned in proximity to the delivery interface for measuring the concentration of nitric oxide, oxygen and nitrogen dioxide flowing to the delivery interface, wherein the analyzer is in communication with the controller.

According to some embodiments of the present invention, subjecting the subject to the method described herein is carried out by use of an inhalation device which can be any device which can deliver the mixture of gases containing nitric oxide to a respiratory organ of the subject. An inhalation device, according to some embodiments of the present invention, includes, without limitation, a stationary inhalation device comprising tanks, gauges, tubing, a mask, controllers, values and the likes; a portable inhaler (inclusive of the aforementioned components), a metered-dose inhaler, a an atmospherically controlled enclosure, a respiration machine/system and an intubated inhalation/respiration machine/system. An atmospherically controlled enclosure includes, without limitation, a head enclosure (bubble), a full body enclosure or a room, wherein the atmosphere filling the enclosure can be controlled by flow, by a continuous or intermittent content exchange or any other form of controlling the gaseous mixture content thereof.

According to some embodiments of the invention, the intermittent inhalation is effected by intermittently subjecting the human subject to a gaseous mixture (the inhalant) by breathing cycle-coordinated pulse delivery, which contains nitric oxide at the indicated concentration (a nitric oxide-containing gaseous mixture). This mode of inhalation is referred to herein as intermittent breathing cycle-coordinated pulse delivery inhalation.

According to an alternative aspect of some embodiments of the present invention, there is provided a method of treating an inflammatory disease or disorder in a human subject, which includes subjecting the human subject to intermittent inhalation of an inhalant, whereas the intermittent inhalation includes at least one cycle of a breathing cycle-coordinated pulse delivery inhalation of the inhalant for a first time period, followed by inhalation of essentially no nitric oxide for a second time period, wherein the breathing cycle-coordinated pulse delivery inhalation is configured to deliver about 80 ppm-hour of nitric oxide during at least one cycle.

In the context of embodiments of the present invention, the term “nitric oxide-load” (“NO-load”) refers to a certain cumulative amount of nitric oxide to which a subject, or a pathogen, is exposed to during inhalation treatment (e.g., the presently claimed treatment), which is estimated in terms of ppm-hour, namely the average concentration of nitric oxide in the inhalant multiplied by the overall time of exposure. The nitric oxide-load can be estimated per cycle of the treatment (NO-load per cycle), or per a time unit, such as a day (daily NO-load).

According to some embodiments of the present invention, the intermittent delivery of nitric oxide to the subject is conducted such that the subject inhales nitric oxide at an nitric oxide-load that ranges from 600 ppm-hour to 2000 ppm-hour daily, wherein the intermittent delivery is effected such that the daily nitric oxide-load is inhaled in more than one session of uninterrupted administration.

According to some embodiments of the present invention, the intermittent delivery is effected such that the daily nitric oxide-load is inhaled in one or more sessions of intermittent breathing cycle-coordinated pulse delivery inhalation, while the nitric oxide-load per cycle of each cycle is at least about 80 ppm-hour. Such nitric oxide-load per cycle can be obtained, for example, by configuring the pulse(s) to deliver, during one cycle, an inhalant having 160 ppm of nitric oxide for 30 minutes (the first time period). It is noted that other concentrations and other first time periods, which afford a nitric oxide-load of at least 80 ppm-hour per cycle, are also contemplated and encompassed by embodiment of the present invention.

By “intermittent breathing cycle-coordinated pulse delivery inhalation” it is meant that the subject is subjected to a gaseous mixture that contains the indicated concentration of nitric oxide intermittently, and thus inhales such a nitric oxide-containing gaseous mixture by breathing cycle-coordinated pulse delivery two or more times with intervals between each inhalation. The subject therefore inhales the nitric oxide-containing gaseous mixture, then stops inhaling a nitric oxide-containing gaseous mixture by breathing cycle-coordinated pulse delivery and inhales instead a gaseous mixture that does not contain the indicated concentration of nitric oxide (e.g., air), then inhales again the nitric oxide-containing gaseous mixture by breathing cycle-coordinated pulse delivery, and so on and so forth.

In some embodiments of this aspect of the present invention, “a nitric oxide-containing gaseous mixture” is used to describe a gaseous mixture that contains at least 160 ppm nitric oxide. The nitric oxide-containing mixture can comprise 160 ppm, 170 ppm, 180 ppm, 190 ppm, 200 ppm and even higher concentrations of nitric oxide. Other gaseous mixtures mentioned herein include less than 160 ppm nitric oxide or are being essentially devoid of nitric oxide, as defined herein.

In some embodiments “a nitric oxide-containing gaseous mixture” describes a gaseous mixture that delivers nitric oxide at 80 ppm-hour.

By “essentially devoid of nitric oxide” it is meant no more than 50 ppm, no more than 40 ppm, no more than 30 ppm, no more than 20 ppm, no more than 10 ppm, no more than 5 ppm, no more than 1 ppm and no more than ppb, including absolutely no nitric oxide.

According to some embodiments of the present invention, the intermittent breathing cycle-coordinated pulse delivery inhalation includes one or more cycles, each cycle comprising breathing cycle-coordinated pulse delivery inhalation of a gaseous mixture containing nitric oxide at the specified concentration (e.g., at least 160 ppm) for a first time period, which is also referred to herein as the nitric oxide-load per cycle, followed by inhalation of a gaseous mixture containing no nitric oxide for a second time period. According to some embodiments of the present invention, during the second period of time the subject may inhale ambient air or a controlled mixture of gases which is essentially devoid of nitric oxide, as defined herein.

In some embodiments, the first time period spans from 10 to 45 minutes, or from 20 to 45 minutes, or from 20 to 40 minutes, and according to some embodiments, spans about 30 minutes.

According to some embodiments of the present invention, the second time period ranges from 3 to 5 hours, or from 3 to 4 hours, and according to some embodiments the second time period spans about 3.5 hours.

According to some embodiments of the present invention, this inhalation regimen is repeated 1-6 times over 24 hours, depending on the duration of the first and second time periods.

In some embodiments, a cycle of intermittent breathing cycle-coordinated pulse delivery of nitric oxide, e.g., 160 ppm for 30 minutes followed by 3.5 hours of breathing no nitric oxide, is repeated from 1 to 6 times a day. According to some embodiments, the cycles are repeated 5 times a day.

In some embodiments, a cycle of intermittent breathing cycle-coordinated pulse delivery of nitric oxide, e.g., at nitric oxide-load of 80 ppm-hour per cycle, followed by 3.5 hours of breathing no nitric oxide, is repeated from 1 to 6 times a day. According to some embodiments, the cycles are repeated 5 times a day.

According to some embodiments of the present invention, the regimen of 1-5 cycles of intermittent breathing cycle-coordinated pulse delivery of nitric oxide per day is carried out for 1 to 7 days, or from 2 to 7 days, or from 3 to 7 days, or for 1, 2, 3, 4 or 5 successive weeks. According to some embodiments of the present invention, the intermittent breathing cycle-coordinated pulse delivery inhalation is effected during a time period of 14 days. However, longer time periods of intermittent nitric oxide administration as described herein, are also contemplated.

According to embodiments of the present invention, the nitric oxide-containing gaseous mixture, which the subject inhales during the first time period, is generated in-situ in an inhalation device which is configured to respond to the subject's breathing cycle such that nitric oxide is mixed into the inhalant in one or more pulses when the subject breaths in at a high rate, namely at the inhalation period of the breathing cycle. This mode of administration of nitric oxide by inhalation is referred to herein as “breathing cycle-coordinated pulse delivery inhalation”.

In the context of embodiments of the present invention, the term “pulse” refers to a mode of administering nitric oxide, which is introduced into the inhalant in interrupted and concentrated doses during a predetermined period of time, referred to herein as the “pulse delivery period”, wherein each pulse, effected during the pulse delivery period, spans a predetermined period of time, referred to herein as the “pulse-on period”, and interrupted by a “pulse-off period”.

According to embodiments of the present invention, the pulse delivery period starts during the inhalation period, after a period of time which is referred to herein as the “pulse delay period”. According to some embodiments of the present invention, the pulse delivery period is typically shorter than the inhalation period, and the time between the end of the pulse delivery period and the end of the inhalation period is referred to herein as the “pulse cessation period”.

According to some embodiments of the present invention, the inhalation device for delivering the breathing cycle-coordinated pulse delivery inhalation of gashouse nitric oxide is configured to detect the various phases of the breathing cycle, namely the onset of the inhalation and the exhalation periods, and can therefore coordinate the pulses with the breathing cycle such that the pulse delay period is coordinated to start as soon as the rate of intake increases at the onset of the inhalation period, and the pulse cessation period is coordinated to start with as soon as the rate of intake decreases close to the end of the inhalation period.

In some embodiments, the length of the various time periods in the breathing cycle-coordinated pulse delivery inhalation scheme is determined and/or calculated relative to the duration of the breathing cycle, namely in percent of the total duration of the breathing cycle, or parts thereof. For example, the duration of the inhalation period is determined by sensing the flow rate of the inhalant, and the pulse delay period is automatically set to 20% of the inhalation period. Consequently, the pulse delivery period can be set to 60% of the inhalation period, and the pulse cessation period is the remaining 20% of the inhalation period. The number of pulses, namely the pulse-on and pulse-off periods, can be set similarly according to the duration of the pulse delivery period. For example, the number of pulses can be set to one, namely a pulse that spans the entire duration of the pulse delivery period. This example may be suitable for a subject experiencing shortness of breath or any difficulty in respiration. Alternatively, in cases where the subject is breathing normally, the pulse-on period is set to 200-300 milliseconds (ms), and the pulse-off period is set to 100 ms, while the number of pulses is automatically set by the duration of pulse delivery period which is derived from the measured inhalation period.

In some embodiments, the pulse delay period ranges from 0 ms to 2500 ms. Alternatively, in some embodiments, the pulse delay period ranges from 0% to 80% of the inhalation period.

In some embodiments, the pulse cessation period ranges from 0 ms to 2500 ms. Alternatively, in some embodiments, the pulse cessation period ranges from 80% to 0% of the inhalation period.

In some embodiments, each the pulse-on periods individually ranges from 100 ms to 5000 ms. Alternatively, each the pulse-on periods individually ranges from 10% to 100% of the inhalation period.

In some embodiments, each the pulse-off period individually ranges from 0 ms to 2500 ms. Alternatively, each the pulse-off periods individually ranges from 0% to 200% of the pulse-on period.

In some embodiments, the method is based on a single pulse per inhalation period. In some embodiments, the single pulse is effected such that the pulse delivery period starts essentially as the inhalation period starts (pulse delay period is essentially zero), and ends essentially as the inhalation period ends (pulse cessation period is essentially zero). In other embodiments the method is effected by using a single pulse that starts after the inhalation period starts, and ends before the inhalation ends.

In some embodiments, the coordination of pulse delivery is set to deliver more than one pulse in succession during the pulse delivery period, until the device senses a decrease in the rate of intake close to the end of the inhalation period. In such embodiments, the device is set to interrupt each pulse-on period with a pulse-off period. In some embodiments, the device is set to deliver a predetermined number of pulses that ranges from 1 to 2, from 1 to 3, from 1 to 4, from 1 to 5, from 1 to 6, from 1 to 7, from 1 to 8, from 1 to 9, from 1 to 10, or from 1 to any number of pulses that can take place within the pulse delivery period as determined by any given breathing cycle. It is further noted that each of the pulses may span a different pulse-on period and be interrupted by a pulse-off period of different lengths.

The concentration of nitric oxide in the nitric oxide-containing gaseous mixture is controlled by the concentration of nitric oxide is introduced into the inhalant, the output by which nitric oxide is introduced into the inhalant, the duration of the pulse-on period and the number of pulses introduced into the inhalant during the pulse delivery period. According to some embodiments of the present invention, during the pulse delivery period the inhalant is essentially a nitric oxide-containing gaseous mixture which contains at least 160 ppm nitric oxide, or nitric oxide-load of 80 ppm-hour per cycle, while during the pulse delay period and the pulse cessation period the inhalant is essentially devoid of nitric oxide.

According to some embodiments, the method is effected by using more than one pulse, wherein the inhalant, which is produced by each of the pulses, delivers to the patient a different concentration of nitric oxide. For example, the method may be carried out by administering to the patient, during the pulse delivery period, three pulses, such that the inhalant that stems from the first pulse is characterized by an nitric oxide concentration of 160 ppm, the inhalant that stems from the second pulse is characterized by an nitric oxide concentration of 80 ppm, and the inhalant that stems from the first pulse is characterized by an nitric oxide concentration of 100 ppm. Hence, at least one pulse effects a concentration of at least 160 ppm. In other examples, some of the pulses may deliver an inhalant characterized by an nitric oxide concentration of more than 160 ppm.

Alternatively, the number of pulses, the concentration of nitric oxide in each of the pulses, and the duration of the first time period during which pulses are generated, are configured to deliver an nitric oxide-load per cycle of 80 ppm-hour.

As presented hereinabove, breathing cycle-coordinated pulse delivery inhalation allows the introduction of high concentrations of nitric oxide essentially during the periods of time in which the subject inhales at the highest in-breathing rate, thereby minimizing exposure of parts of the respiratory tract to high concentrations of nitric oxide. For example, since nitric oxide is introduced in pulses after the beginning of the inhalation period and before the end of the inhalation period, parts of the upper respiratory tract, the trachea and the some of the respiratory tree in the lungs which are not rich with alveolor capillaries, are only briefly exposed to high concentrations of nitric oxide due to the rate of inhalant intake, while the alveoli are exposed to this high concentrations of nitric oxide for a longer period of time.

According to some embodiments of the present invention, subjecting the subject to the method described herein is carried out by use of an inhalation device which can be any device which can deliver the mixture of gases containing nitric oxide, including but not limited to breathing cycle-coordinated pulse delivery to a respiratory organ of the subject. An inhalation device, according to some embodiments of the present invention, includes, without limitation, a stationary inhalation device comprising tanks, gauges, tubing, a mask, controllers, values and the likes; a portable inhaler (inclusive of the aforementioned components), a metered-dose inhaler, a respiration machine/system and an intubated inhalation/respiration machine/system.

Exemplary inhalation devices which may be suitable for the execution of any embodiment of any of the methods described herein, are provided, for example, by U.S. Provisional Patent Application Nos. 61/876,346 and 61/969,201, and U.S. Pat. Nos. 6,164,276 and 6,109,260, the contents of which are hereby incorporated by reference. Commercial inhalation devices which may be suitable for the execution of any of the methods described herein, include the INOpulse® DS-C developed by Ikaria Australia Pty Ltd, or the Ohmeda INOpulse Delivery System by Datex-Ohmeda.

An inhaler, according to some embodiments of the present invention, can generate spirometry data and adjust the treatment accordingly over time as provided, for example, in U.S. Pat. No. 5,724,986 and WO 2005/046426, the contents of which are hereby incorporated by reference. The inhaler can modulate the subject's inhalation waveform to target specific lung sites. According to some embodiments of the present invention, a portable inhaler can deliver both rescue and maintenance doses of nitric oxide at subject's selection or automatically according to a specified regimen.

According to some embodiments of the present invention, an exemplary inhalation device may include a delivery interface adaptable for inhalation by a human subject. According to some embodiments of the present invention, the delivery interface includes a mask or a mouthpiece for delivery of the mixture of gases containing nitric oxide to a respiratory organ of the subject.

According to some embodiments of the present invention, the inhalation device further includes a nitric oxide analyzer positioned in proximity to the delivery interface for measuring the concentration of nitric oxide, oxygen and nitrogen dioxide flowing to the delivery interface, wherein the analyzer is in communication with the controller.

It is expected that other methods for treating an inflammatory disease or disorder by intermittent inhalation of nitric oxide at 160 ppm or more will be developed and the scope of the term treating an inflammatory disease or disorder by intermittent inhalation of nitric oxide is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub combination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1

### Nitric Oxide Inhalation in Human Subjects Diagnosed with, or Suffering from CF

The present open-label Phase 2 clinical study presented herein examined some aspects of the use of NO as an adjuvant therapy for CF. Chronic microbial lung infections, particularly with P. aeruginosa, are the leading cause of morbidity and mortality in human subjects diagnosed with, or suffering from CF. The aim of the following study is to assess the safety and tolerability of NO inhalations in human subjects diagnosed with, or suffering from CF.

CF Symptoms:

In some human subjects diagnosed with, or suffering from CF, symptoms are observed during infancy, but others with CF may not experience symptoms until adolescence or adulthood. Fifty percent of human subjects diagnosed with CF present with pulmonary manifestations that often begin during infancy. Adolescents may have retarded growth, delayed onset of puberty, and a declining tolerance for exercise.

The thick and sticky mucus caused by CF in the lung can result in respiratory symptoms such as persistent cough, wheezing, repeated lung infections, and repeated sinus infections. Other symptoms include intercostal retractions, use of accessory muscles of respiration, a barrel-chest deformity, digital clubbing, and cyanosis, which occur with disease progression. Pulmonary complications in adolescents and adults include bronchiectasis (widened, scarred airways), sinusitis, chronic infections, airway obstruction, pneumothorax (collapsed lung), and respiratory failure. Indeed, lung diseases accounts for more than 90% of deaths in human subjects diagnosed with, or suffering from CF. The thick mucus caused by CF can also affect the gastrointestinal system preventing digestive enzymes from pancreas from reaching the intestine. The result is that the intestines cannot absorb nutrients well. The symptoms that result include foul-smelling, greasy stools; poor weight gain and growth; constipation; and intestinal blockage.

The above observations lead to the conclusion that lung function and systemic inflammation biomarkers can be used effectively to monitor CF treatment safety as well as efficacy.

CF Diagnostics:

Diagnostic tests for CF [Voter, K. Z. and Ren, C. L., Clin. Rev. Allerg. Immunol., 2008, 35, p. 100-106] include blood tests for a particular components that are commonly elevated in babies with CF, sweat tests to assess salt content (one of the first signs of CF is a salty taste to the skin caused a tendency to have a higher than normal amount of salt in human subjects diagnosed with, or suffering from CF′ sweat), and genetic testing to test for specific mutations on the gene responsible for CF.

The sweat test remains the standard diagnostic test for CF; it measures the amount of salt in a child's sweat, with a high salt level indicating that an individual has CF. For human subjects with indeterminate sweat test results, the following diagnostic studies should be considered to evaluate human subjects: evaluation for respiratory disease (respiratory tract cultures, assessment for bronchiectasis with computed tomography, evaluation of paranasal sinuses); quantitative assessment of pancreatic function by fecal elastase measurement; evaluation of liver function by liver biopsy; and male genital tract evaluation (semen analysis, urologic examination, ultrasonography, and scrotal exploration).

Current CF Therapy:

The currently available therapies for the pulmonary symptoms of CF include airway clearance techniques to loosen and get rid of mucous from the lung, mucolytics to thin mucus so human subjects can cough it out easier, and antibiotics to fight infection-causing bacteria [Döring, G. et al., Journal of Cystic Fibrosis, 2012, 11, p. 461-479]. Among the medicines used to treat CF are antibiotics to treat and prevent lung infections, mucus-thinning drugs that reduce the stickiness of the mucus, and bronchodilators to help keep airways open. Mechanical devices such as a chest clapper or an inflatable vest help to loosen chest mucus. Surgical and other procedures can include a feeding tube to deliver extra nutrition, a lung transplant, or bowel surgery. Table 1 lists current pharmacological interventions and means currently in use to treat the multisystem manifestations of CF.

TABLE 1

| Therapeutic mean | Therapeutic action |
|---|---|
| Inhaled hypertonic saline, 7% | Restores mucus clearance and pul- |
| | monary function; becoming standard |
| | of care for human subjects diagnosed |
| | with, or suffering from CF |
| rDNAse | Increases mucus clearance by de- |
| | grading DNA in sputum |
| Antibiotics such as: | Treat lung infections |
| Azithromycin (250 mg/day or | |
| 500 mg three times a week) | |
| Inhaled aminoglycosides (e.g., | |
| tobramycin 300 mg bid) | |
| Oral semisynthetic penicillin, | |
| cephalosporin + inhaled tobramycin | |
| or colistin (75 mg bid) for mild | |
| acerbations | |
| IV antibiotics for severe | |
| exacerbations | |
| Inhaled β-adrenergic agonists | Control airway constriction |
| Oral glucocorticoids | Reduce airway inflammation |
| | Treatment for allergic broncho- |
| | pulmonary aspergillosis |
| Pancreatic enzyme replacement | Facilitates digestion and absorption |
| (Lipase) | of nutrients |
| Vitamins E, K | Replacement of fat soluble vitamins |
| Insulin | Treatment of hyperglycemia |
| Megalodiatrizoate or other | Treatment of acute distal intestinal |
| hypertonic radiocontrast materials | obstruction |
| via enema | |

Clinical Method:

NO was administered via inhalation to nine clinically stable human subjects diagnosed with, or suffering from CF from two medical centers. Patients received three daily 30-minute treatments of 160 ppm NO with at least 3.5 hours between treatments, for 5 days per week over a two-weeks time period.

Safety parameters, including NO and nitrogen dioxide (NO₂ via NO₂⁻/NO₃⁻) concentrations, inhaled fraction of inspired oxygen (FiO₂), methemoglobin level (SpMet, or “% MetHb”), and oxyhemoglobin (oxygen) saturation (SpO₂ or SaO₂), were continuously monitored using a noninvasive pulse-oximetry device RAD-57™ or RAD-87™ by Masimo Irvine, Calif. These devices also monitor Perfusion Index (PI), Respiration Rate (RRa), Total Hemoglobin (SpHb), Carboxyhemoglobin (SpCO), Oxygen Content (SpOC) and Pleth Variability Index (PVI®). Vital signs, including blood pressure, pulse, and respiratory rate were also closely monitored.

Preliminary efficacy measures included determination of microbial density in sputum and measurements of forced exhaled volume at 1 second (FEV₁). Inflammation associated with C-reactive protein (CRP) levels was assessed as a secondary outcome measure. Other inflammation biomarkers, such as interleukins, were also monitored (data not shown).

Bacterial load was measured for exemplary, non-limiting selection of species that included, P. alcaligenes, methicillin-sensitive Staphylococcus aureus (MSSA), Achromobacter spp., A. fumigates, non-mucoid P. aeruginosa and mucoid P. aeruginosa.

Clinical Results:

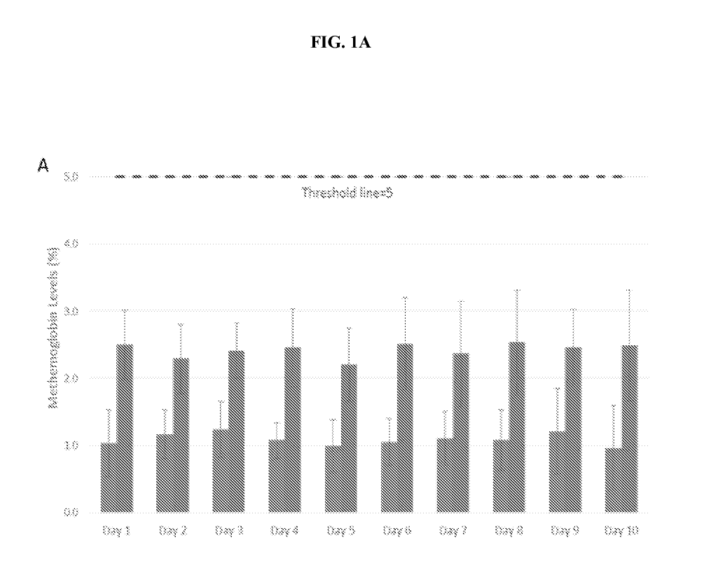

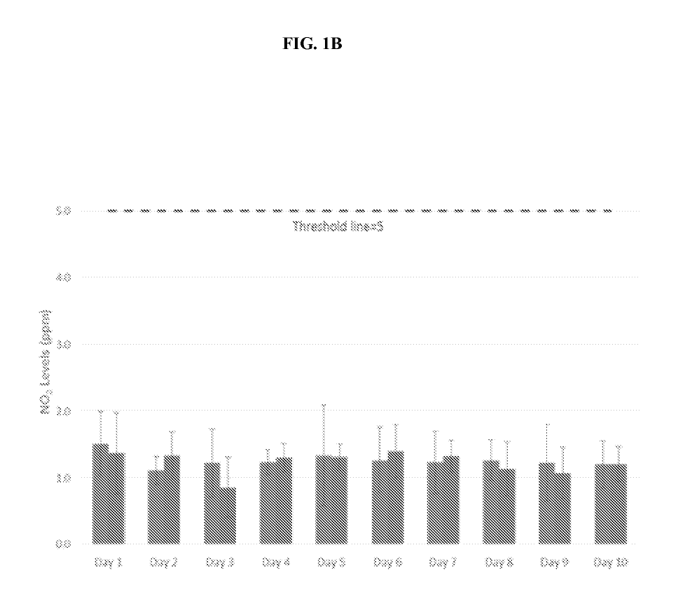

FIGS. 1A-B present comparative bar plots, showing average change in MetHb percent levels (FIG. 1A) and NO₂ levels in ppm (FIG. 1B) prior to first treatment (blue) and after last treatment (red) (threshold value of 5% is shown as a dotted red line), as measured in 9 human subjects during 10 days of treatment, according to some embodiments of the present invention.

As can be seen in FIGS. 1A-B, all human subjects tolerated the treatment and completed the study per protocol. No serious adverse events and no clinically significant changes in vital signs were observed during the study. Oxygen saturation post treatment remained at more than 92% in all of the human subjects. Average MetHb levels post treatment were 2.4%±0.5% (mean±SD) and remained well beneath the safety limit of 5% throughout the trial in all human subjects. Average NO₂ levels were 1.2 ppm±0.4 ppm (mean±SD) post treatment and remained less than 3 ppm in all human subjects.

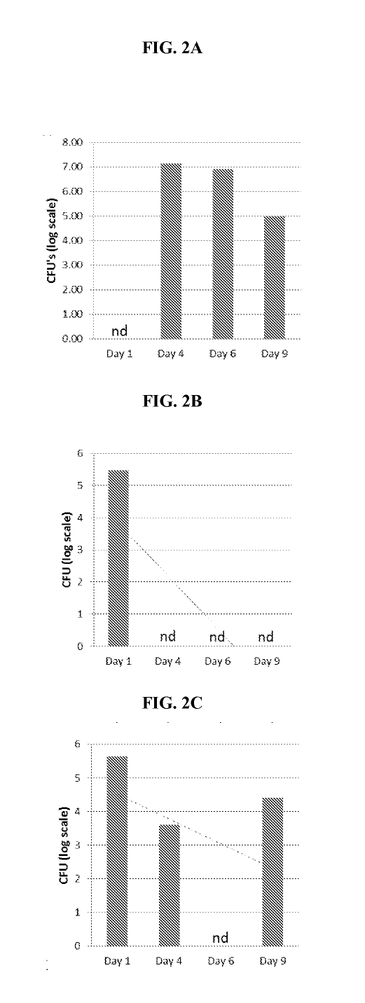

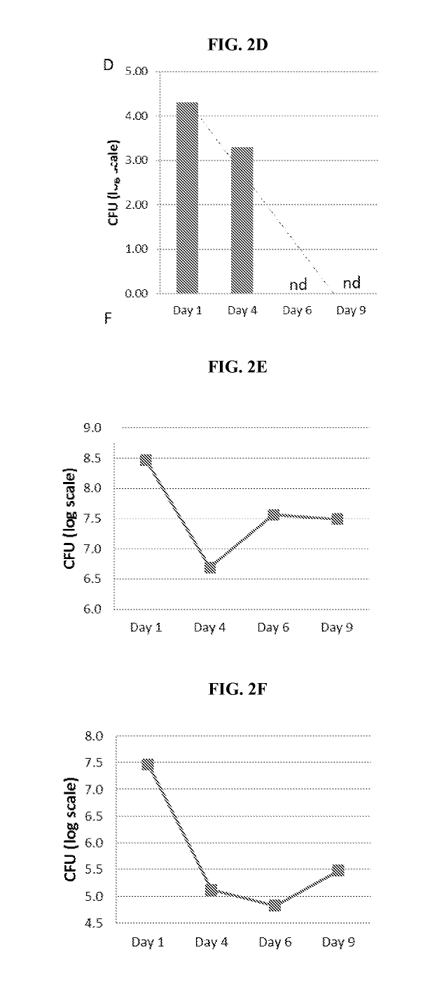

FIGS. 2-F present results of CFU determination of P. alcaligenes in “Patient 1” (CFSCH01) (FIG. 2A), Methicillin-sensitive Staphylococcus aureus (MSSA) in “Patient 3” (CFSCH03) (FIG. 2B), Achromobacter spp. in “Patient 3” (FIG. 2C), A. fumigatus in “Patient 3” (FIG. 2D), non-mucoid P. aeruginosa in “Patient 4” (CFSCH04) (FIG. 2E), and mucoid P. aeruginosa in “Patient 4” (FIG. 2F), throughout the treatment, whereas “nd” stands for non-detected levels.

As can be seen in FIGS. 2-F, three out of nine human subjects had significant log decreases in microbial density measured by colony forming units (CFUs) during treatment. By Day 9, “Patient 1” (CFSCH01) had a 2-log decrease in P. alcaligenes CFUs. “Patient 3” (CFSCH03) had a 1-log decrease in Achromobacter spp., and elimination of methicillin-sensitive S. aureus (MSSA) and A. fumigatus CFU counts. There was 1-log decrease in nonmucoid P. aeruginosa and a 2-log decrease in mucoid P. aeruginosa counts in “Patient 4” (CFSCH04). All of the remaining six human subjects showed either modulation in their bacterial counts (one patient) or no significant change in CFU of all tested microorganisms (data not shown). Notably, CFU determination of microbial counts revealed a 2- to 2.5-log decrease in four of the nine human subjects during the first 4 days of treatment.

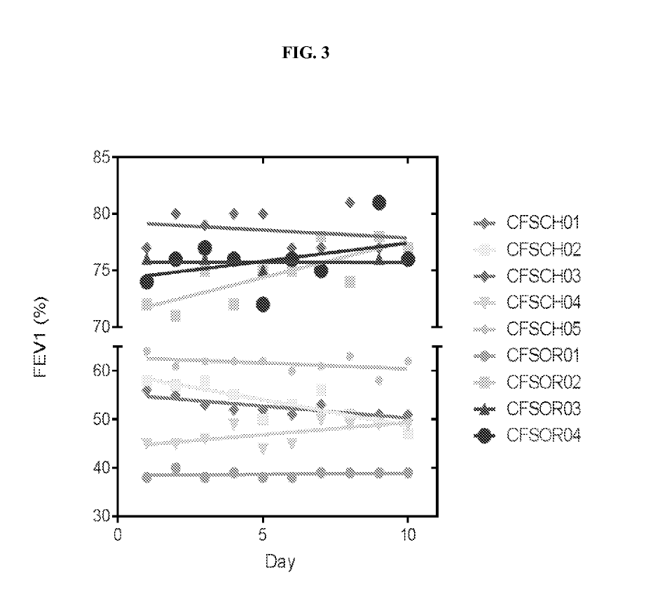

FIG. 3 presents a comparative plot showing the linear trend of FEV₁ measurements as taken from 9 human subjects diagnosed with, or suffering from CF treated with 160 ppm NO three times/day with at least 3.5 hours between treatments for 10 days from screening to end of treatment, according to some embodiments of the present invention.

As can be seen in FIG. 3, FEV₁ values in 4 human subjects increased by 3% to 9%. Two human subjects had 3% to 9% reduction in FEV₁ and one patient had a measured FEV₁ reduced by 11%, which returned to initial levels after 2 weeks. Two human subjects had no significant changes in FEV₁ values.

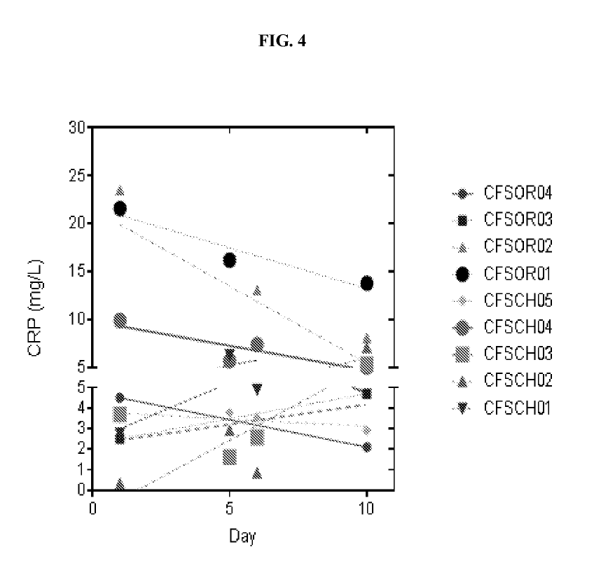

FIG. 4 presents a comparative plot showing the linear trend of CRP levels in mg/L as measured in 9 human subjects diagnosed with, or suffering from CF treated with 160 ppm NO three times/day with at least 3.5 hours between treatments for 10 days from screening to end of treatment, according to some embodiments of the present invention.

As can be seen in FIG. 4, three out of nine human subjects exhibited signs of inflammation at baseline, with CRP levels greater than 5 mg/L. All three human subjects showed decreased inflammation, with 40-60% reduction in CRP levels, after treatment.

In summary, inhalation of 160 ppm NO for 30 min, 3 times daily for 5 consecutive days per week with a break of at least 3.5 hours between inhalations over a 2 week period, is safe and well tolerated in human subjects diagnosed with CF. The treatment resulted in differential yet significant reduction of microbiological load, while reduced inflammatory state was observed after NO treatment in human subjects with active inflammation.

### Example 2

### Inflammation Treatment by Nitric Oxide Inhalation

A cohort of human subjects diagnosed with inflammation are treated by intermittent inhalation of nitric oxide according to the regimen presented in Example 1 hereinabove, namely inhalation of 160 ppm nitric oxide for 30 minutes, 3 times daily for 5 consecutive days per week with a break of at least 3.5 hours between inhalations over a 2 week period.

Inflammatory Biomarkers in Blood/Serum:

Blood/serum levels of inflammatory biomarkers, such as CRP, TNFα, IL-1β, IL-6, IL-8, IL-10 and IL-12p70, are determined before, after and throughout the treatment, using blood withdrawal and analysis procedures.

Human Magnetic Luminex Screening Assay, by R&D Systems, is an exemplary analysis procedure. It is a flexible bead-based multiplex for the Luminex® platform that can allow up to 100 user-defined target analytes to be simultaneously profiled using cell culture supernates, serum, or plasma samples in the polystyrene bead format, and up to 50 analytes can be screened in the magnetic bead format. This platform is suitable for assaying inflammatory biomarkers, such as, but not limited to, CXCL8/IL-8, GM-CSF, ICAM-1, IFN-gamma, IL-1 beta, IL-1ra/IL-1F3, IL-2, IL-4, IL-5, IL-6, IL-10, IL-12 p70, IL-17A, MMP-8, MMP-9, TNF-alpha, TNF RII and VEGF.

Inflammatory Biomarkers in Induced Sputum:

Induced sputum is collected before, after and throughout the treatment and analyzed for inflammatory biomarkers which include neutrophils count, IL-8 level and neutrophil elastase (NE) activity.

A portion of the induced sputum specimen is transferred to a microbiology laboratory for culture. Mucoid portions selected from a petri dish are weighed. Fresh dithiothreitol (DTT), 0.1%, diluted 1:10 with distilled water, is added 2:1 volume/sputum weight, pipetted vigorously, and homogenized in a shaking water bath at 37° C. for 15 minutes; and an equal volume phosphate-buffered saline solution is added to stop the reaction. The cell suspension is filtered through 52 μm nylon gauze and centrifuged. The supernatant is stored at −70° C. The pellet, diluted with a solution of Roswell Park Memorial Institute medium plus 10% fetal calf serum to a concentration of 10/μL, is cytocentrifuged and stained with Giemsa stain. Several hundred nonsquamous cells are counted and the results expressed as a percentage of the total nonsquamous count. Supernatants are analyzed for IL-8 by enzyme-linked immunosorbent assay (ELISA) and for neutrophil elastase activity, using commercially available kits.

For example, in a typical induced sputum assay, an average baseline level of IL-8, neutrophil elastase, eosinophil cationic protein (ECP), eotaxin, tryptase and RANTES levels in sputum in a non-smoking and generally healthy human, is about 1.16 ng·mL⁻¹, 0.36 μg·mL⁻¹, 0.02 μg·mL⁻¹, 12.9 pg·mL⁻¹, less than 2 ng·mL⁻¹ and 35.3 pg·mL⁻¹, respectively. Additional experimental assay and analyses procedures for following inflammatory biomarkers such as neutrophils, lymphocytes, eosinophils, IL-8, neutrophil elastase, eotaxin, tryptase and RANTES, according to some embodiments of the present invention, can be found, for example, in Fujimoto, K. et al. [Eur Respir J., 2005, 25(4), pp. 640-6].

Luminex® platform may also be used to assay any inflammatory biomarkers in induced sputum samples, according to some embodiments of the present invention.

### Example 3

### Bronchiolitis Treatment by Nitric Oxide Inhalation

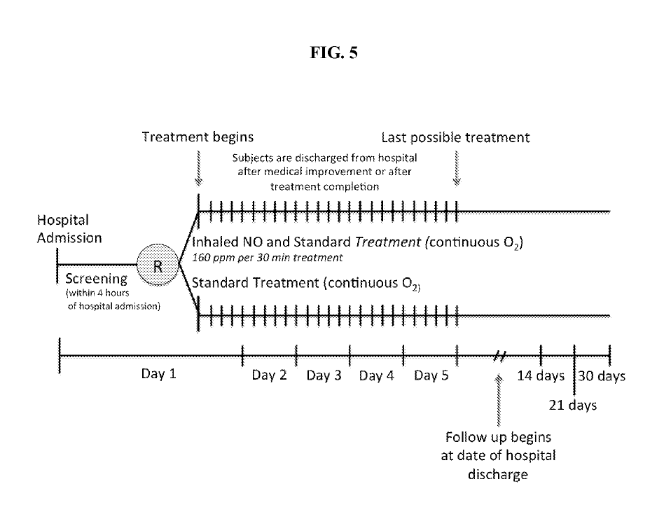

A cohort of human subjects diagnosed with acute bronchiolitis were treated with intermittent inhalation of nitric oxide according to the methods described below and outlined in FIG. 5.

Subjects were screened within 4 hours of admission to the pediatric department. For inclusion in the study, subjects were required to be 2 to 12 months old, diagnosed with acute bronchiolitis, and to have a clinical score of less than nor equal to 10 (see Table 2).

Subjects diagnosed with concomitant diseases such as pneumonia, urinary tract infection or otitis media; methemoglobinemia; chronic lung disease; immune deficiency; or heart disease (including congenital heart disease) were excluded from the study. Subjects with underlying genetic disorders (CF, Down syndrome) or chronic lung diseases (bronchopulmonary dysplasia, primary ciliary dyskinesia, bronchiolitis obliterans), or hypotonia, were also excluded. Other key exclusion criteria were as follows: MetHb greater than 3% at screening, prematurity less than 36 weeks gestational age, received RSV immunoglobulin prophylaxis, history of frequent epistaxis (greater than 1 episode/month), and significant hemoptysis within 30 days (greater than or equal to 5 mL of blood in one coughing episode or greater than 30 mL of blood in a 24 hour period).

Eligible subjects were randomized (1:1) to receive intermittent NO and standard treatment with O₂ (hereafter referred to as NO treatment) or standard treatment with O₂ (hereafter referred to as standard treatment) alone for a treatment period of up to 5 days. During treatment, MetHb and O₂ saturation levels were continuously monitored using a dedicated monitor.

All subjects were required to return to the clinic for assessments (e.g., physical examination and AE/serious adverse event (SAE) questionnaire) on Day 14 (+5 days) and Day 21 (+5 days), from day of admission to the department, with a minimum of 5 days between follow-up visits. In addition, all subjects were contacted by telephone on Day 30 (+5) from day of admission to the department to complete an AE/SAE questionnaire.

TABLE 2
Determination of Clinical Score

| | Respiratory Rate | | | | |
|---|---|---|---|---|---|
| | (Breaths/Minute) | | | SaO₂ | Accessory |
| | Subject < | Subject ≧ | | (Room | Muscle |
| Score | 6 Months | 6 Months | Wheezing | Air) | Use |
| 0 | 40 | 30 | None⁽¹⁾ | ≧95% | None |
| 1 | 41-55 | 31-45 | End expiration | 92-94% | + |
| | | | with | | |
| | | | stethoscope | | |
| 2 | 56-70 | 46-60 | Inspiration and | 90-91% | ++ |
| | | | expiration with | | |
| | | | stethoscope | | |
| 3 | >70 | >60 | Audible | 89% | +++ |
| | | | without | | |
| | | | stethoscope | | |

¹If wheezes not audible due to a minimal air entry, consider score = 3.
Notes:
Clinical score was calculated as the sum of scores given according to each parameter (respiratory rate, wheezing, SaO₂ and accessory muscle use). Mild: ≦5; Moderate: 6-10; Severe: 11-12.
SaO₂ = Oxygen saturation.

Study treatments were as follows:
Investigational treatment: 800 ppm (0.08%) NO with 99.999% nitrogen purity balanced with N₂; delivered by inhalation mask at 160 ppm NO (with a blend of air and O₂ at a minimum concentration of 21% O₂).
Control treatment: O₂ (supplied by the main hospital O₂ system); delivered as 100% O₂ by inhalation mask.

In addition to standard supportive O₂ treatment which was initiated prior to randomization, subjects in the NO group were given five 30 minute inhalations per day of 160 ppm NO, at intervals of 3 to 4 hours, until subject improvement led to a physician decision of fit to discharge, to a maximum of 25 inhalations over 5 consecutive days. Inhalations were administered via hospital face masks (Hospiltak, Unomedical Inc).

All subjects in the control group were managed with standard supportive O₂ treatment in the same way as the active treatment group, but without actual NO administration.

Both treatments, NO and O₂ (NO treatment) as well as O₂ alone (control) were given via the same device, so that the subjects' parents and ward staff would not know what treatment was given. Treatment blindness was maintained by separating between un blinded team members (giving the actual treatment) and blinded team members, and by hiding the NO container and all study-related equipment behind a curtain.

Oxygen for standard care was supplied from the main hospital O₂ system. The O₂ passed through the O₂ blender (BIRD MODEL 03800), followed by an O₂ flow meter. For the control group (O₂ only), the microblender was set do deliver 100% O₂. For the NO treatment group, the O₂ was blended with the air to reach a minimum concentration of 21% in the inhaled 160 ppm NO gas mixture. The blended air/O₂ was supplied to subjects via a Y shape connector attached next to a hospital face mask (Hospiltak, Unomedical Inc).

For subjects in the NO group, the NO concentration (initially 800 ppm) was adjusted by passing through a pressure regulator and indicator (International Biomedical, US, Part No.: 731-9142) and a flow meter (Carefusion US, Model 03800). After the system set up procedure was completed, the NO flow through the flow meter was adjusted before each inhalation to deliver 160 ppm of NO at a total flow of 5 to 15 L/min NO. NO was supplied to the subject via the second arm of the Y shaped connector (specified above) attached next to the face mask. NO, NO₂ and O₂ concentrations delivered to the patient were continuously monitored from a sampling port, using a dedicated monitor (AeroNox International Biomedical, US).

Methemoglobin and O₂ saturation levels were also continuously monitored using a dedicated monitor. In the event of MetHb>5% or O₂ saturation less than 89%, treatment was temporarily discontinued, and measurement repeated 30 minutes later. If value(s) had returned to within the safety threshold, the next inhalation was started according to protocol. In any case of a second episode of MeHb greater than 5%, study treatment was to be permanently discontinued.

Study Endpoints:

The primary study endpoints were safety and tolerability. There were no primary efficacy endpoints.

Primary Endpoints—Safety and Tolerability:
Safety:
   Determine the MetHb percentage associated with inhaled NO
   Determine AEs associated with inhaled NO
Tolerability:
   Proportion of subjects (%) who prematurely discontinued the study or study treatment for any reason
   Proportion of subjects (%) who prematurely discontinued the study treatment due to AEs

Secondary Endpoints—Efficacy:
LOS in hours, measured as time from first inhalation treatment to “fit to discharge”
Time to reach 92% O₂ saturation (in room air) measured as time from first inhalation treatment to first O₂ saturation of 92% sustained to discharge
Time to clinical score of less than or equal to 5 measured as time from first inhalation treatment to clinical score of less than or equal to 5

Analysis Sets:

The following analysis sets were defined for analysis of safety and efficacy:
Intent-to-treat (ITT): Defined as all randomized subjects who received at least one study treatment. This was the main set for analysis of efficacy and safety.
Per-Protocol (PP): Defined as all subjects in the ITT cohort who completed study treatment in compliance with the protocol (i.e., excludes all early termination subjects) and who had no major protocol violations. This was a secondary set for analysis of efficacy.

An additional set for analysis of efficacy was defined post-hoc:
Modified Intent-to-treat (mITT): Defined as all subjects in the ITT, excluding subjects who were prematurely discontinued from treatment and/or or discontinued from the study according to protocol guidelines.

Statistical and Analysis Methods:

Length of hospitalization stay (LOS) was calculated in hours from the first inhalation treatment to “fit to discharge” defined as physician decision to discharge. The fit to discharge time was taken from the last clinical score where applicable, and from a subject's medical chart in special circumstances (i.e., for subjects that did not reach a clinical score of less than or equal to 5 during the study and for subjects that remained in the hospital for suspected bronchiolitis-related incidents).

Time to achieve O₂ saturation of 92% (improvement) leading to discharge was calculated from first treatment to the first time of O₂ saturation of at least 92% before discharge. Time was taken from the clinical score assessments where applicable and from subject daily chart in special circumstances.

Time to clinical score of less than or equal to 5 was calculated from first inhalation to the first time the subject reached a clinical score of less than or equal to 5. For subjects who did not reach a clinical score of less than or equal to 5, the LOS was imputed as time to clinical score less than or equal to 5.

For the analysis of the ITT set, the last observation carried forward (LOCF) approach was used to account for missing data.

All measured variables and derived parameters were tabulated by descriptive statistics. Categorical variables were presented in summary tables including sample size, absolute and relative frequencies, by study group and overall.

Continuous variables were summarized in tables including sample size, arithmetic mean, SD, standard error, median, minimum and maximum by study group.

The following statistical tests were used in the analysis of the data presented in this study:

The Paired T-Test was applied for testing the statistical significance of the changes from baseline for quantitative variables within each study group.

The two-sample T-test or Non-parametric Wilcoxon Rank Sum test or median tests were used as appropriate for analyzing differences between the study groups in quantitative parameters.

The Chi-square test was applied for testing the statistical significance of the differences in frequency of categorical variables between the study groups.

Survival Analysis using a Kaplan-Meier survival function curve was applied for testing the statistical significance of the difference between the study groups in the following endpoints:
LOS, from first inhalation to fit to discharge
Time to achieve 92% Saturation leading to discharge
Time to achieve clinical score less than or equal to 5

The Cox model was applied for comparative analysis of Kaplan-Meier curves. The hazards ratio was estimated via the Cox's regression model.

All tests applied were two-tailed, and a p-value of 5% or less was considered statistically significant. The data was analyzed using the SAS® version 9.1 (SAS Institute, Cary N.C.).

Post-hoc subgroup analyses of subjects with a LOS less than or equal to 24 hours and greater than 24 hours were also conducted for the key post-hoc secondary endpoints. Additional exploratory analyses were also conducted on a subgroups with LOS greater than 36 hours and a subgroup of the 10 most severe subjects (i.e., longest LOS) from each treatment group. These post-hoc analyses were conducted for the following reasons:
Based on preclinical studies, the anti-viral/anti-microbial treatment effect of NO is expected to take at least 24 hours (i.e., 2.5 treatment hours).
Approximately ⅓ of subjects were discharged after less than 24 hours in hospital. Subjects with LOS less than 24 hours were considered as having “very mild disease” and their improvement was likely not related to any treatment.
A longer LOS is expected to correlate with a higher disease severity, and therefore any treatment effect should be more evident in the 3 subgroups LOS greater than 24 hours, LOS greater than 36 hours, and 10 most severe.

The planned sample size was 40 subjects, 20 in each study group. Considering an expected dropout rate of approximately 10%, 44 subjects were planned for recruitment in order to have a sample size of 40 human subjects who completed the study.

Study Subjects:

The study population includes 43 subjects aged 2- to 12-months old with bronchiolitis who required hospitalization at the Soroka University Medical Center in Beer Sheva, Israel. The overall disposition of all subjects screened is summarized in Table 3. A total of 43 subjects were screened and randomized: 21 in the NO group and 22 in the standard care group. Of these, 19/21 (90.5%) subjects in the NO group, and 20/22 (90.9%) in the standard treatment group completed the entire treatment and study duration.

TABLE 3
Subject Disposition (All Subjects Randomized, N = 43)

| | NO and | | | | | |
|---|---|---|---|---|---|---|
| | Standard | | Standard | | | |
| | Treatment (O₂) | | Treatment (O₂) | | All | |
| | (N = 21) | | (N = 22) | | (N = 43) | |
| Parameter | n (%) | | n (%) | | n (%) | |
| Screened | 21 | (100.0%) | 22 | (100.0%) | 43 | (100.0%) |
| Randomized | 21 | (100.0%) | 22 | (100.0%) | 43 | (100.0%) |
| Completed entire | 19 | (90.5%) | 20 | (90.9%) | 39 | (90.7%) |
| treatment and study | | | | | | |
| duration | | | | | | |
| Subjects that discon- | 2 | (9.5%) | 2 | (9.1%) | 4 | (9.3%) |
| tinued study treat- | | | | | | |
| ment or discontinued | | | | | | |
| the study | | | | | | |
| Early discontinua- | 2 | (9.5%) | 1 | (4.5%) | 3 | (7.0%) |
| tion of treatment | | | | | | |
| only | | | | | | |
| AE | 1 | (4.8%)⁽¹⁾ | 0 | (0.0%) | 1 | (2.3%) |
| SAE | 0 | (0.0%) | 1 | (4.5%)⁽²⁾ | 1 | (2.3%) |
| Physician/Investiga- | 1 | (4.8%)⁽³⁾ | 0 | (0.0%) | 1 | (2.3%) |
| tor decision | | | | | | |
| Early discontinua- | 0 | (0.0%) | 1 | (4.5%) | 1 | (2.3%) |
| tion of study | | | | | | |
| Withdrawal of | 0 | (0.0%) | 1 | (4.5%)⁽⁴⁾ | 1 | (2.3%) |
| consent | | | | | | |

¹MetHb > 5% twice in the study (Subject 24).
²This subject experienced an SAE of respiratory failure and was transferred to the pediatric intensive care unit for 2 days before returning back to the pediatric department (Subject 36).
³Request of primary care physician or Investigator (Subject 26).
⁴Subject's parent/legal guardian withdrew consent (Subject 8).
Notes:
AE = Adverse event; SAE =Serious adverse event.

TABLE 4
Summary of Analysis Sets (All Subjects Randomized, N = 43)

| | NO and Standard | | Standard | | | |
|---|---|---|---|---|---|---|
| | Treatment (O₂) | | Treatment (O₂) | | All | |
| | (N = 21) | | (N = 22) | | (N = 43) | |
| Analysis Set | n (%) | | n (%) | | n (%) | |
| ITT | 21 | (100.0%) | 22 | (100.0%) | 43 | (100.0%) |
| mITT⁽¹⁾ | 19 | (90.5%) | 21 | (95.5%) | 40 | (93.0%) |
| pp⁽²⁾ | 19 | (90.5%) | 20 | (90.9%) | 39 | (90.7%) |

¹Excludes Subject 24 (NO group), Subject 26 (NO group) and Subject 8 (Standard Treatment group).
²Excludes Subject 24 (NO group), Subject 26 (NO group), Subject 8 (Standard Treatment group) and Subject 36 (Standard Treatment group).
Notes:
ITT = Intent-to-Treat; mITT = Modified Intent-to-Treat; PP = Per-Protocol.

All randomized subjects (N=43) were included in the ITT which was used for the analysis of safety and was the main analysis set for efficacy. Mean MetHb values were within the normal laboratory reference range for both treatment groups at screening, and ranged from 0.10 to 1.40%; the mean (SD) MetHb level was 0.69 (0.43)% in the NO group and 0.73 (0.30)% in the standard treatment group (see Table 5).

TABLE 5
Summary of Demography and Baseline Characteristics (ITT, N = 43)

| | NO and | | Standard | | p-value | | |
|---|---|---|---|---|---|---|---|
| | Standard | | Treatment | | Chi- | p- | |
| | Treatment | | (O₂) | | square | value | p-value |
| Demographic Variable | (O₂) (N = 21) | | (N = 22) | | Test⁽¹⁾ | T-test⁽²⁾ | Wilcoxin⁽³⁾ |
| Gender (n (%)) | | | | | 0.9065 | — | — |
| Male | 13 | (61.9%) | 14 | (63.6%) | — | — | — |
| Female | 8 | (38.1%) | 8 | (36.4%) | — | — | — |
| Ethnicity (n (%)) | | | | | 0.6502 | — | — |
| Jewish | 5 | (23.8%) | 4 | (18.2%) | — | — | — |
| Bedouin | 16 | (76.2%) | 18 | (81.8%) | — | — | — |
| Age (months) | | | | | | | |
| n | 21 | | 22 | | — | — | |
| Mean (SD) | 4.8 | (2.3) | 5.6 | (2.8) | — | 0.3486 | 0.4627 |
| Median | 4.1 | | 5.5 | | — | — | — |
| Min/max | 2.0/8.7 | | 2.0/11.9 | | — | — | — |
| Weight at screening (g) | | | | | | | |
| n | 21 | | 22 | | — | — | — |
| Mean (SD) | 6.6 | (1.6) | 6.8 | (1.8) | — | 0.8114 | 0.9807 |
| Median | 6.5 | | 6.5 | | — | — | — |
| Min/max | 3.6/10.0 | | 4.4/11.0 | | — | — | — |
| Gestational age at birth | | | | | | | |
| (weeks) | | | | | | | |
| n | 21 | | 22 | | — | — | — |
| Mean (SD) | 38.9 | (1.6) | 39.3 | (1.1) | — | 0.2776 | 0.2363 |
| Median | 39.0 | | 40.0 | | — | — | — |
| Min/max | 36.0/42.0 | | 36.0/40.0 | | — | — | — |
| MetHb at screening (%) | | | | | | | |
| n | 21 | | 21 | | — | — | — |
| Mean (SD) | 0.69 | (0.43) | 0.73 | (0.30) | — | 0.7106 | — |
| Median | 0.80 | | 0.70 | | — | — | — |
| Min/max | 0.10/1.40 | | 0.20/1.20 | | — | — | — |
| Clinical score at | | | | | | | |
| screening | | | | | | | |
| n | 21 | | 22 | | — | — | — |
| Mean (SD) | 7.86 | (1.11) | 8.09 | (1.27) | — | 0.5244 | 0.4600 |

| | | | | | |
|---|---|---|---|---|---|
| Median | 7.00 | 8.00 | — | — | — |
| Min/Max | 7.00/10.00 | 6.00/8.00 | — | — | — |

¹Chi-square test for testing significance of difference in proportions between the study groups.
²T-test (unpaired) for difference in means between the study groups.
³Non-parametric Wilcoxon-Mann-Whitney Rank sum test for difference in means between the study groups.
Notes:
ITT = Intent-to-Treat; Max = Maximum; Min = Minimum; MetHb = Methemoglobin; ND = Not determined; SD = Standard deviation.

The mean/median clinical score was comparable between groups (see Table 5) and all subjects had a moderate severity of bronchiolitis. The majority of subjects in both treatment groups had normal physical exam results at screening/baseline (i.e., 76.2% in the NO group and 81.8% in the standard treatment group) except for pyrexia for some subjects (maximum temp 39.5° C.).

A summary of viruses detected in nasal washes prior to first treatment is shown in Table 6. In both treatment groups, the majority of subjects were positive for RSV (71.4% in the NO group and 63.6% in the standard treatment group).

TABLE 6
Summary of Viruses Detected in Nasal Washes Prior to First
Treatment, Number and Percentage of Subjects (ITT, N = 43)

| | NO and Standard | | Standard | |
|---|---|---|---|---|
| | Treatment (O₂) | | Treatment (O₂) | |
| | (N = 21) | | (N = 22) | |
| Viruses Detected⁽¹⁾ | n (%) | | n (%) | |
| Adenovirus | 0 | (0.0%) | 2 | (9.1%) |
| Corona virus | 2 | (9.5%) | 2 | (9.1%) |
| Coronoa-NL63 | 0 | (0.0%) | 1 | (4.5%) |
| Coronoa-OC43 | 2 | (9.5%) | 1 | (4.5%) |
| Metapneumovirus | 2 | (9.5%) | 1 | (4.5%) |
| Influenza A | 0 | (0.0%) | 4 | (18.2%) |
| Influenza A H1N1 | 0 | (0.0%) | 2 | (9.1%) |
| RSV | 15 | (71.4%) | 14 | (63.6%) |

¹Virology results reported in this table are based on results from RT-PCR, with the exception of 1 RSV sample for which serology was used. Individual subjects may have had more than virus type.
Notes:
ITT = Intent-to-Treat; RSV = Respiratory Syncytial Virus; RT-PCR = Reverse transcriptase polymerase chain reaction.

Demographics and baseline characteristics were also compared for subgroups with a LOS greater than 24 hours and less than or equal to 24 hours (see Table 7). No statistically significant differences were observed for any of the parameters, either between treatment groups within a given sub population or between sub-populations for treatment groups combined.

TABLE 7
Demographic and Baseline Characteristics of Subjects with a LOS ≦
24 Hours and > 24 Hours (ITT, N = 43)

| | LOS ≦ 24 Hours (N = 16) | | | | LOS > 24 Hours (N = 27) | | | |
|---|---|---|---|---|---|---|---|---|
| | NO and | | | | NO and | | | |
| | Standard | | Standard | | Standard | | Standard | |
| | Treatment | | Treatment | | Treatment | | Treatment | |
| Demographic | (O₂) | | (O₂) | | (O₂) | | (O₂) | |
| Variable | (N = 6) | | (N = 10) | | (N = 15) | | (N = 12) | |
| Gender (n (%)) | | | | | | | | |
| Male | 3 | (50.0%) | 8 | (80.0%) | 10 | (66.7%) | 6 | (50.0%) |
| Female | 3 | (50.0%) | 2 | (20.0%) | 5 | (33.3%) | 6 | (50.0%) |
| Age (months) | | | | | | | | |
| n | 6 | | 10 | | 15 | | 12 | |
| Mean (SD) | 5.49 | (2.31) | 5.42 | (2.66) | 4.57 | (2.27) | 5.70 | (3.06) |
| Median | 5.45 | | 5.14 | | 4.11 | | 5.86 | |
| Min/max | 2.86/8.05 | | 1.97/9.95 | | 2.04/8.67 | | 2.07/11.93 | |
| Body temperature | | | | | | | | |
| at screening (° C.) | | | | | | | | |
| n | 6 | | 10 | | 15 | | 12 | |
| Mean (SD) | 37.20 | (0.52) | 37.64 | (0.88) | 37.57 | (0.84) | 37.36 | (0.91) |
| Median | 37.20 | | 37.50 | | 37.50 | | 37.20 | |
| Min/max | 36.30/37.80 | | 36.50/39.50 | | 36.40/38.80 | | 36.40/39.40 | |
| Clinical score at | | | | | | | | |
| screening | | | | | | | | |
| n | 6 | | 10 | | 15 | | 12 | |
| Mean (SD) | 7.67 | (1.21) | 8.30 | (1.25) | 7.93 | (1.10) | 7.92 | (1.31) |

| | | | | |
|---|---|---|---|---|
| Median | 7.00 | 8.00 | 7.00 | 8.00 |
| Min/max | 7.00/10.0 | 6.00/10.0 | 7.00/10.0 | 6.00/10.0 |

Notes:
ITT = Intent-to-Treat; Max = Maximum; Min = Minimum; MetHb = Methemoglobin; ND = Not determined; SD = Standard deviation.

Exposure and Treatment Compliance:

Subjects were given five 30-minute inhalations per day of NO (NO group) or O₂ alone (standard treatment, control group) at intervals of 3 to 4 hours, until subject improvement led to a decision of fit to discharge, to a maximum of 25 inhalations per subject over 5 consecutive days.

Given the study design (inhalations administered in hospital), compliance was good in both treatment groups. Three subjects in each treatment group missed 1 or more planned doses, and of the doses administered to subjects, the majority of inhalations (>96% in each treatment group) were complete 30 minute inhalations (see Table 8).

The mean number of inhalations was lower in the NO group (7.4) as compared to the control group (9.0) although the difference between groups was not statistically significant. In the NO group, the maximum number of treatments was 16, compared to 25 in the standard treatment group, and as shown in Table 7, the number of subjects at each treatment number was greater in the standard treatment group from Treatment Number 11 onwards.

TABLE 8
Summary of Exposure and Treatment Compliance (ITT, N = 43)

| | Number of Inhalations | | | |
|---|---|---|---|---|
| | | | Standard | |
| | NO and Standard | | Treatment | |
| | Treatment (O₂) | | (O₂) | |
| | (N = 21) | | (N = 22) | |
| Total number of | 156 | | 198 | |
| inhalations received⁽¹⁾ | | | | |
| Mean (SD) | 7.43 | (3.19) | 9.00 | (6.53) |
| Median | 7.00 | | 6.50 | |
| Min/Max | 2/16 | | 3/25 | |
| Total number of complete | 150 | | 195 | |
| inhalations received⁽²⁾ | | | | |
| Subjects per treatment | n | (%) | n | (%) |
| number | | | | |
| 1 | 21 | (100.0%) | 22 | (100.0%) |
| 2 | 20 | (95.2%) | 21 | (95.5%) |
| 3 | 19 | (90.5%) | 20 | (90.9%) |
| 4 | 19 | (90.5%) | 19 | (86.4%) |
| 5 | 16 | (76.2%) | 18 | (81.8%) |
| 6 | 14 | (66.7%) | 13 | (59.1%) |
| 7 | 13 | (61.9%) | 11 | (50.0%) |
| 8 | 11 | (52.4%) | 9 | (40.9%) |
| 9 | 8 | (38.1%) | 8 | (36.4%) |
| 10 | 7 | (33.3%) | 8 | (36.4%) |
| 11 | 3 | (14.3%) | 6 | (27.3%) |
| 12 | 1 | (4.8%) | 6 | (27.3%) |
| 13 | 1 | (4.8%) | 5 | (22.7%) |
| 14 | 1 | (4.8%) | 5 | (22.7%) |
| 15 | 1 | (4.8%) | 4 | (18.2%) |
| 16 | 1 | (4.8%) | 4 | (18.2%) |
| 17 | 0 | (0.0%) | 3 | (13.6%) |
| 18 | 0 | (0.0%) | 2 | (9.1%) |
| 19 | 0 | (0.0%) | 2 | (9.1%) |
| 20 | 0 | (0.0%) | 2 | (9.1%) |
| 21 | 0 | (0.0%) | 2 | (9.1%) |
| 22 | 0 | (0.0%) | 2 | (9.1%) |
| 23 | 0 | (0.0%) | 2 | (9.1%) |
| 24 | 0 | (0.0%) | 2 | (9.1%) |
| 25 | 0 | (0.0%) | 2 | (9.1%) |

¹Includes all treatments (30-minute inhalations) for which a start and stop time were recorded, including treatments discontinued prior to the full 30 minutes.
²Excludes any inhalations which were discontinued prior to the full 30 minute specified per protocol.
Notes:
The ITT includes 2 subjects who were prematurely withdrawn from treatment due to AEs and 2 subjects who were prematurely withdrawn both from treatment and the study due to withdrawal of consent/physician decision.
Max = Maximum; Min = Minimum; SD = Standard deviation.

Primary Safety Endpoints—MetHb Percentage Associated with Inhaled NO:

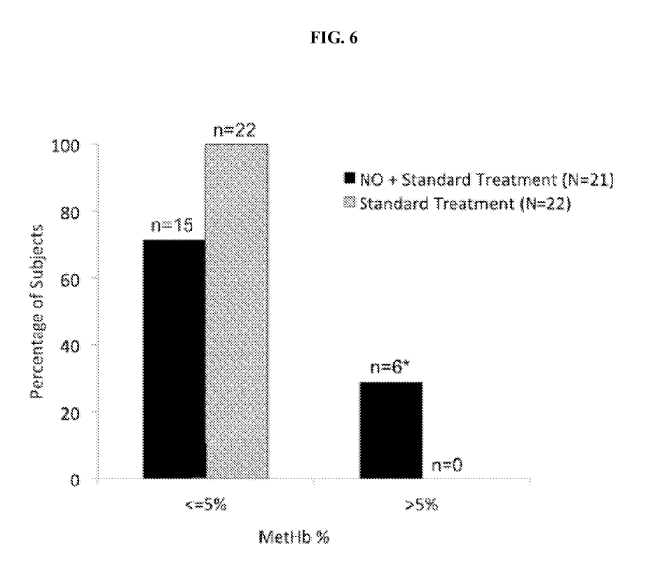

The percentage of subjects with MetHb>5% or <5% during the study treatment period is shown in FIG. 6. There were no subjects in the standard treatment with MetHb greater than 5%. In the NO group, 6 (28.6%) subjects had any MetHb measurement greater than 5% during the study treatment period, and 3 of these subjects had more than one MetHb greater than 5%. The maximum MetHb was 5.6 in one subject in the NO group (Subject 44); this subject was not discontinued from study treatment.

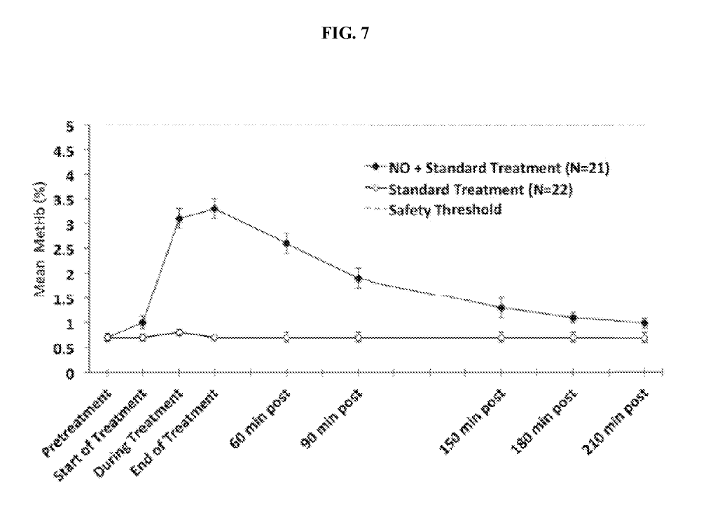

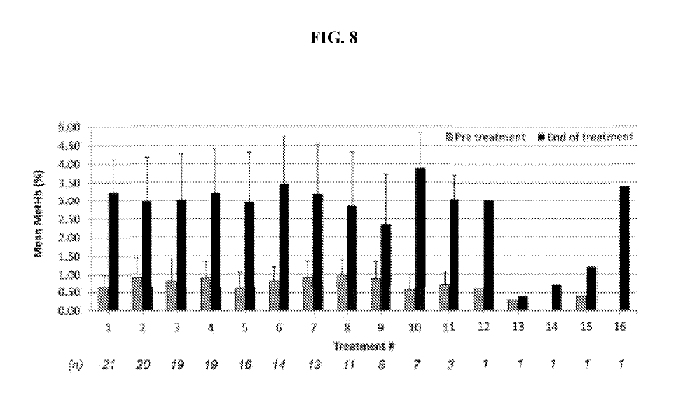

A plot of MetHb levels monitored before, during, and after treatment is shown in FIG. 7. In the NO group, MetHb increased during treatment, with peak values at end of treatment, and then gradually declined, approaching pre-treatment levels within approximately 3 hours. As shown in FIG. 8, when comparing pre-treatment and end of treatment MetHb levels for each treatment number in the study, no “cumulative” effect on MetHb levels was observed over the study treatment period.

Efficacy Evaluation—Length of Stay:

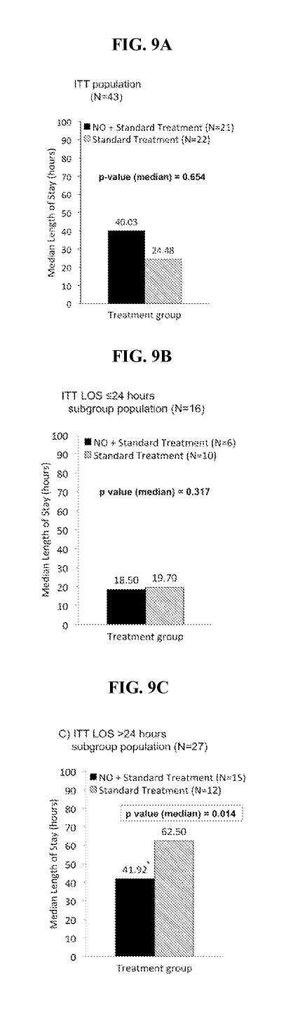

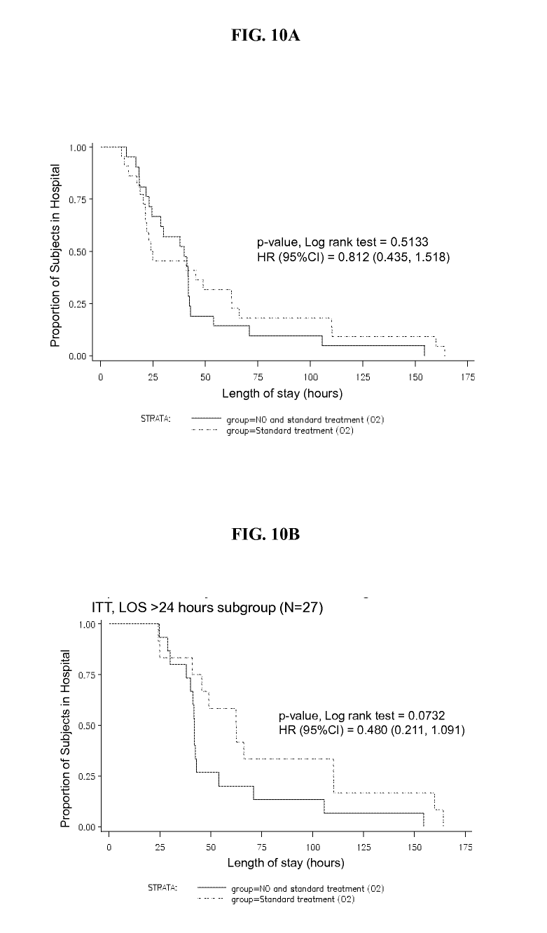

A summary of the analyses of mean and median LOS for all subjects in the ITT, and according to subgroups based on LOS greater than 24 hours and LOS less than or equal to 24 hours, is shown in Table 9 and FIG. 9, and a summary of the corresponding Kaplan-Meier analyses is shown in FIG. 10.

TABLE 9
Mean and Median LOS According to Treatment and Subgroup (ITT)

| | | | p-value for | | | p-value for |
|---|---|---|---|---|---|---|
| | LOS, hours | | difference | LOS, hours | | difference |
| | Mean (SD) | | between | Median | | between |
| | | Standard | means | | Standard | median |
| Subgroup | NO | Treatment | (Wilcoxon) | NO | Treatment | values |
| All (N = 43) | 43.34 | 49.98 | 0.8563 | 40.03 | 24.48 | 0.6535 |
| | (32.95) | (46.23) | | | | |
| ≦24 hours | 18.52 | 17.92 | 0.9575 | 18.50 | 19.70 | 0.3173 |
| (N = 16) | (3.84) | (4.72) | | | | |
| >24 hours | 53.27 | 76.69 | 0.1363 | 41.92 | 62.50 | 0.0142* |
| (N = 27) | (34.29) | (48.46) | | | | |

*Indicates statistical significance at p < 0.05.
Notes:
ITT = Intent-to-Treat;
LOS = Length of stay;
SD = Standard deviation.

In the ITT (N=43), there was a shorter mean LOS in the NO group compared to the standard treatment group; however, the differences between treatment groups were not statistically significant. Variability between subjects was high as evidenced in the SD values. In the NO group, the mean (SD) LOS was 43.34 (32.95) hours compared to 49.98 (46.23) hours in the standard treatment group (p=0.856), and the median LOS was 40.03 hours compared to 24.48 hours, respectively (p=0.654). Results of the Kaplan Meier analysis (see FIG. 10A) were also not statistically significant (hazard ratio (HR)=0.812, 95% CI: 0.435, 1.518; log rank p value=0.513).

When the ITT was examined according to subgroups based on LOS greater than 24 hours and LOS less than or equal to 24 hours, the median LOS was shown to be statistically significantly shorter in the NO group (41.92 hours) compared to the standard treatment group (62.50 hours) (p=0.014) for subjects with LOS greater than 24 hours (see Table 9 and FIG. 9C). Kaplan Meier analysis also showed a trend in favor of the NO group for subjects with LOS greater than 24 hours, although the results were not statistically significant (HR=0.480, 95% CI: 0.211, 1.091; log rank p value=0.073) (see FIG. 10B). In the subgroup with LOS less than or equal to 24 hours, mean and median LOS were similar between treatment groups, with no statistically significant differences observed.

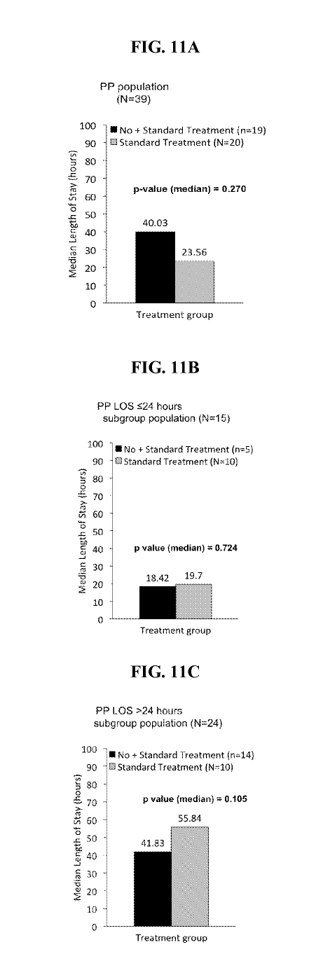

As shown in FIG. 11 and Table 10, results for analysis of the PP set (N=39) were similar to those observed for the ITT (N=43). Although there was a shorter mean LOS in the NO group compared to the standard treatment group, the differences between treatment groups were not statistically significant. In the NO group, the mean (SD) LOS was 38.79 (21.52) hours compared to 43.86 (40.97) hours in the standard treatment group (p=0.531), and the median LOS was 40.03 hours compared to 23.56 hours, respectively (p=0.270). Results of the Kaplan-Meier analysis were also not statistically significant for the PP (HR=0.866, 95% CI: 0.444, 1.688; log rank p value=0.671).

When the PP was examined according to subgroups based on LOS greater than 24 hours and LOS less than or equal to 24 hours, both the mean and the median LOS were shorter in the NO group compared to the standard treatment group for subjects with LOS greater than 24 hours, although the differences between groups were not statistically significant (see Table 10 and FIG. 11C). Kaplan Meier analysis also showed a trend in favor of the NO group for subjects with LOS greater than 24 hours, although the results were not statistically significant (HR=0.418, 95% CI: 0.164, 1.062; log rank p value=0.059). In the subgroup with LOS less than or equal to 24 hours, mean and median LOS were similar between treatment groups, with no statistically significant differences observed (see Table 10 and FIG. 11B).

TABLE 10
Mean and Median LOS According to Treatment and Subgroup (PP)

| | | | | | | p-value |
|---|---|---|---|---|---|---|
| | | | p-value for | | | for |
| | LOS, hours | | difference | LOS, hours | | difference |
| | Mean (SD) | | between | Median | | between |
| | | Standard | means | | Standard | median |
| Subgroup | NO | Treatment | (Wilcoxon) | NO | Treatment | values |
| All (N = 39) | 38.79 | 43.86 | 0.5310 | 40.03 | 23.56 | 0.2700 |
| | (21.52) | (40.97) | | | | |
| ≦24 hours | 18.50 | 17.92 | 0.8569 | 18.42 | 19.70 | 0.7237 |
| (N = 15)⁽¹⁾ | (4.29) | (4.72) | | | | |
| >24 hours | 46.03 | 69.81 | 0.1820 | 41.83 | 55.84 | 0.1050 |
| (N = 24) | (20.52) | (45.01) | | | | |

*Indicates statistical significance at p < 0.05.
⁽¹⁾For the subgroup of subjects with LOS ≦24 hours, the populations for the PP and mITT are the same in this study.
Notes:
LOS = Length of stay;
mITT = Modified Intent-to-Treat;
PP = Per-Protocol;
SD = Standard deviation.

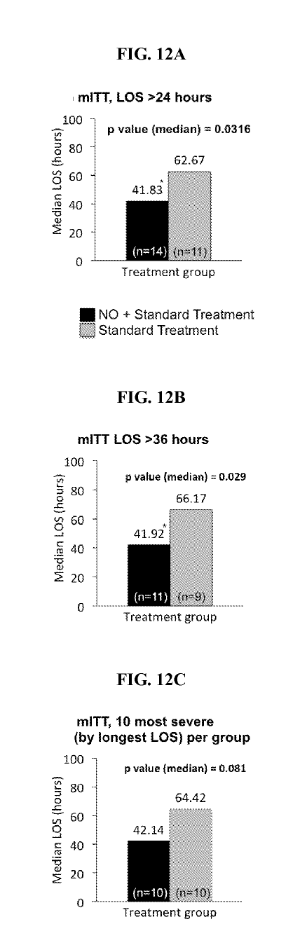

For the subgroup of subjects with LOS greater than 24 hours, results observed for analysis of the mITT (N=25) were similar to those observed for the ITT (N=27) and PP (N=24). For subjects with LOS greater than 24 hours (mITT), a statistically significant difference was demonstrated between the median LOS values (i.e., 41.83 hours compared to 62.67 hours for the NO and standard treatment groups, respectively (p=0.032)) (see FIG. 12A). Additionally, a statistically significant difference in favor of the NO group was seen by Kaplan-Meier analysis (HR=0.357, 95% CI: 0.140, 0.913; log rank p value=0.025).

Additional exploratory analyses were conducted on the mITT to examine the differences between subgroups with LOS greater than 36 hours and for the 10 most severe subjects (i.e., longest LOS) from each treatment group (see FIG. 12B and FIG. 12C). For subjects with LOS greater than 36 hours, the median LOS was 41.92 versus 66.17 hours (p=0.029) for the NO and standard treatment groups, respectively, and for the 10 most severe subjects from each group, the median LOS was 42.14 versus 64.42 hours (p=0.081) for the NO and standard treatment groups, respectively. It should be noted, however, that interpretation of these results is limited by the low number of subjects per subgroup.

Time to First 92% 02 Saturation Sustained to Discharge:

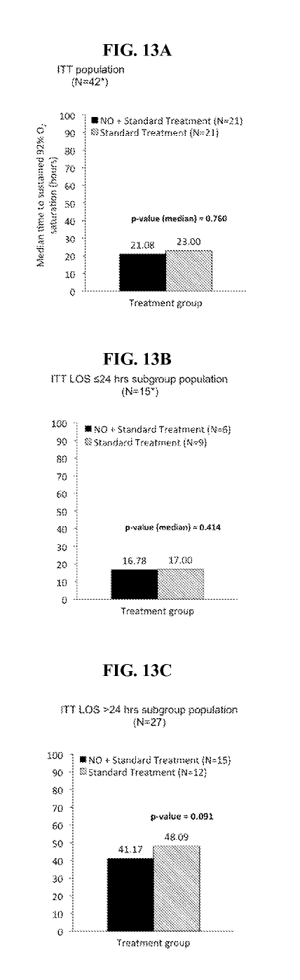

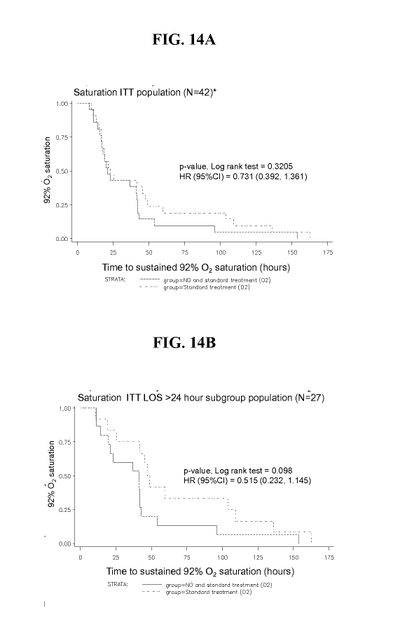

A summary of the analyses of mean and median time to first 92% 02 saturation (improvement) sustained to discharge is shown for all subjects in the ITT, and according to subgroups based on LOS greater than 24 hours and LOS less than or equal to 24 hours, in Table 11 and FIG. 13, and a summary of the corresponding Kaplan-Meier analyses is shown in FIG. 14.

In the ITT (N=42), there was a shorter mean time to sustained 92% O₂ saturation in the NO group compared to the standard treatment group; however, the differences between groups were not statistically significant. In the NO group, the mean (SD) time was 35.50 (33.73) hours compared to 45.75 (44.43) hours in the standard treatment group (p=0.517), and the median time was 21.08 hours compared to 23.00 hours, respectively (p=0.760). The trend towards a lower time to sustained 92% O₂ saturation in the NO group was also demonstrated by Kaplan-Meier analysis (see FIG. 14A), although statistical significance was not reached (HR=0.731, 95% CI: 0.392, 1.361; log rank p value=0.321).

A larger difference between treatment groups (in favor of NO) was seen for the subset of subjects with LOS greater than 24 hours, but differences between treatment groups were not statistically significant (see Table 11 and FIG. 13B). Kaplan Meier analysis also showed a trend in favor of the NO group for subjects with LOS greater than 24 hours, although the results were not statistically significant (HR=0.515, 95% CI: 0.232, 1.145; log rank p value=0.098) (see FIG. 14B). In the subgroup with LOS less than or equal to 24 hours, mean and median LOS were similar between treatment groups, with no statistically significant differences observed.

TABLE 11
Mean and Median Time to First 92% O₂ Saturation Sustained to
Discharge, According to Treatment and Subgroup (ITT, N = 43)

| | | | p-value for | | | p-value for |
|---|---|---|---|---|---|---|
| | Time in hours | | difference | Time in hours | | difference |
| | Mean (SD) | | between | Median | | between |
| | | Standard | means | | Standard | median |
| Subgroup | NO | Treatment | (Wilcoxon) | NO | Treatment | values |
| All (N = 42)⁽¹⁾ | 35.50 | 45.75 | 0.5167 | 21.08 | 23.00 | 0.7604 |
| | (33.73) | (44.43) | | | | |
| ≦24 hours | 15.86 | 16.77 | 0.8621 | 16.78 | 17.00 | 0.4142 |
| (N = 15)⁽¹⁾ | (3.90) | (3.89) | | | | |
| >24 hours | 43.35 | 67.49 | 0.1142 | 41.17 | 48.09 | 0.0910 |
| (N = 27) | (37.27) | (48.74) | | | | |

⁽¹⁾Excludes Subject 29 (standard treatment group) as he was admitted with 92% saturation.
Notes:
ITT = Intent-to-Treat;
SD = Standard deviation.

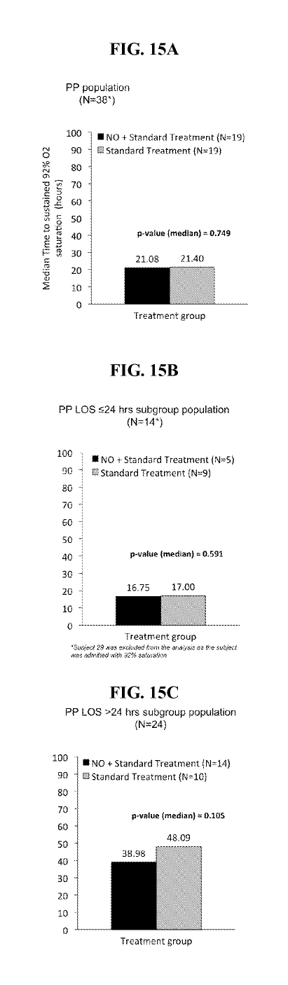

As shown in FIG. 15 and Table 12, results for analysis of the PP set (N=38) were similar to those observed for the ITT (N=42). Although there was a shorter mean time to sustained 92% O₂ saturation in the NO group compared to the standard treatment group, the differences between treatment groups were not statistically significant. In the NO group, the mean (SD) time was 30.17 (21.06) hours compared to 41.22 (41.46) hours in the standard treatment group (p=0.582), and the median time was 21.08 hours compared to 21.40 hours, respectively (p=0.749). Results of the Kaplan-Meier analysis were also not statistically significant for the PP (HR=0.660, 95% CI: 0.334, 1.302; log rank p value=0.227).

When the PP was examined according to subgroups based on LOS greater than 24 hours and LOS less than or equal to 24 hours, both the mean and the median time to sustained 92% O₂ saturation were shorter in the NO group compared to the standard treatment group for subjects with LOS greater than 24 hours, although the differences between groups were not statistically significant (see Table 12 and FIG. 15C). Kaplan Meier analysis showed a statistically significant difference in favor of the NO group for subjects with LOS greater than 24 hours, (HR=0.358, 95% CI: 0.139, 0.921; log rank p value=0.028). In the subgroup with LOS less than or equal to 24 hours, mean and median LOS were similar between treatment groups, with no statistically significant differences observed (see Table 12 and FIG. 15B).

TABLE 12
Mean and Median Time to First 92% O₂ Saturation Sustained to
Discharge, According to Treatment and Subgroup (PP)

| | | | | | | p-value |
|---|---|---|---|---|---|---|
| | | | p-value for | | | for |
| | Time in hours | | difference | Time in hours | | difference |
| | Mean (SD) | | between | Median | | between |
| | | Standard | means | | Standard | median |
| Subgroup | NO | Treatment | (Wilcoxon) | NO | Treatment | values |
| All (N = 38)⁽¹⁾ | 30.17 | 41.22 | 0.5824 | 21.08 | 21.40 | 0.7489 |
| | (21.06) | (41.46) | | | | |
| ≦24 hours | 15.32 | 16.77 | 0.6024 | 16.75 | 17.00 | 0.5909 |
| (N = 14)^{(1,2)} | (4.09) | (3.89) | | | | |
| >24 hours | 35.47 | 63.22 | 0.0975 | 38.98 | 48.09 | 0.1050 |
| (N = 24) | (22.22) | (47.85) | | | | |

⁽¹⁾Excludes Subject 29 (standard treatment group) as he was admitted with 92% saturation.
⁽²⁾For the subgroup of subjects with LOS ≦24 hours, the populations for the PP and mITT are the same in this study.
Notes:
mITT = Modified Intent-to-Treat;
PP = Per-Protocol;
SD = Standard deviation.

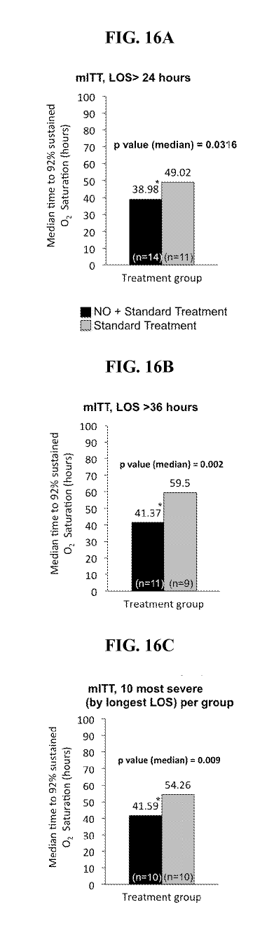

In the mITT, the median time to first 92% O₂ saturation sustained to discharge was shown to be statistically significantly shorter in the NO group (38.98 hours) compared to the standard treatment group (49.02 hours) (p=0.032) for subjects with LOS greater than 24 hours (see FIG. 16A). Additionally, a statistically significant difference in favor of the NO group was seen by Kaplan-Meier analysis (HR=0.308, 95% CI: 0.119, 0.797; log rank p value=0.011).

Additional exploratory analyses were conducted on the mITT to examine the differences between subgroups with LOS greater than 36 hours and for the 10 most severe subjects (i.e., longest LOS) from each treatment group (see FIG. 16). For subjects with LOS greater than 36 hours, the median time to 92% O₂ saturation sustained to discharge was 41.37 versus 59.50 hours (p=0.002) for the NO and standard treatment groups, respectively, and for the 10 most severe subjects from each group, the median time was 41.59 versus 54.26 hours (p=0.009) for the NO and standard treatment groups, respectively. It should be noted, however, that interpretation of these results is limited by the low number of subjects per subgroup.

Time to Clinical Score of Less than or Equal to 5:

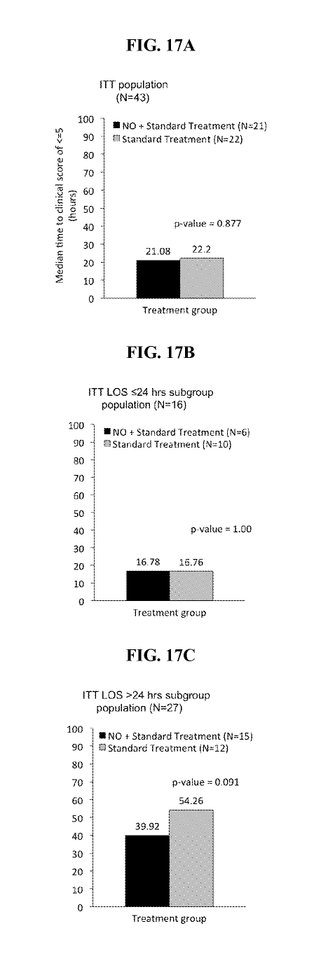

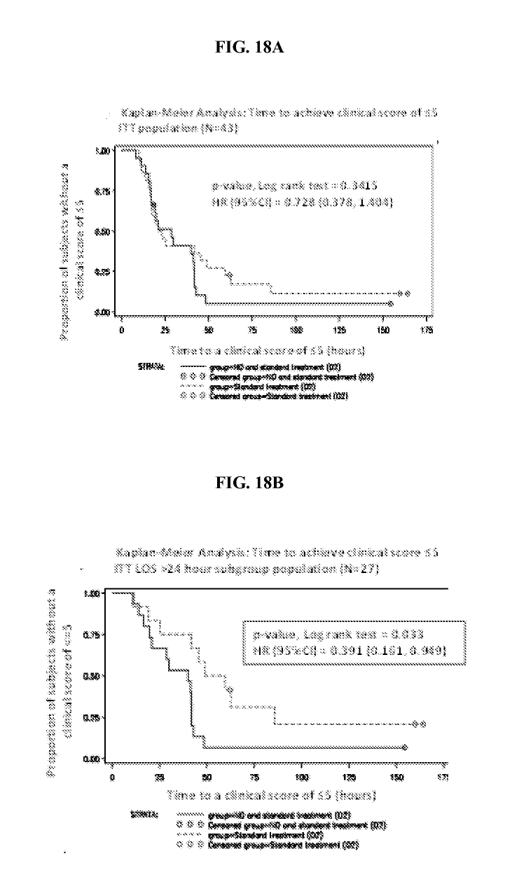

A summary of the analyses of mean and median time to clinical score less than or equal to 5 (improvement) is shown for all subjects in the ITT, and according to subgroups based on LOS greater than 24 hours and LOS less than or equal to 24 hours, in Table 13 and FIG. 17, and a summary of the corresponding Kaplan-Meier analyses is shown in FIG. 18.

In the ITT (N=43), there was a shorter mean time in the NO group compared to the standard treatment group; however, the differences between groups were not statistically significant. In the NO group, the mean (SD) time to clinical score of less than or equal to 5 was 32.83 (30.61) hours compared to 43.10 (43.91) hours in the standard treatment group (p=0.621), and the median time was 21.08 hours compared to 22.20 hours, respectively (p=0.877). The trend towards a lower time to clinical score of less than or equal to 5 in the NO group was also demonstrated by Kaplan-Meier analysis (see FIG. 18A), although statistical significance was not reached (HR=0.728, 95% CI: 0.378, 1.404; log rank p value=0.342).

TABLE 13
Mean and Median Time to Clinical Score ≦5, According to Treatment and
Subgroup (ITT, N = 43)

| | | | | | | p-value |
|---|---|---|---|---|---|---|
| | | | p-value for | | | for |
| | Time in hours | | difference | Time in hours | | difference |
| | Mean (SD) | | between | Median | | between |
| | | Standard | means | | Standard | median |
| Subgroup | NO | Treatment | (Wilcoxon) | NO | Treatment | values |
| All (N = 43) | 32.83 | 43.10 | 0.6210 | 21.08 | 22.20 | 0.8774 |
| | (30.61) | (43.91) | | | | |
| ≦24 hours | 15.86 | 16.24 | 1.0000 | 16.78 | 16.76 | 1.0000 |
| (N = 16) | (3.90) | (4.04) | | | | |
| >24 hours | 39.62 | 65.48 | 0.0649 | 39.92 | 54.26 | 0.0910 |
| (N = 27) | (34.07) | (49.66) | | | | |

Notes:
ITT = Intent-to-Treat;
SD = Standard deviation.

The same trend was seen in the mean and median time to clinical score less than or equal to 5 for the subgroup of ITT subjects with LOS greater than 24 hours, with a larger apparent difference between treatment groups, although statistical significance was not reached (see Table 13). Based on Kaplan-Meier analysis of subjects with LOS greater than 24 hours, a statistically significant difference was seen in favour of the NO group (HR=0.391, 95% CI: 0.161, 0.949; log rank p value=0.033) (see FIG. 18B). In the subgroup with LOS less than or equal to 24 hours, mean and median LOS were similar between treatment groups, with no statistically significant differences observed.

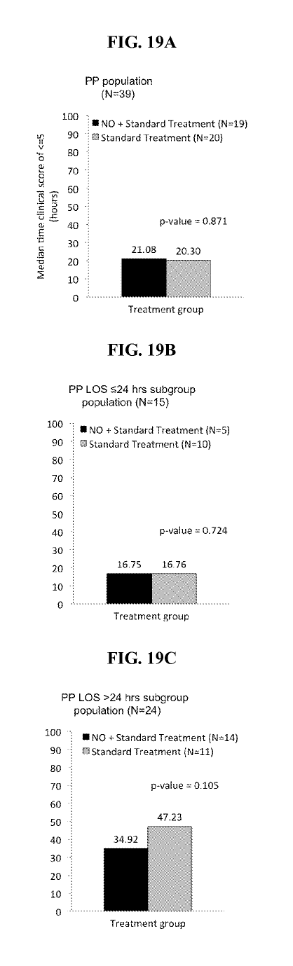

As shown in FIG. 19 and Table 14, results for analysis of the PP set (N=39) were similar to those observed for the ITT (N=43). Although there was a shorter mean time to clinical score less than or equal to 5 in the NO group compared to the standard treatment group, the differences between treatment groups were not statistically significant. In the NO group, the mean (SD) time was 27.18 (13.17) hours compared to 36.30 (36.68) hours in the standard treatment group (p=0.813), and the median time was 21.08 hours compared to 20.30 hours, respectively (p=0.871). Results of the Kaplan-Meier analysis were also not statistically significant for the PP (HR=0.663, 95% CI: 0.332, 1.324; log rank p value=0.241).

When the PP was examined according to subgroups based on LOS greater than 24 hours and LOS less than or equal to 24 hours, both the mean and the median time to clinical score less than or equal to 5 were shorter in the NO group compared to the standard treatment group for subjects with LOS greater than 24 hours, although the differences between groups were not statistically significant (see Table 14 and FIG. 19C). Kaplan Meier analysis showed a statistically significant difference in favor of the NO group for subjects with LOS greater than 24 hours, (HR=0.273, 95% CI: 0.093, 0.799; log rank p value=0.013). In the subgroup with LOS less than or equal to 24 hours, mean and median LOS were similar between treatment groups, with no statistically significant differences observed (see Table 14 and FIG. 19B).

TABLE 14
Mean and Median Time to Clinical Score ≦5, According
to Treatment and Subgroup (PP)

| | | | | | | p-value |
|---|---|---|---|---|---|---|
| | | | p-value for | | | for |
| | Time in hours | | difference | Time in hours | | difference |
| | Mean (SD) | | between | Median | | between |
| | | Standard | means | | Standard | median |
| Subgroup | NO | Treatment | (Wilcoxon) | NO | Treatment | values |
| All (N = 39) | 27.18 | 36.30 | 0.8125 | 21.08 | 20.30 | 0.8711 |
| | (13.17) | (36.68) | | | | |
| ≦24 hours | 15.32 | 16.24 | 0.7638 | 16.75 | 16.76 | 0.7237 |
| (N = 15)⁽¹⁾ | (4.09) | (4.04) | | | | |
| >24 hours | 31.41 | 56.36 | 0.0975 | 34.92 | 47.23 | 0.1050 |
| (N = 24) | (12.72) | (43.94) | | | | |

⁽¹⁾For the subgroup of subjects with LOS ≦24 hours, the populations for the PP and mITT are the same in this study.
Notes:
mITT = Modified Intent-to-Treat;
PP = Per-Protocol;
SD = Standard deviation.

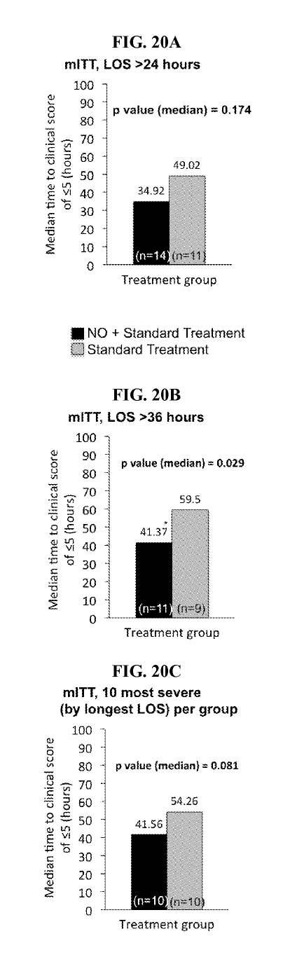

Similar results were observed in the analysis of mITT subjects with LOS greater than 24 hours. The median time to clinical score less than or equal to 5 was 34.92 hours in the NO group and 49.02 hours in the standard treatment group (p=0.174) (see FIG. 20A). Based on Kaplan Meier analysis, a statistically significant difference was demonstrated in favor of the NO group (HR=0.238, 95% CI: 0.081, 0.699; log rank p value=0.005).

Additional exploratory analyses were conducted on the mITT to examine the differences between subgroups with LOS greater than 36 hours and for the 10 most severe subjects (i.e., longest LOS) from each treatment group (see FIG. 20B and FIG. 20C). For subjects with LOS greater than 36 hours, the median time to clinical score less than or equal to 5 was 41.37 versus 59.50 hours (p=0.029) for the NO and standard treatment groups, respectively, and for 10 most severe subjects (based on LOS) from each group, the median time was 41.56 versus 54.26 hours (p=0.081) for the NO and standard treatment groups, respectively. It should be noted, however, that interpretation of these results is limited by the low number of subjects per subgroup.

Conclusion:

In this study of forty-three 2- to 12-month old bronchiolitis subjects, the safety and tolerability of treatment with inhalations of 160 ppm NO for 30 minutes, 5 times daily, for up to 5 consecutive days (in addition to standard 02 treatment) was comparable to standard treatment only.

The overall incidence of AEs was similar between treatment groups, with 10 (47.6%) subjects in the NO group and 13 (59.1%) subjects in the standard treatment group reporting at least 1 AE. Serious adverse events were reported by 4 subjects in each group and there were no deaths during the study.

The percentage of subjects with any MetHb greater than 5% during the study (primary safety endpoint) was 28.6% (6 subjects) in the NO group and 0.0% in the standard treatment group. Three subjects in the NO group had more than one MetHb measurement greater than 5%. Mean MetHb levels were significantly higher in the NO group during treatment but quickly returned to baseline values after treatment stopped. There was no cumulative effect of MetHb exposure during the study, and the maximum MetHb level reported was 5.6% in one subject in the NO group.

There were no primary efficacy analyses conducted in this study and the study was not powered for efficacy. Interpretation of results is limited by the low number of subjects per group. Based on secondary and exploratory analyses, no statistically significant differences were seen between treatment groups for the main efficacy set (ITT, all subjects). However, in a subgroup of subjects with LOS greater than 24 hours, a statistically significant treatment benefit of NO versus standard treatment was demonstrated with respect to a shorter LOS, shorter time to sustained O₂ saturation, and shorter time to clinical score less than or equal to 5.

### Example 4

### Cystic Fibrosis Treatment by Nitric Oxide Inhalation

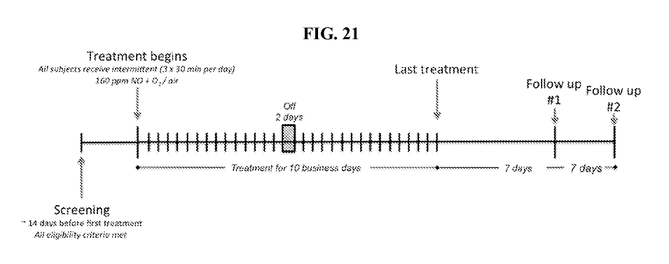

A cohort of human subjects diagnosed with cystic fibrosis were treated with intermittent inhalation of nitric oxide according to the methods described below and outlined in FIG. 21.

Subjects were screened within 14 days prior to the first study treatment. For inclusion in the study, subjects were required to be greater than or equal to 10 years old, diagnosed with CF, not O₂-dependent (i.e., resting awake O₂ saturation of at least 92% in room air), FEV1 of 30% to 85%, and to be colonized with P. aeruginosa and/or S. aureus.

Subjects diagnosed with methemoglobinemia, immune deficiency, or heart disease were excluded from the study. Subjects treated for high blood pressure, subjects on systemic steroids (1 mg/kg or greater than 20 mg of prednisone per day) within 30 days of screening, smokers, and subjects with a history of lung transplantation were also excluded. Other key exclusion criteria were as follows: MetHb greater than 3% at screening, pulmonary exacerbation resulting in antibiotic treatment (except prophylactic antibiotics) within 1 month before enrolment, history of frequent epistaxis (greater than 1 episode/month), and significant hemoptysis within 30 days (greater than or equal to 5 mL of blood in one coughing episode or greater than 30 mL of blood in a 24-hour period.

Following the 2-week treatment period, all subjects were required to return to the clinic for 2 follow-up assessments (e.g., physical exam, lung function tests, laboratory tests, sputum analysis, and AE/SAE questionnaires), 1 week and 2 weeks after the last study treatment.

Study Treatment:

The study treatment administered was as follows:
Investigational treatment: 800 ppm (0.08%) NO with 99.999% nitrogen purity balanced with N₂; delivered by inhalation mask at 160 ppm NO (with a blend of air and O₂ at a minimum concentration of 21% O₂).

In addition to their standard care, subjects were administered three 30-minute inhalations per day of 160 ppm NO (with O₂/air) for 10 working days (i.e., 5 days on treatment, 2 days off treatment, 5 days on treatment, for a total of 10 treatment days and 30 inhalations).

A minimum time interval of 3.5 hours from the end of one treatment to the beginning of the next treatment was required.

Oxygen and compressed air were delivered from the hospital user points to an O₂ microblender (Carefusion BIRD MODEL 03800) where it was blended to reach a pre-defined O₂ concentration that would allow a minimum concentration of 21% O₂ in the inhaled gas mixture. The mixed blend of O₂/air flow was controlled by an O₂ flow meter and delivered through the O₂ tubing.

An 800 ppm NO gas cylinder was utilized as a NO gas source. NO gas was regulated by a NO regulator. It was then delivered via a stainless steel high pressure hose to a NO mass flow meter, where the NO gas flow was regulated and delivered through the NO tubing into the breathing circuit.

Using a 3-way valve, NO and O₂ tubes were combined to deliver 160 ppm NO through NO tubing to the patient inhalation face mask.

NO (ppm), NO₂ (ppm) and O₂ (%) concentrations delivered to the patient were continuously monitored from a sampling port, using a dedicated monitor (AeroNox International Biomedical, US).

As a safety measure, during NO administration, human subjects' MetHb and 02 saturation levels in the blood were monitored utilizing a commercial “off the shelf” co-oxymeter (Masimo Corporation Model RAD-57/RAD 87).

In the event of MetHb greater than 5% or O₂ saturation less than or equal to 88%, treatment was temporarily discontinued, and measure repeated 30 minutes later. If value(s) had returned to above the safety threshold, the next inhalation was started according to protocol. In any case of a second episode of MeHb greater than 5%, study treatment was to be permanently discontinued.

Standard care for these subjects included oral antibiotics as well as tobramycin inhalation solution (TOBI), which is given on a monthly basis (one month with, one month without). Study treatment was given on the month that they were not given TOBI. Standard care also included continuous oral azithromycin; inhaled DNase (Pulmozyme); inhaled 3% saline and bronchodilators; inhaled antibiotics other than TOBI (e.g., colistin); daily vitamin supplementation (A,D,E,K); chest physiotherapy (postural drainage); supplemental high-protein, high-caloric food; and more.

Study Endpoints:

The primary study endpoints were safety and tolerability. There were no primary efficacy endpoints.

Primary Endpoints—Safety and Tolerability:
Safety
   Determine the MetHb levels during treatment with inhaled NO
   Determine other AEs associated with inhaled NO
Tolerability
   Proportion of subjects (%) who prematurely discontinued the study or study treatment for any reason
   Proportion of subjects (%) who prematurely discontinued the study treatment due to AEs or SAEs
   Proportion of subjects (%) who prematurely discontinued the study treatment due to AEs or SAEs associated with inhaled NO

Secondary Endpoints—Efficacy:
Comparison of FEV₁ improvement before and after NO treatment Observational Endpoints:
To determine the reduction in bacterial and fungal sputum load during each week of NO treatment compared to bacterial sputum load before NO treatment
Assess the improvement of lung function indices during and after NO intermittent inhalation treatment as determined by spirometry (i.e., FVC, FEV₁, FEV₁/FVC, forced expiratory flow 25% to 75% (FEF 25-75))
Evaluate C-reactive protein (CRP) levels over time
Number of subjects with study drug related bleeding at any time point

Analysis Sets:

The following analysis sets were defined for analysis of safety and efficacy:
ITT: Defined as all subjects who received at least one study treatment. This was the main set for analysis of efficacy and safety
PP: Defined as all subjects in the ITT cohort who completed study treatment in compliance with the protocol (i.e., excludes all early termination subjects) and who had no major protocol violations. This was a secondary set for analysis of efficacy

Statistical and Analysis Methods:

All measured variables and derived parameters were listed individually and tabulated by descriptive statistics. For categorical variables, summary tables were provided giving sample size, absolute and relative frequency. For continuous variables, summary tables were provided giving sample size, arithmetic mean, SD, median, minimum and maximum and 95% CI for means of variables. For the ITT cohort, the LOCF approach was applied, when deemed appropriate, to account for missing data at or prior to study termination. Methemoglobin was summarized in appropriate descriptive tables. The percent of subjects with MetHb level greater than 5% was calculated per time point. Changes from baseline in MetHb levels were analyzed using Signed rank test for two means. Laboratory results were summarized in appropriate tables by time. Changes from baseline were calculated and presented as well.

The Signed rank test for two means was applied for analyzing changes from baseline in continuous parameters:
FEV₁%
Specific bacterial colonization (i.e., S. aureus, P. aeruginosa)

All tests were two-tailed, and a p-value of 5% or less was considered statistically significant. The data was analyzed using the SAS® version 9.3 (SAS Institute, Cary N.C.). The planned sample size was 10 subjects. It should be noted that the results reported herein are considered preliminary.

Study Subjects:

The study population includes 9 subjects greater than or equal to 10 years old with CF and colonized with P. aeruginosa and/or S. aureus. The study was conducted at 2 centers: the Soroka University Medical Center, Pediatric Pulmonary Clinic and the Schneider Children's Medical Center, Cystic Fibrosis clinic. A total of 9 subjects were enrolled into the study, and all 9 subjects completed the entire study duration. All of the 9 subjects were included in the analyses of safety and efficacy.

Demographics and Baseline Characteristics:

A summary of the primary demographic characteristics at screening and baseline MetHb (pre-treatment values on Day 1) is provided in Table. Of the 9 subjects, 2 (22.2%) were male and 7 (77.8%) were female. All subjects were of Jewish ethnicity. The mean (SD) age was 28.89 (9.87) years and ranged from 13 to 46 years. The mean (SD) baseline MetHb was 1.03 (0.50)% and ranged from 0.30 to 1.70%. As part of the inclusion criteria, subjects were required to have a history of colonization with Pseudomonas aeruginosa and/or Staphylococcus aureus. A summary of the colonization history, by individual subject, is provided in Table. All 9 subjects had a history of P. aeruginosa colonization and 6/9 subjects also had a history of colonization with S. aureus.

TABLE 15
Summary of Demography and Baseline Characteristics (All Subjects, N = 9)

| Demographic Variable | All Subjects (N = 9) | |
|---|---|---|
| Gender (n (%)) | | |
| Male | 2 | (22.2%) |
| Female | 7 | (77.8%) |
| Ethnicity (n (%)) | | |
| Jewish | 9 | (100.0%) |
| Age (months) | | |
| n | 9 | |
| Mean (SD) | 28.89 | (9.87) |
| Median | 27.00 | |
| Min/max | 13/46 | |
| MetHb at baseline (%)(1) | | |
| n | 9 | |
| Mean (SD) | 1.03 | (0.50) |

| | |
|---|---|
| Median | 1.10 |
| Min/max | 0.30/1.70 |

1Pre-treatment value on Day 1.
Notes:
Max = Maximum; Min = Minimum; SD = Standard deviation.

TABLE 16
History of Colonization with Pseudomonas aeruginosa and/or
Staphylococcus aureus (All Subjects, N = 9)

| | Confirmed History of | Confirmed History of |
|---|---|---|
| | Colonization with | Colonization with |
| | Pseudomonas aeruginosa | Staphylococcus aureus |
| Subject | (Yes/No) | (Yes/No) |
| SC-01 | Yes | No |
| SC-02 | Yes | No |
| SC-03 | Yes | Yes |
| SC-04 | Yes | Yes |
| SC-05 | Yes | No |
| SO-01 | Yes | Yes |
| SO-02 | Yes | Yes |
| SO-03 | Yes | Yes |
| SO-04 | Yes | Yes |

Notes:
As part of the inclusion criteria, subjects were required to be colonized with Pseudomonas aeruginosa and/or Staphylococcus aureus

Medical History:

Medical history data was collected for the following body systems: allergic, cardiology, dermatological, ear nose and throat, hepatic, gastrointestinal, genitourinary, metabolic, ophthalmologic, renal, respiratory and other. A summary of subjects with medical history findings (abnormal clinically significant and abnormal not clinically significant) is provided in Table. The medical history data was consistent with this patient population. A history of abnormal clinically significant respiratory medical history was reported for all 9 subjects, and 5 (55.6%) subjects had abnormal gastrointestinal medical history (4 clinically significant and 1 not clinically significant).

TABLE 17
Summary of Medical History (All, N = 9)

| | All |
|---|---|
| | (N = 9) |
| Body System | n (%) |
| Any abnormal finding reported | 9 (100.0%) |
| Allergic | |
| Abnormal NCS | 3 (33.3%) |
| Abnormal CS | 2 (22.2%) |
| Ear nose and throat | |
| Abnormal NCS | 2 (22.2%) |
| Abnormal CS | 1 (11.1%) |
| Gastrointestinal | |
| Abnormal NCS | 1 (11.1%) |
| Abnormal CS | 4 (44.4%) |
| Genitourinary | |
| Abnormal NCS | 0 (0.0%) |
| Abnormal CS | 1 (11.1%) |
| Hepatic | |
| Abnormal NCS | 0 (0.0%) |
| Abnormal CS | 1 (11.1%) |
| Metabolic | |
| Abnormal NCS | 2 (22.2%) |
| Abnormal CS | 2 (22.2%) |
| Renal | |
| Abnormal NCS | 2 (22.2%) |
| Abnormal CS | 1 (11.1%) |
| Respiratory | |
| Abnormal NCS | 0 (0.0%) |
| Abnormal CS | 9 (100%) |

Notes:
CS = Clinically significant; NCS = Not clinically significant.

Exposure and Treatment Compliance:

Seven of the 9 subjects received all 30 treatments, and 2 subjects received 29/30 treatments. Of the treatments administered, the majority (265/268 (98.9%)) were complete 30 minute inhalations; 3 subjects had 1 treatment each that was less than 30 minutes in duration.

MetHb Percentage Associated with Inhaled NO:

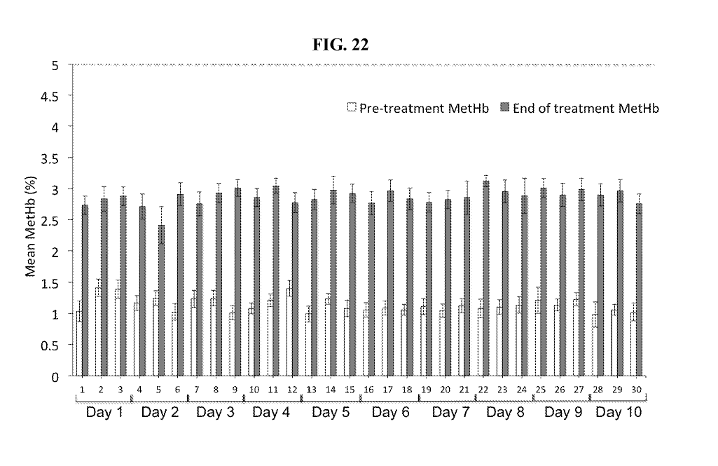

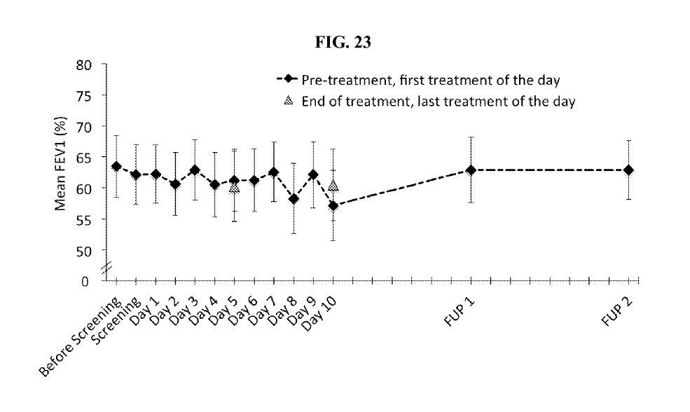

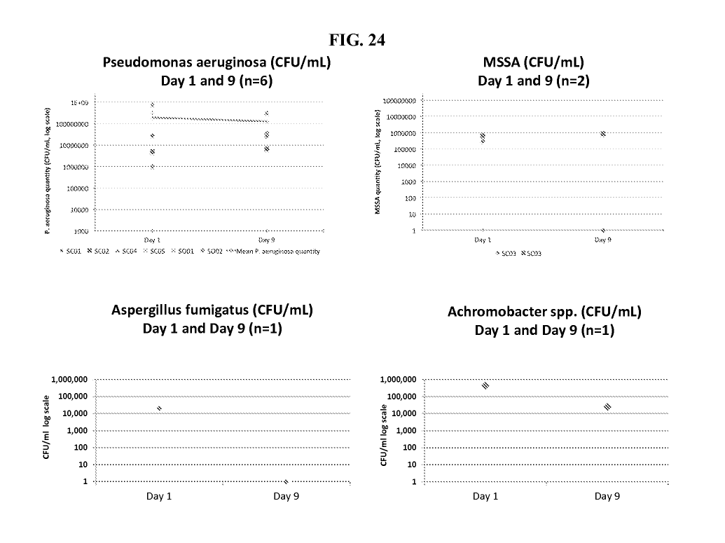

There were no subjects with MetHb greater than 5% during the entire study treatment period. The maximum MetHb level observed was 4.6%. As shown in FIG. 22, when comparing pre-treatment and end of treatment MeHb levels for each treatment in the study, there was no “cumulative” effect on MetHb levels over the study treatment period.

Efficacy Evaluation—FEV₁ Pre and Post-Treatment:

There was little change in mean FEV₁ before and after treatment and over time during the study. From Day 1 to Day 10, the mean (SD) FEV₁ change was −0.11% (5.5%) (p=0.992).

Observational Endpoints—Bacterial and Fungal Sputum Load:

Results of the bacterial and fungal sputum load analysis were highly variable, and due to limitations of the sampling and testing technique, must be interpreted with caution. Microbiology results were recorded from Day 1 to end of follow-up, for all samples taken during the study. In order for NO therapy to be efficient, a minimal exposure of the microbial population to NO for the whole duration of the study is required. NO first depletes the thiol protection mechanisms within the microbe before having a lethal effect; and in vitro studies suggest a minimum of 10 un-interrupted 30 minutes treatment is required. Thus, data was analyzed only for cultures with positive results on Day 1. Furthermore, samples from 2 subjects (SO-01 and SO-02) were not analyzed by the central laboratory. A summary of the bacterial and fungal sputum results at Day 1 and Day 9 is presented according to subject in Table 18.

TABLE 18
Summary of Bacterial and Fungal Sputum Results by Subject

| | | CFU/mL × 10⁻⁴ and % Change from Day 1 to Day 9 | | | | | | |
|---|---|---|---|---|---|---|---|---|
| | | | | Total | Total | | | |
| | | Non- | | P. aeruginosa | P. aeruginosa | | | |
| | | Mucoid | Mucoid | (central lab | (including | S. aureus | Aspergillus | Achromobacter |
| Subject | Visit | P. aeruginosa | P. aeruginosa | only) | Soroka lab) | (MSSA) | fumigatus | spp. |
| SC-01 | Day 1 | — | 2,700 | 2,700 | 2,700 | — | — | — |
| | Day 9 | — | 2,600 | 2,600 | 2,600 | — | — | — |
| | % change | — | −3.7% | −3.7% | −3.7% | | | |
| SC-02 | Day 1 | 496 | — | 496 | 496 | — | — | — |
| | Day 9 | 660 | — | 660 | 660 | — | — | — |
| | % change | +33.1% | | +33.1% | +33.1% | — | — | — |
| SC-03 | Day 1 | — | — | — | — | 29.5 | 2.00 | 44.1 |
| | Day 9 | — | — | — | — | 0 | 0 | 2.50 |
| | % change | — | — | — | — | −100.0% | −100.0% | −94.3% |
| SC-04 | Day 1 | 80,000 | — | 80,000 | 80,000 | — | — | — |
| | Day 9 | 33,700 | — | 33,700 | 33,700 | — | — | — |
| | % change | −57.9% | — | −57.9% | −57.9% | | — | |
| SC-05 | Day 1 | 29,200 | 29,000 | 32,100 | 32,100 | — | — | — |
| | Day 9 | 3,110 | 30.0 | 3,140 | 3,140 | — | — | — |
| | % change | −89.3% | −99.9% | −90.2% | −90.2% | | | |
| SO01 | Day 1 | — | — | — | 450 | — | — | — |
| | Day 9 | — | — | — | 3,200 | — | — | — |
| | % change | — | — | — | +611% | — | — | — |
| SO02 | Day 1 | — | — | — | 100 | — | — | — |
| | Day 9 | — | — | — | 350 | — | — | — |
| | % change | — | — | — | +250% | — | — | — |
| SO03 | Day 1 | — | — | — | — | 67.0 | — | — |
| | Day 9 | — | — | — | — | 85.5 | — | — |
| | % change | — | — | — | — | +27.6% | — | — |
| SO04 | Day 1 | — | — | — | — | — | — | — |
| | Day 9 | — | — | — | — | — | — | — |

Notes:
Data was collected only for bacterial counts that were higher than 0 on Day 1.
CFU = Colony forming unit;
MSSA = Methicillin-susceptible Staphylococcus aureus.
“—” indicates not applicable.

Lung Function Indices Over Time:

Lung function indices remained relatively constant throughout the study, and there were no clinically significant changes reported. A summary of the mean FVC absolute, FVC predicted (%), FEV₁ absolute, FEV₁ predicted (%), FEV₁/FVC, FEF 25-75 absolute, and FEF 25-75 predicted (%) is provided in Table.

TABLE 19
Summary of Lung Function Indices Over Time (All Subjects)

| | FVC | FVC | FEV1 | FEV1 | FEV1/ | FEF 25-75 | FEF 25-75% |
|---|---|---|---|---|---|---|---|
| | Absolute | Predicted | Absolute | Predicted | FVC | Absolute | Absolute |
| Variable | (Liter) | (%) | (Liter) | (%) | (%) | (Liter/sec) | (%) |
| Day 1 | | | | | | | |
| n | 9 | 9 | 9 | 9 | 9 | 9 | 9 |
| Mean | 2.756 | 79.889 | 1.871 | 62.222 | 68.778 | 1.151 | 30.000 |
| (SD) | (0.970) | (15.054) | (0.706) | (14.078) | (4.816) | (0.511) | (13.901) |
| Median | 2.360 | 90.000 | 1.700 | 64.000 | 69.000 | 1.090 | 27.000 |
| Min/max | 1.720/4.760 | 59.000/95.000 | 1.080/3.280 | 38.000/77.000 | 60.000/78.000 | 0.410/1.930 | 10.000/55.000 |
| Day 10 | | | | | | | |
| n | 9 | 9 | 9 | 9 | 9 | 9 | 9 |
| Mean | 2.759 | 79.556 | 1.833 | 60.444 | 65.111 | 1.090 | 28.111 |
| (SD) | (1.026) | (16.486) | (0.817) | (17.256) | (7.623) | (0.696) | (16.714) |
| Median | 2.340 | 84.000 | 1.610 | 59.000 | 66.000 | 0.880 | 22.000 |
| Min/max | 1.880/4.900 | 59.000/102.000 | 1.080/3.540 | 39.000/83.000 | 55.000/78.000 | 0.390/2.470 | 10.000/54.000 |
| Follow-up 2 | | | | | | | |
| n | 9 | 9 | 9 | 9 | 9 | 9 | 9 |
| Mean | 2.786 | 81.889 | 1.852 | 62.889 | 66.000 | 1.118 | 29.667 |
| (SD) | (0.902) | (14.538) | (0.674) | (14.304) | (7.984) | (0.599) | (18.317) |
| Median | 2.390 | 85.000 | 1.630 | 60.000 | 65.000 | 0.910 | 23.000 |
| Min/max | 1.920/4.600 | 64.000/108.000 | 1.170/3.200 | 41.000/79.000 | 55.000/83.000 | 0.400/1.980 | 10.000/70.000 |

Notes:
Follow-up-2 was 2 weeks after Day 10 of treatment.
Max = Maximum;
Min = Minimum;
SD = Standard deviation.

CRP Levels Over Time:

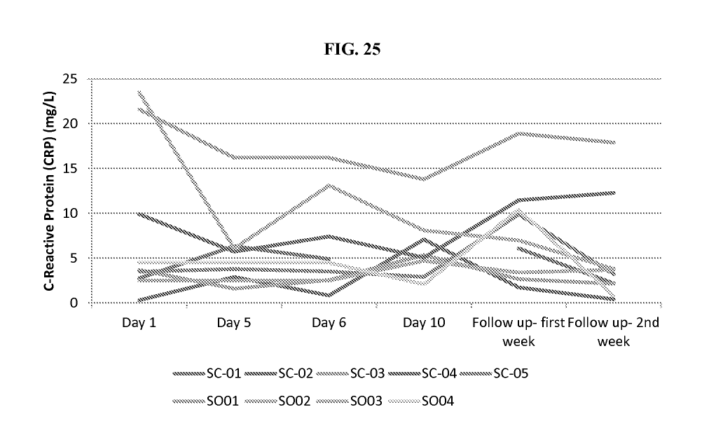

For most subjects, CRP was less than or equal to 5 mg/mL which is below the threshold considered indicative of systemic inflammation. For these subjects, although CRP levels fluctuated over time, levels remained mainly below 5 mg/mL. Three subjects had systemic inflammation (CRP greater than 5 mg/L) on Day 1, and for these subjects, CRP levels decreased from Day 1 to Day 10 (i.e., during the treatment period) (see FIG. 25).

Summary and Conclusions: In this study of 9 CF subjects (greater than or equal to 10 years old), inhalations of 160 ppm NO for 30 minutes, 3 times daily, for 10 treatments over a 2-week treatment period (in addition to standard O₂ treatment) were well tolerated.

Adverse events were reported by 5 (55.5%) subjects. There were no severe or serious AEs, no treatment withdrawals due to AEs, and no deaths. Adverse events considered by the Investigator as possibly or probably related to treatment were reported for 2 (22.2%) subjects. Of the 31 total AEs reported, the majority of events (20) occurred in a single subject (Subject SC-01).

There was no AEs of MetHb elevation greater than 5% or NO₂ elevation greater than 5 ppm. In total, 7 cases of haemoptysis were reported in 2 subjects and all events were mild in severity. There were no subjects with MetHb greater than 5% at any point during the study and there was no no cumulative effect of MetHb exposure during the study. The maximum MetHb level reported was 4.6%.

No primary efficacy analyses were conducted in this small (N=9) uncontrolled, short-term study in human subjects with clinically stable CF. There were no statistically significant or clinically relevant changes in FEV₁ over time, and lung function indices also remained relatively constant throughout the study duration. Although results of the bacterial and fungal sputum load analysis were highly variable, marked reductions of MSSA, Achromabacter, P. aeruginosa, and Aspergillus were seen in some subjects. In subjects with systemic inflammation (CRP greater than 5 mg/mL) at baseline, CRP levels decreased over the treatment period.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method for treating a disease in a human subject in need thereof,
wherein the disease is selected from the group consisting of inflammation, bronchiolitis and cystic fibrosis, and
wherein the method comprises repeatedly administering to the human subject a gas mixture comprising nitric oxide at a concentration from about 144 to about 176 ppm for a first period of time, followed by a gas mixture containing no nitric oxide for a second period of time, wherein the administration is repeated for a time sufficient to:
a) reduce the level of at least one inflammatory biomarker in the human subject when compared to the level of the inflammatory biomarker prior to the administration;
b) reduce the microbial density by 1 to 2 log units as measured by colony forming units in the human subject when compared to the microbial density prior to the administration; or
c) a combination thereof.

2. The method of claim 1, wherein the human subject suffers from a microbial infection associated with cystic fibrosis.

3. The method of claim 2, wherein the microbial infection is caused by a pathogenic microorganism.

4. The method of claim 3, wherein said pathogenic microorganism is selected from the group consisting of P. alcaligenes, non-mucoid and mucoid Pseudomonas aeruginosa, A. fumigates, Staphylococcus aureus, Haemophilus influenza, Burkholderia cepacia complex, Klebsiella pneumonia, Escherichia coli, methicillin-resistant Staphylococcus aureus (MRSA), methicillin-sensitive Staphylococcus aureus (MSSA), Stenotrophomonas maltophilia, Achromobacter spp., Achromobacter xylosoxidans and non-tuberculous mycobacteria (NTM) species.

5. The method of claim 1, wherein the first time period is 30 minutes and the second time period is from about 3 to about 5 hours.

6. The method of claim 1, wherein the administration is repeated 6 times per day.

7. The method of claim 1, wherein the nitric oxide is repeatedly administered for a period of time from about one day to three weeks.

8. The method of claim 1, wherein the nitric oxide is repeatedly administered for 5 days.

9. The method of claim 1, wherein the at least one inflammatory biomarker is selected from the group consisting of C-reactive protein (CRP), TNFα, TNF RII, IL-1β, IL-1ra/IL-1F3, IL-2, IL-4, IL-5, IL-6, IL-8, CXCL8/IL-8, IL-10, IL-12 p70, IL-17A, GM-CSF, ICAM-1, IFN-gamma, MMP-8, MMP-9, VEGF and IL-12p70, neutrophils, lymphocytes and eosinophils count, neutrophil elastase activity, alpha-1-antitrypsin (AAT), haptoglobin, transferrin, an immunoglobulin, granzyme B (GzmB), eosinophil cationic protein (ECP), eotaxin, tryptase, chemokine C-C motif ligand 18 (CCL18/PARC), RANTES (CCL5), surfactant protein D (SP-D), lipopolysaccharide (LPS)-binding protein and soluble cluster of differentiation 14 (sCD14).

10. The method of claim 1, wherein the at least one inflammatory biomarker is C-reactive protein (CRP).

11. The method of claim 1, further comprising monitoring at least one on-site oximetric parameter in the subject, said on-site parameter being selected from the group consisting of: oxyhemoglobin saturation (SpO₂); methemoglobin (SpMet); perfusion index (PI); respiration rate (RRa); oxyhemoglobin saturation (SpO₂); total hemoglobin (SpHb); carboxyhemoglobin (SpCO); methemoglobin (SpMet); oxygen content (SpOC); and pleth variability index (PVI).

12. The method of claim 1, further comprising monitoring at least one additional on-site spirometric parameter in the subject, said at least one additional on-site parameter being selected from the group consisting of: forced expiratory volume (FEV1); maximum mid-expiratory flow (MMEF); diffusing capacity of the lung for carbon monoxide (DLCO); forced vital capacity (FVC); total lung capacity (TLC); and residual volume (RV).

13. The method of claim 1, further comprising monitoring at least one on-site parameter in said gas mixture inhaled by the subject, said on-site parameter being selected from the group consisting of: end tidal CO₂ (ETCO₂); nitrogen dioxide (NO₂), nitric oxide (NO); serum nitrite/nitrate; and fraction of inspired oxygen (FiO₂).

14. The method of claim 1, further comprising monitoring at least one off-site bodily fluid parameter in the subject, said parameter being selected from the group consisting of: a bacterial and/or fungal load; urine nitrite; blood methemoglobin; blood pH; a coagulation factor; blood hemoglobin; hematocrit ratio; red blood cell count; white blood cell count; platelet count; vascular endothelial activation factor; renal function; an electrolyte; a pregnancy hormone; serum creatinine; and liver function.
